(19) United States
(12) Patent Application Publication
(10) Pub. No.: US 2025/0101419 A1
(43) Pub. Date: Mar. 27, 2025

(54) ALLELE SPECIFIC SPLICE SWITCHING OLIGONUCLEOTIDES TARGETING PSEUDOEXONS

(71) Applicant: SYDDANSK UNIVERSITET, Odense M (DK)

(72) Inventors: Brage Storstein ANDRESEN, Åbyhøj (DK); Thomas Koed DOKTOR, Broby (DK); Lise Lolle HOLM, Næstved (DK); Ulrika Simone Spangsberg PETERSEN, Odense C (DK); Gitte Hoffmann BRUUN, Odense S (DK)

(21) Appl. No.: 18/728,263

(22) PCT Filed: Jan. 26, 2023
(86) PCT No.: PCT/EP2023/051916

§ 371 Date: Jul. 11, 2024

Related U.S. Application Data
(63) Continuation of application No. PCT/EP2022/051790, filed on Jan. 26, 2022.

(30) Foreign Application Priority Data
Jun. 29, 2022 (DK) PA202270346

(51) Int. Cl. C12N 15/113

(52) U.S. Cl. CPC C12N 15/113; C12N 2310/11; C12N 2320/33

(57) ABSTRACT

The present invention relates to a method for identifying splice switching oligonucleotides (SSOs) able to modulate expression of a target protein in a cell by promoting incorporation of a pseudoexon into the mature mRNA upon binding to the pre-mRNA in the region +9 to +39 downstream to the 5′ splice site of said pseudoexon. The invention also relates to SSOs obtained by said method and uses thereof. In particular, the present invention relates to allele specific splice switching oligonucleotides (SSOs).

### TECHNICAL FIELD OF THE INVENTION

The present invention relates to allele specific splice switching oligonucleotides (SSOs) that can activate splicing of pseudoexons. In particular, the SSOs are able to promote inclusion of the pseudoexon in an mRNA transcript of a gene, such as in an allele specific manner, thereby inhibiting expression of a functional gene product. In another aspect, the invention relates to a method for identifying pseudoexons for which it is possible to incorporate the pseudoexons in mature mRNAs using the SSOs.

### BACKGROUND OF THE INVENTION

Newly synthesized eukaryotic mRNA molecules, also known as primary transcripts or pre-mRNA, made in the nucleus, are processed before or during transport to the cytoplasm for translation. Processing of the pre-mRNAs includes addition of a 5′ methylated cap and an approximately 200-250 nucleotides poly(A) tail to the 3′ end of the transcript.

Another step in mRNA processing is splicing of the pre-mRNA, which is part of the maturation of 90-95% of mammalian mRNAs. Introns (or intervening sequences) are regions of a primary transcript that are not included in the coding sequence of the mature mRNA. Exons are regions of a primary transcript that remain in the mature mRNA when it reaches the cytoplasm. The exons are spliced together to form the mature mRNA sequence. Splicing occurs between splice sites that together form a splice junction. The splice site at the 5′ end of the intron is often called the “5′ splice site,” or “splice donor site” and the splice site at the 3′ end of the intron is called the “3′ splice site” or “splice acceptor site”. In splicing, the 3′ end of an upstream exon is joined to the 5′ end of the downstream exon. Thus, the unspliced RNA (or pre-mRNA) has an exon/intron splice site at the 5′ end of an intron and an intron/exon splice site at the 3′ end of an intron. After the intron is removed, the exons are contiguous at what is sometimes referred to as the exon/exon junction or boundary in the mature mRNA. Alternative splicing, defined as the splicing together of different combinations of exons or exon segments, often results in multiple mature mRNA transcripts expressed from a single gene.

The splicing of precursor mRNA (pre-mRNA) is an essential step in eukaryotic gene expression, where introns are removed through the activities of the spliceosome, and the coding parts of a gene are spliced together, resulting in a functional mRNA. Pre-mRNA splicing is a highly controlled process, and it is well established that mutations can impact splicing and generate aberrant transcripts. Correct mRNA splicing depends on regulatory sequences, which are recognized by different factors of the spliceosome, as well as splicing regulatory factors. The splicing regulatory factors either stimulate or repress recognition and splicing of exons by sequence specific binding to splicing regulatory sequences such as splicing enhancers and splicing silencers. Pre-mRNA splicing in eukaryotes is often associated with extensive alternative splicing to enrich their proteome. Alternative selection of splice sites permits eukaryotes to modulate cell type specific gene expression, contributing to their functional diversification. Alternative splicing is a highly regulated process influenced by the splicing regulatory proteins, such as SR proteins or hnRNPs, which recognize splicing regulatory sequences, such as exonic splicing enhancers (ESEs) and exonic splicing silencers (ESSs) in exons, and intronic splicing enhancers (ISEs) and intronic splicing silencers (ISSs) in introns.

It is a well-known fact that exonic mutations, which either create or eliminate existing splicing regulatory sequences other than the splice site sequences often lead to mis-splicing of the RNA that might result in diseases. However, it is difficult to predict which mutations affect splicing as not all exons are critically dependent on splicing regulatory elements other than the splice sites, and consequently only a limited number of exons are vulnerable to mutations in splicing regulatory sequences outside of the splice sites.

### SUMMARY OF THE INVENTION

The present invention relates to the identification of sequence parameters in a gene comprising a pseudoexon, which can be used to determine if it is possible to get the pseudoexon incorporated into the mature mRNA using a splice switching oligonucleotide (SSO). Incorporation of pseudoexons can e.g. be used to inactivate, disrupt, or alter the function of the functional product expressed from a gene by incorporation of the pseudoexon in the mature mRNA (see also example 1 and corresponding figure and figure legend for further information). The invention also relates to (medical) uses of such SSOs.

In one method of the invention, the inventing team has identified numerous SNPs within SSO binding regions allowing for allele specific targeting. Allele-specific SSOs can be used for preferentially targeting the disease-causing pre-mRNA (from the disease causing allele), whereas the pre-mRNA from the “normally functioning” allele is unaffected (or less affected). (see example 13).

In another method of the invention, the inventing team has identified SNPs within the splice site sequences of pseudoexons allowing for allele specific incorporation of the pseudoexon in the mature RNA in only the disease-causing gene (and not the other “normal” gene on the other allele). Phrased in another way, when the MaxEnt score of a splice site in the disease causing gene, is increased compared to the other allele, allele specific targeting is possible (see examples 15-16).

Example 2 shows that the identified parameters are essential for identifying activatable pseudoexons (Table 1) and non-activatable pseudoexons (Table 2).

Example 3 shows data in relation to SMAD2 (see also example 11).

Example 12 shows data in relation to RNF115.

Examples 4-11 and 13-14 show further examples for specific genes comprising pseudoexons where the pseudoexons can be activated (incorporated in the mature mRNA).

Example 13, 15 and 16 show allele specific targeting.

Thus, an object of the present invention relates to the provision of sequence parameters (criteria) which can identify binding sites for SSOs for incorporation of pseudoexons into mature mRNA.

Another object of the invention is to provide SSOs, which, in vivo, can promote incorporation of pseudoexons into mature mRNA, thereby inactivating, disrupting, or altering the natural function of genes.

Thus, one aspect of the invention relates to a method for identifying SSOs able to modulate expression and/or function of a target protein in a cell by promoting incorporation of a pseudoexon into the mature mRNA upon binding to the pre-mRNA in the region +9 to +39 downstream to the 5′ splice site of said pseudoexon, the method comprising;
a) providing one or more gene sequences comprising one or more identified pseudoexons, such as in the form of a database or other storage means;
b) determining for the one or more gene sequences; if the pseudoexon meets the following criteria:
   Pseudoexon length<160 nt;
   Pseudoexon length>30 nt;
   The last 3 nt of the pseudoexon are different from TAG;
   Donor splice site has a MaxEnt score≥4.33;
   Donor splice site has a MaxEnt score≤10.06; and
   Acceptor splice site has a MaxEnt score≥3.63;
c) determining for the one or more gene sequences if the sequence region +9 to +39 downstream to the 5′ splice site of said pseudoexon (3) meets the following criteria:
   Total pyrimidines≤20;
   Total thymidine bases≤12;
   Total thymidine bases≥4;
   Total guanine bases≤12;
   Maximum length of thymidine polymer≤4;
   Maximum length of pyrimidine polymer≤10;
   Minimum length of purine polymer≥3; and
   Maximum number of guanine polymers of at least 3 nt length≤2;
      wherein, if one or more gene sequences meet the criteria according to point b) and point c), said region +9 to +39 downstream to the 5′ splice site of said pseudoexon (3) is considered a target for an SSO able to, in vivo, hybridize to the pre-mRNA (2) of said gene within the region +9 to +39 downstream to the 5′ splice site of said pseudoexon (3); and resulting in said pseudoexon (3) becoming part of the mature mRNA to a greater extent compared to corresponding pre-mRNA not contacted with the SSO (1);
      and
      optionally, producing said SSO, optionally for use as a medicament.

The present invention also relates to specifically identified SSO for use as medicaments. Thus, another aspect of the invention relates to a composition comprising a splice switching oligonucleotide (SSO) for use as a medicament, said composition comprising
an SSO complementary or substantially complementary to region within a nucleic acid selected from the group consisting of
   a nucleic acid according to any of SEQ ID NO's: 106, 1-26, 79-105, 107-125 and 137-201; or
   a nucleic acid comprising 1 or 2 or 3 substitutions when compared to any of any of SEQ ID NO's: 106, 1-26, 79-105, 107-125 and 137-201; or
   a nucleic acid sequence having at least 90% sequence identity to any of any of SEQ ID NO's: 106, 1-26, 79-105, 107-125 and 137-201;
      or
an SSO selected from the group consisting of:
   a nucleic acid according to any of SEQ ID NO's: 127-136 and SEQ ID NO's: 202-216; or
   a nucleic acid comprising 1 or 2 or 3 substitutions when compared to any of SEQ ID NO's: 127-136 and SEQ ID NO's: 202-216; or
   a nucleic acid sequence having at least 90% sequence identity to any of SEQ ID NO's: 127-136 and SEQ ID NO's: 202-216;
      wherein said SSO being complementary or substantially complementary to a target pre-mRNA (encoding a functional disorder-causing or disorder-influencing protein), said target pre-mRNA comprising:
a function-disabling pseudoexon comprising:
   at the 5′-end a 3′ splice site; and
   at the 3′-end a 5′ splice site;
      wherein said SSO is complementary or substantially complementary to the target pre-RNA at a region +9 to +39 downstream to the 5′ splice site of said pseudoexon;
      wherein, when said SSO, in vivo, hybridizes to the pre-mRNA within the region +9 to +39 downstream to the 5′ splice site of said pseudoexon; said pseudoexon becomes part of the mature mRNA to a greater extent compared to corresponding pre-mRNA not contacted with the SSO.

In yet an aspect the invention relates to a
a composition comprising an SSO complementary or substantially complementary to region within a nucleic acid selected from the group consisting of
   a nucleic acid according to any of any of SEQ ID NO's: 106, 1-26, 79-105, 107-125 and 137-201; or
   a nucleic acid comprising 1 or 2 or 3 substitutions when compared to any of any of SEQ ID NO's: 106, 1-26, 79-105, 107-125 and 137-201; or
   a nucleic acid sequence having at least 90% sequence identity to any of any of SEQ ID NO's: 106, 1-26, 79-105, 107-125 and 137-201;
      or
a composition comprising an SSO selected from the group consisting of:
a nucleic acid according to any of SEQ ID NO's: 127-136 and SEQ ID NO's: 202-216; or
a nucleic acid comprising 1 or 2 or 3 substitutions when compared to any of SEQ ID NO's: 127-136 and SEQ ID NO's: 202-216; or
a nucleic acid sequence having at least 90% sequence identity to any of SEQ ID NO's: 127-136 and SEQ ID NO's: 202-216.

A further aspect of the invention relates to a composition comprising a splice switching oligonucleotide (SSO) for use as a medicament, said composition comprising
an SSO complementary or substantially complementary to a region within a nucleic acid selected from the group consisting of
   a nucleic acid according to any of SEQ ID NO's: 217-294;
   a nucleic acid comprising 1 or 2 or 3 substitutions when compared to any of SEQ ID NO's: 217-294; or
   a nucleic acid sequence having at least 90% sequence identity to any of SEQ ID NO's: 217-294;
      or
an SSO selected from the group consisting of:
   a nucleic acid according to any of SEQ ID NO's: 295-367; or
   a nucleic acid comprising 1 or 2 or 3 substitutions when compared to any of SEQ ID NO's: 295-367; or
   a nucleic acid sequence having at least 90% sequence identity to any of SEQ ID NO's: 295-367;
      wherein said SSO being complementary or substantially complementary to a target pre-mRNA, said target pre-mRNA comprising:
a function-disabling pseudoexon comprising:
   at the 5′-end a 3′ splice site; and
   at the 3′-end a 5′ splice site;
      wherein said SSO is complementary or substantially complementary to the target pre-mRNA at a region +9 to +39 downstream to the 5′ splice site of said pseudoexon.

The present invention may also be used for identifying subjects who are eligible for allele-specific SSO-based pseudoexon inclusion treatment. Thus, an aspect of the invention relates to a method for identifying a subject who is likely eligible for allele-specific SSO-based pseudoexon inclusion treatment of a dysfunctional or disease-causing gene, the method comprising
determining in a biological sample from said subject the presence or absence of heterozygosity in a pseudoexon of the dysfunctional or disease-causing gene;
   wherein if the subject is heterozygous for a sequence variation (SNP) in the 5′ splice site and/or the 3′ splice site, preferably the 5′ splice site of the function-disabling pseudoexon, said subject is likely eligible for allele-specific SSO-based pseudoexon inclusion treatment; or
   wherein if the subject is not heterozygous for a sequence variation (SNP) in the 5′ splice site and/or the 3′ splice site of the function-disabling pseudoexon, said subject is likely not eligible for allele-specific SSO-based pseudoexon inclusion treatment;
   wherein said SSO is complementary or substantially complementary to a target pre-mRNA of the dysfunctional or disease-causing gene, said target pre-mRNA comprising:
a function-disabling pseudoexon comprising:
   at the 5′-end a 3′ splice site; and
   at the 3′-end a 5′ splice site.

Yet an aspect of the invention relates to a method for identifying a subject who is likely eligible for SSO-based pseudoexon inclusion treatment of a dysfunctional or disease-causing gene, the method comprising
determining in a biological sample from said subject the presence or absence of a SNP, which enables SSO-based pseudoexon inclusion treatment, in a pseudoexon of the dysfunctional or disease-causing gene;
   wherein if the subject has a sequence variation (SNP) in the 5′ splice site and/or the 3′ splice site, preferably the 5′ splice site of the function-disabling pseudoexon, said subject is likely eligible for SSO-based pseudoexon inclusion treatment; or
   wherein if the subject does not have a sequence variation (SNP) in the 5′ splice site and/or the 3′ splice site of the function-disabling pseudoexon, said subject is likely not eligible for SSO-based pseudoexon inclusion treatment;
wherein said SSO is complementary or substantially complementary to a target pre-mRNA of the dysfunctional or disease-causing gene, said target pre-mRNA comprising:
a function-disabling pseudoexon comprising:
at the 5′-end a 3′ splice site; and
at the 3′-end a 5′ splice site.

### BRIEF DESCRIPTION OF THE FIGURES

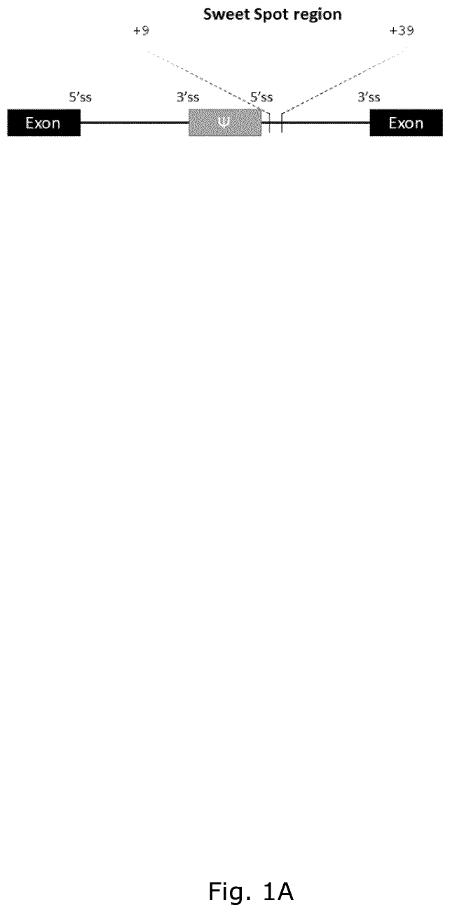

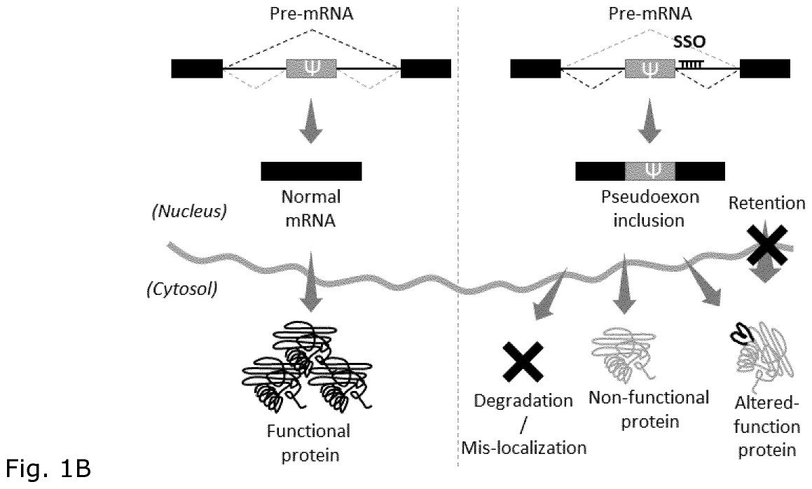

### FIG. 1

FIG. 1 shows a schematic overview of gene expression regulation by SSO according to the invention. FIG. 1A) a Sweet Spot region is identified in the intron part of the pre-mRNA as a region +9 to +39 nucleotides downstream for the 5′splice site of a pseudoexon that obeys the criteria according to the invention. FIG. 1B) Pseudoexon inclusion into the mRNA transcript can be activated and increased by employing SSOs complementary to the Sweet Spot region of pseudoexons fulfilling the criteria. Pseudoexon inclusion into the mRNA will modulate gene expression either at the mRNA level or protein level, by mislocalization, destabilization, degradation or alteration of mRNA or protein function.

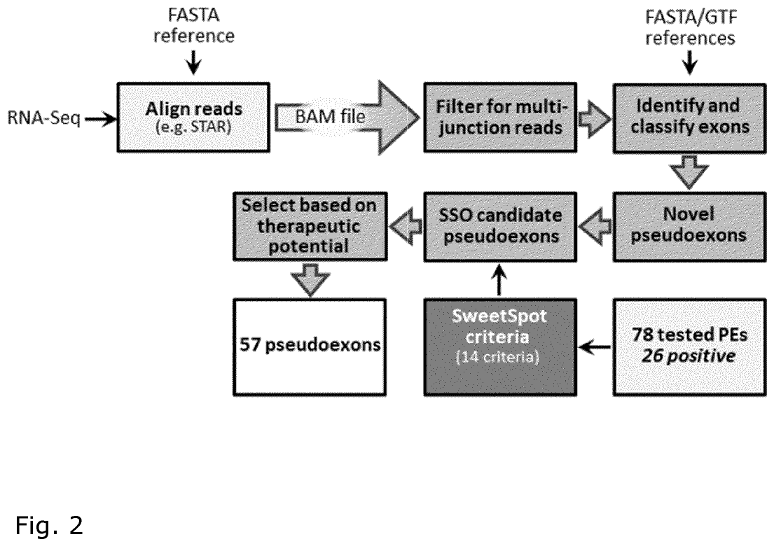

### FIG. 2

FIG. 2 shows a schematic demonstration of how RNA-sequencing data can be used in detection of in vivo spliced double junctions for empirical detection of pseudoexons, which are included into the endogenous transcript at low levels.

After mapping to the human genome, reads are filtered to retain only fragments containing at least two splicing junctions. The splicing junctions of the entire fragment are then assembled into an exon structure, allowing for an unmapped gap between reads in the fragment of up to 100 bp. Exons are then classified using known exon annotations to identify pseudoexons contained within introns. Novel pseudoexons that may be candidates for activation by SSOs binding to the Sweet Spot region can be identified by 14 criteria, after which highly therapeutically relevant pseudoexons can be identified in genes where a downregulation of expression or alteration of the functional gene product is medically relevant.

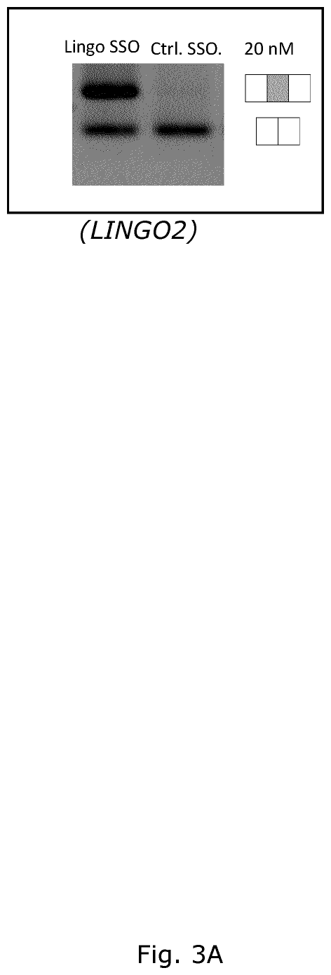

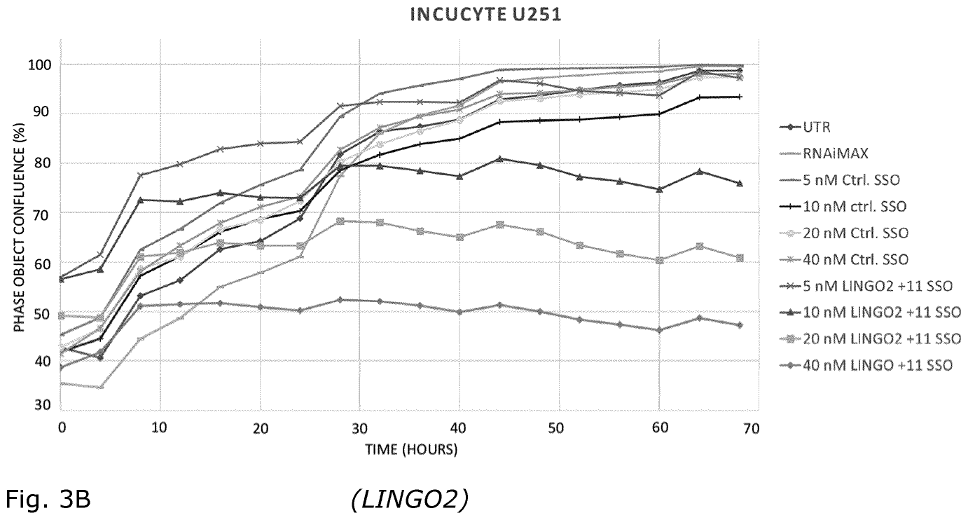

### FIG. 3

LINGO2 pseudoexon inclusion inhibits growth and proliferation of glioblastoma cells. (A) RT-PCR analysis of LINGO2 pseudoexon splicing in U251 cells transfected with the LINGO2 pseudoexon +11 SSO and a nontargeting SSO control. The upper band includes the pseudoexon, which is activated by transfection of the +11 SSO. (B) IncuCyte@cell proliferation assay showing growth curves of U251 cells transfected with the LINGO2+11 SSO and a nontargeting SSO control at different concentrations (cell confluency relative to time after transfection). The growth is inhibited by transfection of the +11 SSO in a dose-dependent manner.

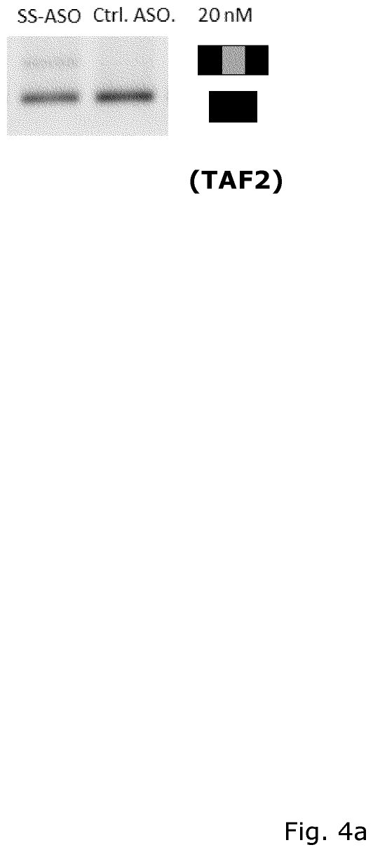

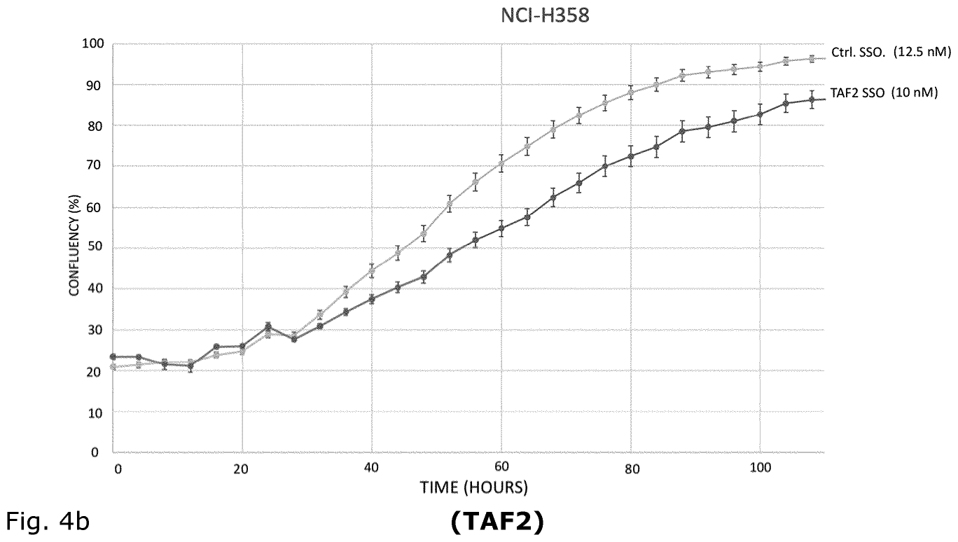

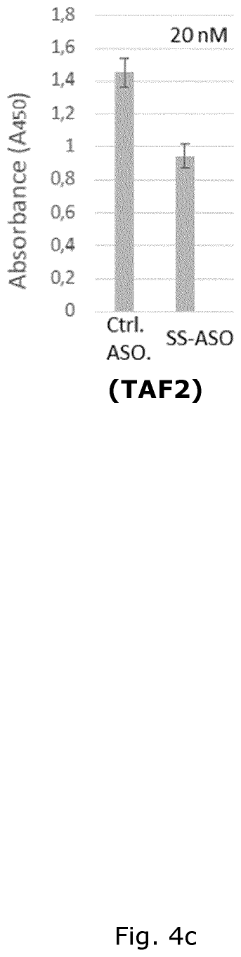

### FIG. 4

TAF2 pseudoexon inclusion inhibits growth and proliferation of lung cancer cells. (A) RT-PCR analysis of TAF2 pseudoexon splicing in NCI-H358 cells transfected with the TAF2 pseudoexon +11 SSO and a nontargeting SSO control. The upper band includes the pseudoexon, which is activated by transfection of the +11 SSO. (B) IncuCyte® cell proliferation assay showing growth curves of NCI-H358 cells transfected with the TAF2 pseudoexon+11 SSO and a nontargeting SSO control (cell confluency relative to time after transfection). The growth is inhibited by transfection of the +11 SSO. (C) Bar plots from WST-1 cell viability and growth assay showing absorbance at 450 nm as a measure of cell viability of NCI-H358 lung cancer cells transfected with the TAF2 pseudoexon +11 SSO and a nontargeting SSO control. The cell viability is decreased by transfection of the +11 SSO.

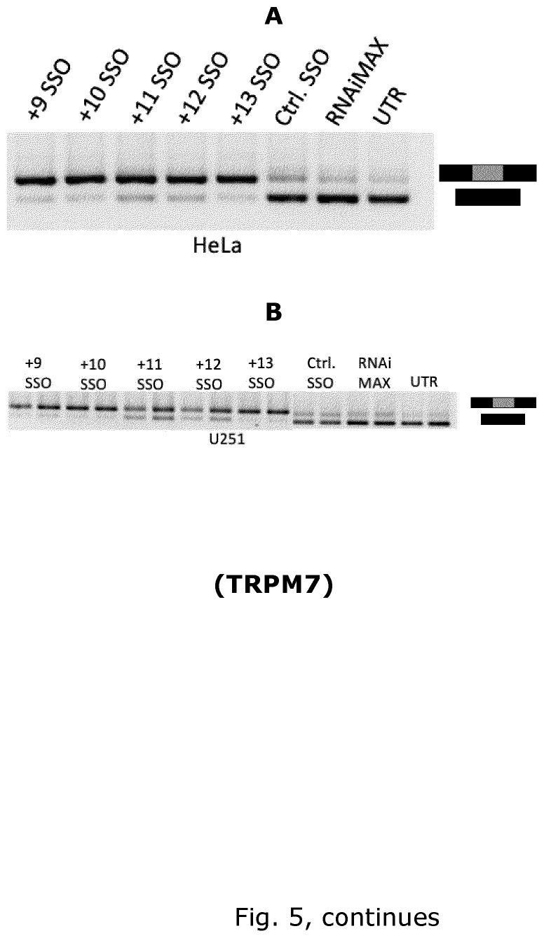

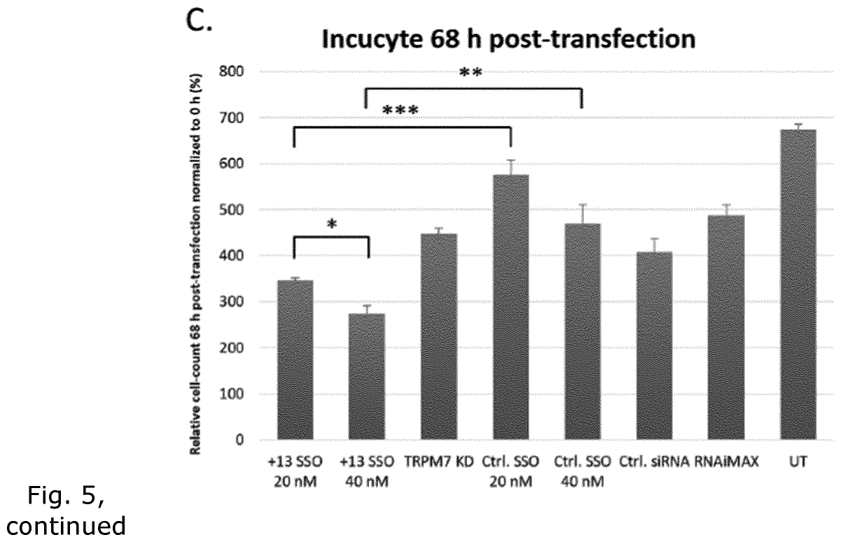

### FIG. 5

Optimization of TRPM7 SSOs targeting the Sweet Spot region. (A+B) RT-PCR analysis of TRPM7 pseudoexon splicing in (A) HeLa and (B) U251 cells transfected with 20 nM of the TRPM7+9, +10, +11, +12 and +13 SSOs, including controls; transfection of a nontargeting control SSO and untransfected cells with transfection reagent (RNAiMAX) or without (UTR). The upper band includes the pseudoexon and the amount is increased by transfection of the +9 to +13 SSOs. (C) Bar plots from IncuCyte® cell proliferation assay showing the relative cell count 68 hours after transfection of the TRPM7 pseudoexon +13 SSO and TRPM7 siRNA (KD), including controls; transfection of a nontargeting control SSO and untransfected cells with transfection reagent (RNAiMAX) or without (UTR). The growth is inhibited by transfection of the +13 SSO. Student's t test, *p<0.05, **p<0.01 and ***p<0.001.

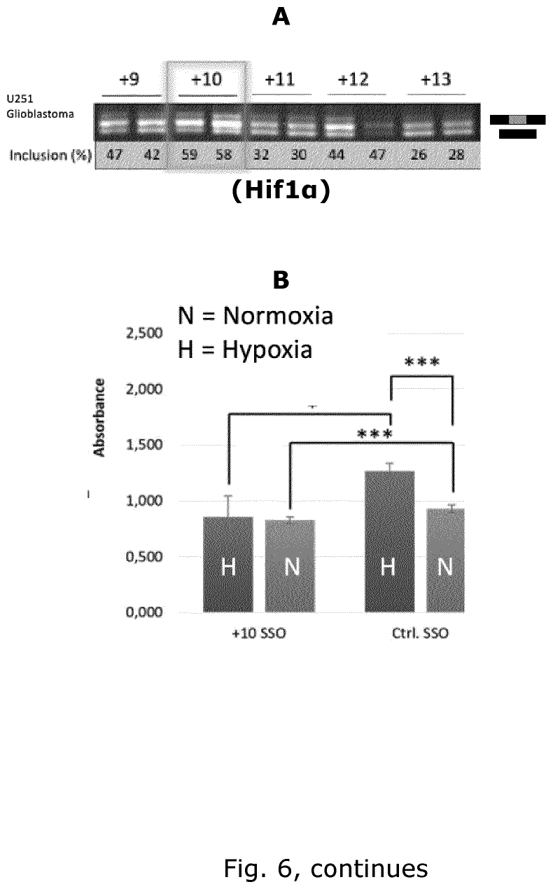

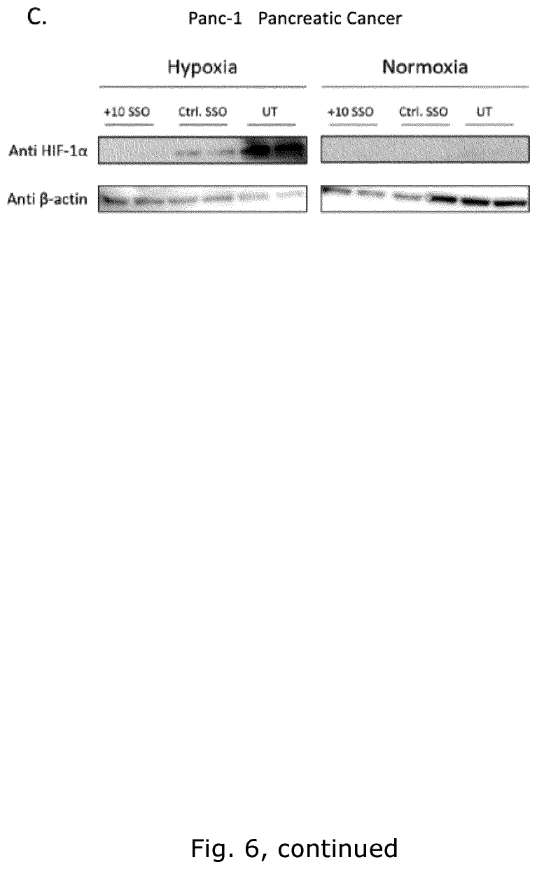

### FIG. 6

Optimization of HIF1A SSOs targeting the Sweet Spot region. (A) RT-PCR analysis of HIF1A pseudoexon splicing in U251 cells transfected with the HIF1A pseudoexon +9, +10, +11, +12 and +13 SSOs. The middle band includes the pseudoexon and the efficiency is highest by transfection with the +10 SSO. Pseudoexon inclusion levels were quantified using the Fragment Analyzer (Advanced Analytical Technologies). (B) WST-1 assay showing absorbance as a measure of growth and proliferation of U251 cells transfected with 20 nM of the HIF1A pseudoexon +10 SSO and a nontargeting SSO control at normoxic (N) or hypoxic (H) conditions. Student's t test, *p<0.05, **p<0.01 and ***p<0.001. (C) Western blot of protein extracted from PANC-1 cells transfected with 20 nM of the HIF1A pseudoexon +10 SSO, a nontargeting SSO control and untransfected cells (UT) at normoxic or hypoxic conditions, using a HIF-1α specific antibody and a β-actin specific antibody as loading control. HIF-1α protein is not present at normoxia, but produced at hypoxic conditions in control. Translation of HIF-1α protein is reduced by the +10 SSO at hypoxic conditions.

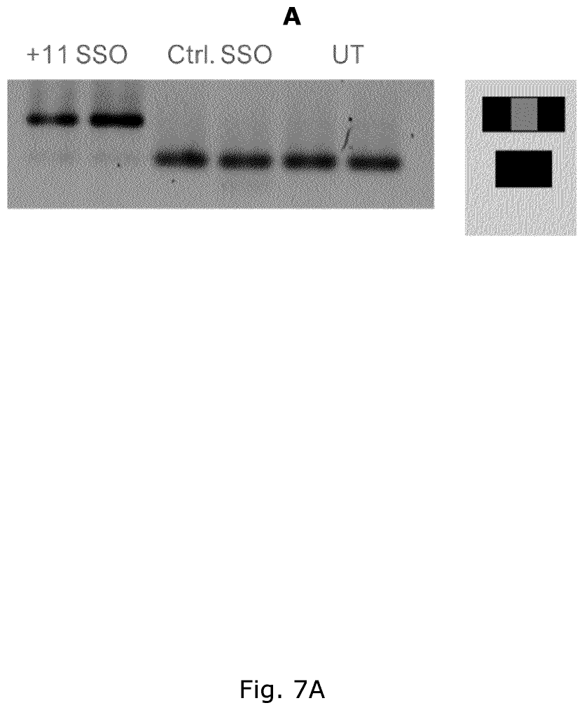

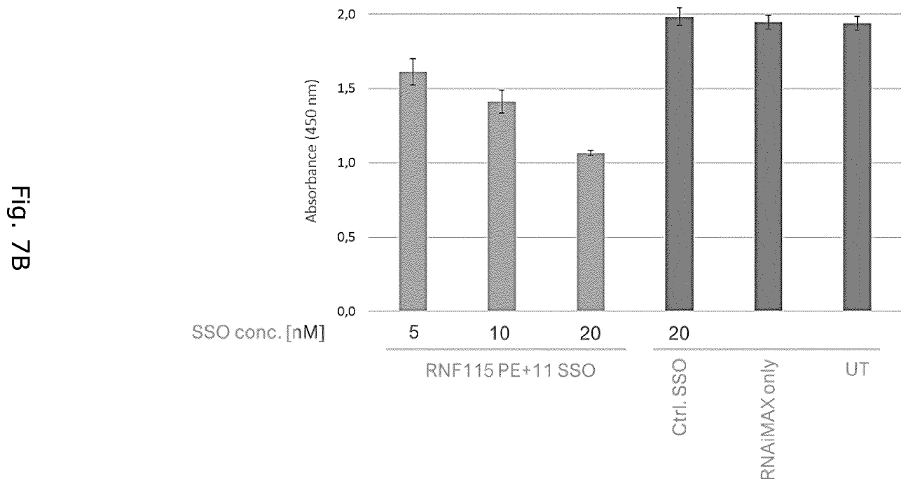

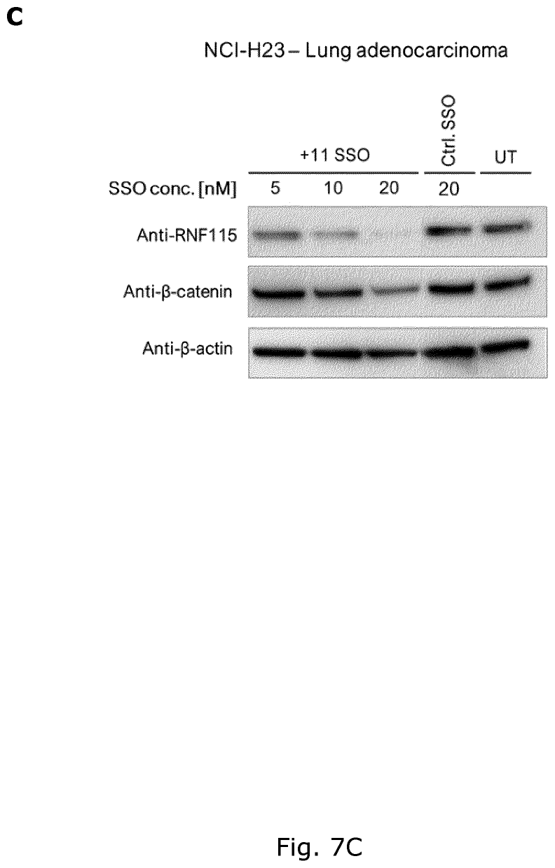

### FIG. 7

RNF115 pseudoexon inclusion leads to reduced RNF115 proteins levels and inhibits growth of lung adenocarcinoma cells. (A) RT-PCR analysis of RNF115 pseudoexon splicing in NCI-H23 cells transfected with the RNF115 pseudoexon +11 SSO and a nontargeting SSO control. The upper band includes the pseudoexon, which is activated by transfection of the +11 SSO. (B) WST-1 assay showing growth of NCI-H23 cells transfected with the RNF115+11 SSO and a nontargeting SSO control at different. The growth is inhibited by transfection of the +11 SSO in a dose-dependent manner. (C) Western blot of protein extracted from NCI-H23 cells transfected with 5, 10 and 20 nM of the RNF115 pseudoexon +11 SSO, a nontargeting SSO control and untransfected cells (UT), using a RNF115 specific antibody, a β-catenin antibody and a β-actin specific antibody as loading control. Protein levels of RNF115 and β-catenin are reduced by the +11 SSO.

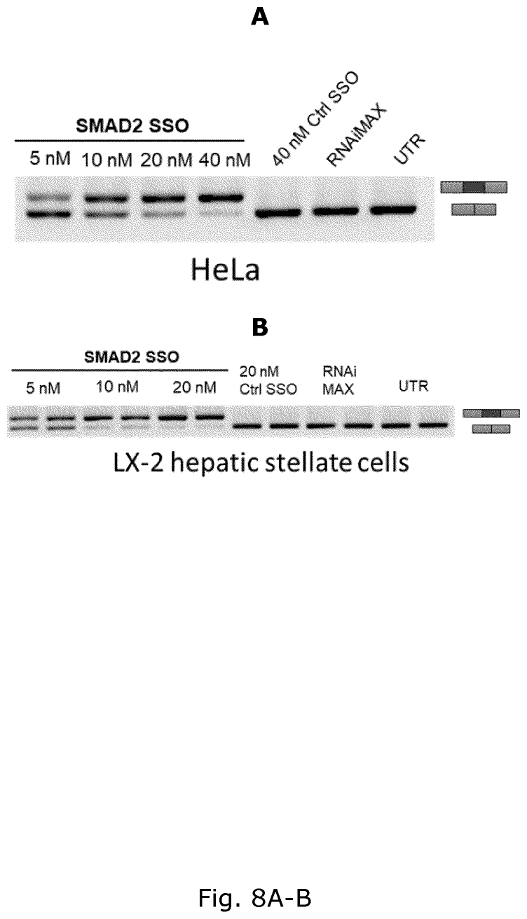

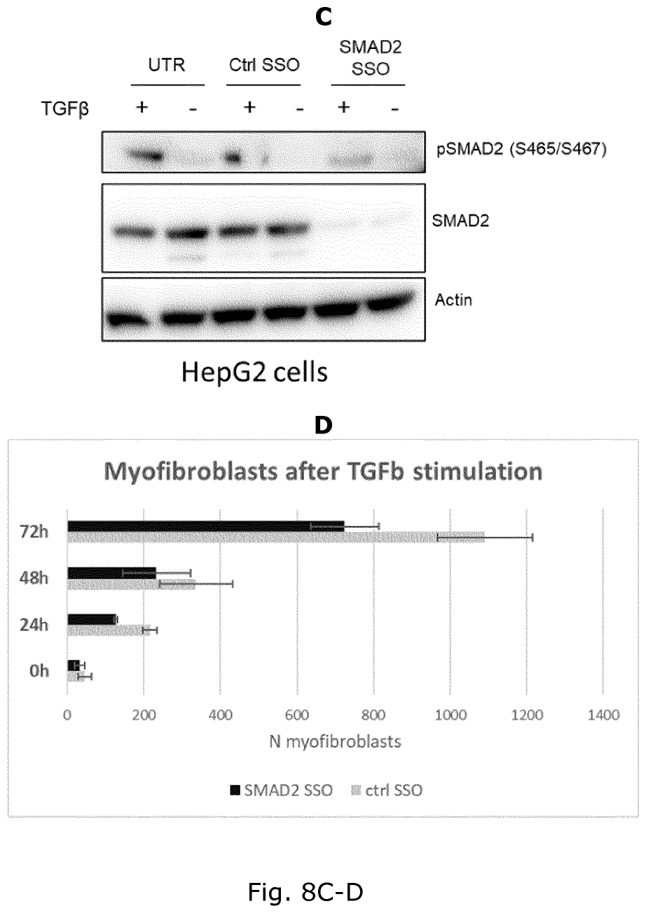

### FIG. 8

SMAD2 pseudoexon inclusion decreases fibrosis in hepatic stellate cells. (A) RT-PCR analysis of SMAD2 pseudoexon splicing in HeLa cells transfected with the SMAD2+11 SSO and a nontargeting SSO control. The upper band includes the pseudoexon which is activated by transfection with the +11 SSO. (B) RT-PCR analysis of SMAD2 pseudoexon splicing in LX-2 hepatic stellate cells transfected with the SMAD2+11 SSO and a nontargeting SSO control. (C) Western blotting analysis of protein from HepG2 liver cells transfected with the SMAD2 SSO, a nontargeting control SSO or untransfected (UTR) HepG2 cells, stimulated with (+) or without (−) TGFβ 16 hours before protein harvest. Transfection with the +11 SSO reduces SMAD2, as well as phosphoSMAD2 during TGFβ stimulation. (D) LX-2 hepatic stellate cells were transfected with either the SMAD2+11 SSO or a nontargeting SSO control and stimulated with TGFβ for 72 hours. Phase-contrast images were captured of the cells, and the number of differentiating cells were counted in ImageJ. Reduction of SMAD2 with the +11 SSO, decreases myofibroblast formation during TGFβ stimulation of fibrosis.

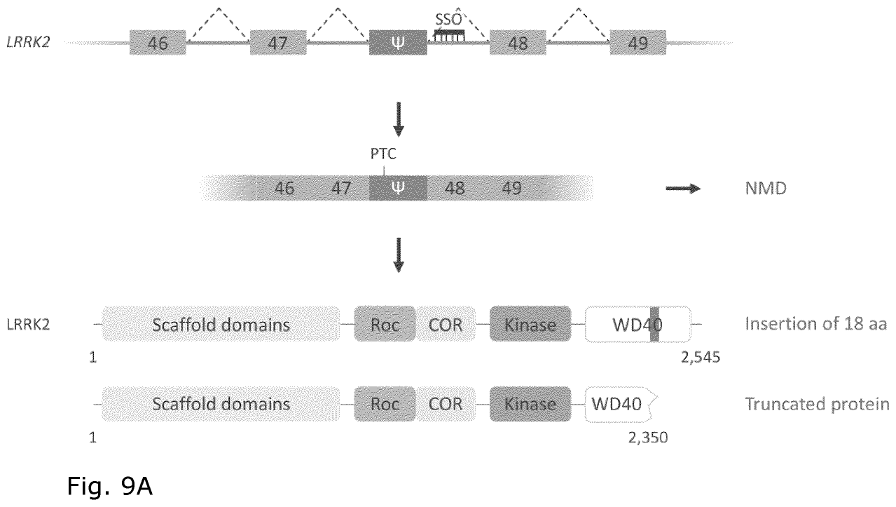

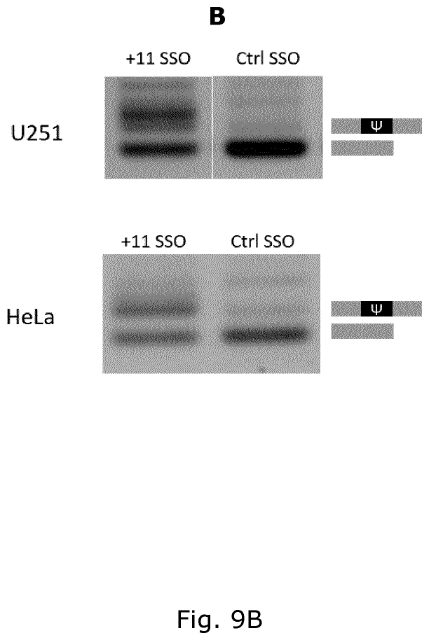

### FIG. 9

SSO-mediated LRRK2 pseudoexon inclusion. (A) Schematic representation of the consequences of induced LRRK2 pseudoexon inclusion. Inclusion of a 54 nt pseudoexon from LRRK2 intron 47 will introduce 18 amino acids to the WD40 domain of the translated LRRK2 protein, and inclusion of a 82 nt pseudoexon with the same 5′ splice site will cause a frame-shift and insertion of a premature termination codon which is a target for transcript degradation by nonsense-mediated mRNA decay (NMD). (B) RT-PCR analysis of LRRK2 pseudoexon splicing in HeLa and U251 cells transfected with the LRRK2 pseudoexon +11 SSO and a nontargeting SSO control (ctrl SSO). The upper bands include the pseudoexons, which is activated for inclusion in mRNA by transfection of the +11 SSO. UT; untransfected.

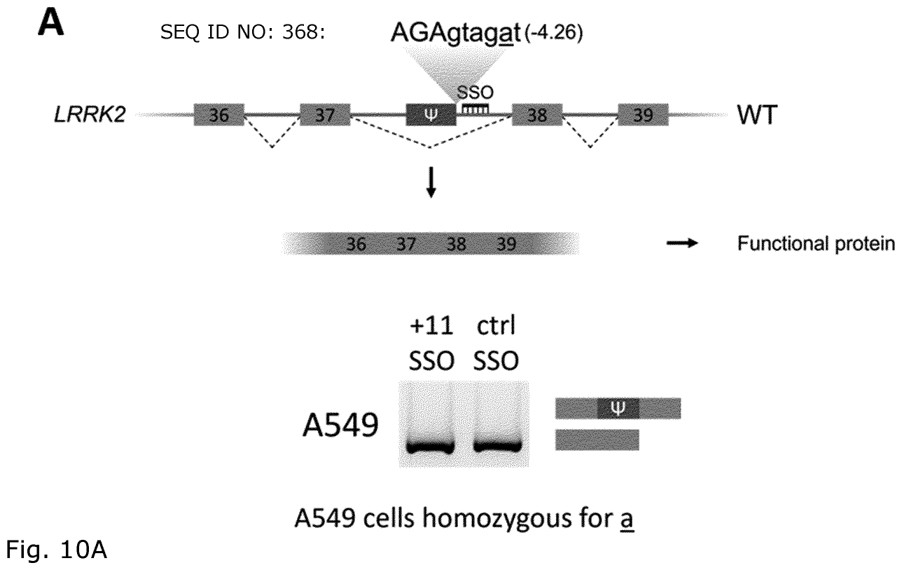

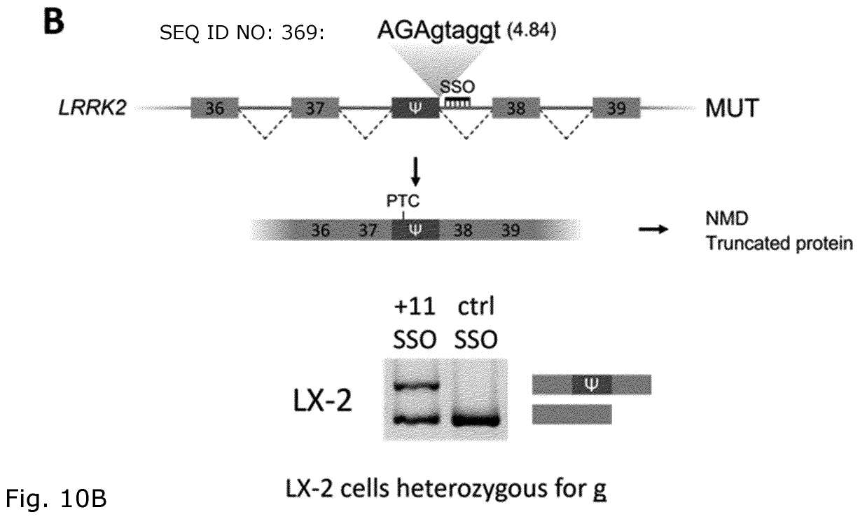

### FIG. 10

The possibility of SSO-mediated activation of the LRRK2 pseudoexon; chr12:40322690-40322887(+), depends on the presence of a common SNP; rs10878372 A/G, in the pseudoexon 5′ splice site, which enables G allele-specific activation of the pseudoexon. (A) Schematic representation of SSO treatment targeting the Sweet Spot region of the LRRK2 pseudoexon in the A allele. The pseudoexon 5′ splice site (AGAgtagat) (SEQ ID NO: 368) has a low MaxEnt score of −4.26. The SSO treatment will not induce pseudoexon inclusion from the A allele and a normal mRNA transcript is produced and can be translated into functional protein. This is demonstrated by RT-PCR analysis of LRRK2 pseudoexon splicing in A549 cells (homozygous for the A allele) transfected with the LRRK2 pseudoexon +11 SSO (5′-CAGACUACCAGACAUCUGACUAGAA-3′) (SEQ ID NO: 333) and a non-targeting SSO control (ctrl SSO) (5′-GCUCAAUAUGCUACUGCCAUGCUUG-3′) (SEQ ID NO: 126), in which no band with pseudoexon inclusion can be detected on the agarose gel. (B) Schematic representation of SSO treatment targeting the Sweet Spot region of the LRRK2 pseudoexon in the G allele. The pseudoexon 5′ splice site (AGAgtaggt) (SEQ ID NO: 369) has a high MaxEnt score of 4.84. The SSO treatment induces pseudoexon inclusion from the G allele and inclusion of the pseudoexon will introduce several in-frame premature termination codons (PTC) that will target the mRNA transcript for degradation by nonsense-mediated mRNA decay (NMD) and/or result in translation of truncated protein. This is demonstrated by RT-PCR analysis of LRRK2 pseudoexon splicing in LX-2 cells (heterozygous for the G allele) transfected with the LRRK2 pseudoexon+11 SSO (5′-CAGACUACCAGACAUCUGACUAGAA-3′) (SEQ ID NO: 333) and a non-targeting SSO control (ctrl SSO) (5′-GCUCAAUAUGCUACUGCCAUGCUUG-3′) (SEQ ID NO: 126), in which the upper band on the agarose gel represents inclusion of the pseudoexon. The G allele-specific pseudoexon activation allows downregulation of the hyperactivated LRRK2 disease allele with minimal or no effect on the wild type when the identified SNP (G) and dominant pathogenic mutation are located on the same allele. ψ; pseudoexon, WT; wild-type, MUT; mutant.

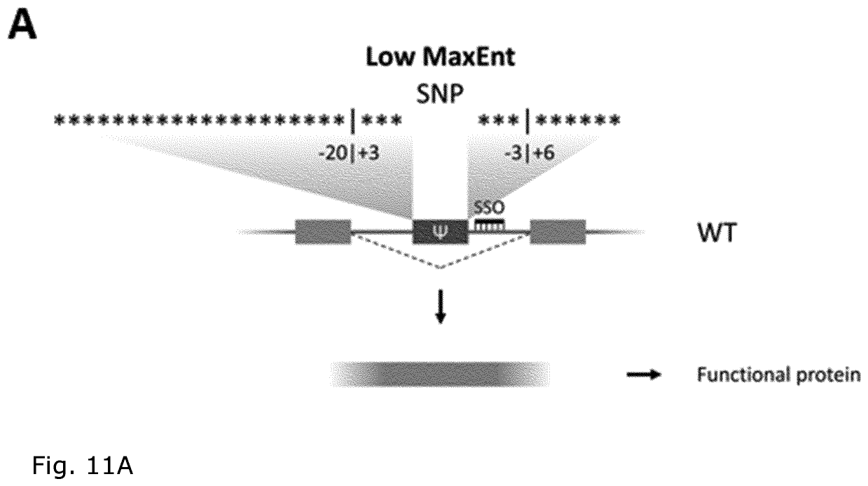

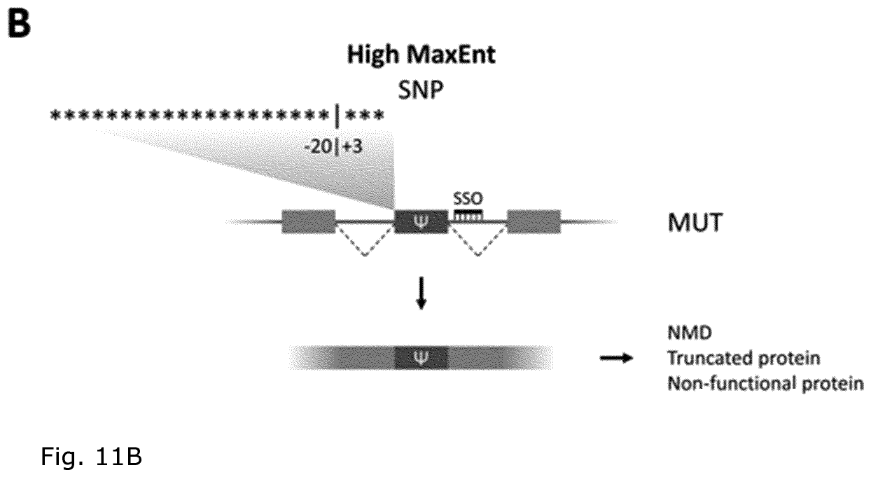

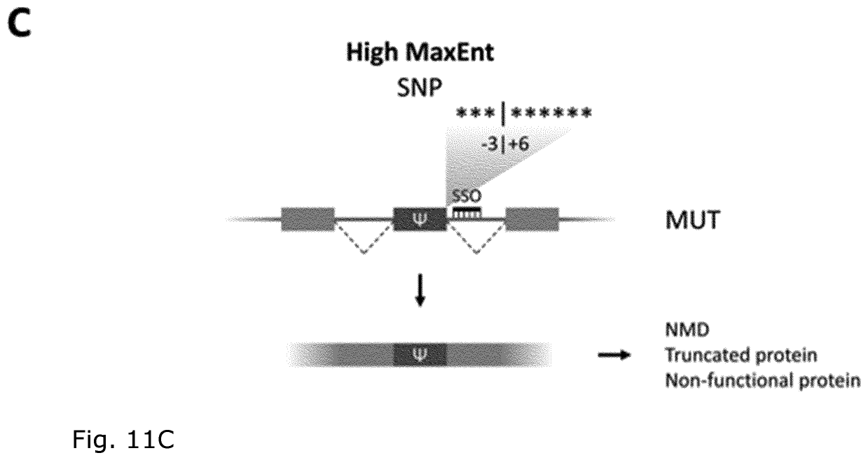

### FIG. 11

Pseudoexon activation mediated by an SSO targeting the Sweet Spot region (+9 to +39 nt downstream of the 5′ splice site) depends on the strength of the pseudoexon 3′ and 5′ splice sites. SNPs that are located in the 23-mer 3′ splice site (−20 to +3 nt of the intron-exon border) or in the 9-mer 5′ splice site (−3 to +6 nt of the exon-intron border) affects the strength of the given splice site (changes the MaxEnt score) and can be exploited for allele-specific pseudoexon activation, since such SNPs can make otherwise non-responding pseudoexons functional targets for SSO treatment and thereby allow for allele-specific inclusion or increased inclusion of the given pseudoexon in mature mRNA. (A) In alleles with a low MaxEnt score SNP variant in the pseudoexon 3′ or 5′ splice site, SSO-mediated activation of the pseudoexon is not possible or only possible to a low degree. Normal mRNA transcript is produced and can be translated into functional protein. (B) A SNP variant strengthens the pseudoexon 3′ splice site (increases the MaxEnt score) and enables SSO-mediated activation of the pseudoexon from alleles with the specific 3′ splice site high MaxEnt score SNP variant. (C) A SNP variant strengthens the pseudoexon 5′ splice site (increases the MaxEnt score) and enables SSO-mediated activation of the pseudoexon from alleles with the specific 5′ splice site high MaxEnt score SNP variant. In both cases, pseudoexon inclusion can target the mRNA transcript for degradation by nonsense-mediated mRNA decay (NMD) or result in translation of truncated, non-functional protein and will thereby modulate gene expression. Allele-specific pseudoexon activation in heterozygotes can allow downregulation of the disease allele with minimal or no effect on the wild type when the identified high MaxEnt score SNP variant and a pathogenic mutation are located on the same allele. The pseudoexon will be activated from both alleles in homozygotes for the high MaxEnt score SNP variant. * indicates possible location of SNPs affecting the strength of the pseudoexon 3′ or 5′ splice site. ψ; pseudoexon, WT; wild-type, MUT; mutant.

The present invention will now be described in more detail in the following.

### DETAILED DESCRIPTION OF THE INVENTION

### Definitions

Prior to discussing the present invention in further details, the following terms and conventions will first be defined:

### Pseudoexon (PE)

In the present context, the terms “pseudoexon” or “PE” relate to exonic-like sequences that are present within intronic regions but are normally ignored by the spliceosomal machinery. Thus, pseudoexons do not, under normal splicing conditions, become part of the mature mRNA or only become part of the mature mRNA at low levels. Thus, pseudoexons are intronic sequences flanked by 3′ and 5′ splice sites, but pseudoexons are often not annotated due to the normally low inclusion into the mRNA transcript. Moreover, when included, pseudoexons will either disrupt or significantly alter the function of the normal transcript or protein.

### Function-Disabling Pseudoexon

In the present context, the term “function-disabling pseudoexon”, relates to the situation that the presence of the pseudoexon in the mature mRNA results in inactivation, reduced activity, reduced transcription and/or altered function of the protein expressed from the mRNA (compared to mature mRNA without the pseudoexon).

### Exonic Splicing Enhancer (ESE)

As used herein, the terms “Exonic Splicing Enhancer” or “Exon Splicing Enhancer” or “ESE” means a nucleotide sequence, which when present in the exon and accessible for binding of nuclear splicing regulatory proteins and/or by forming a secondary structure or a part thereof of the pre-mRNA stimulates inclusion of this exon into the final spliced mRNA during pre-mRNA splicing.

### Exonic Splicing Silencer (ESS)

As used herein, the terms “Exonic Splicing Silencer” or “Exon Splicing Silencer” or “ESS” mean a nucleotide sequence, which when present in the exon and accessible for binding of nuclear splicing regulatory proteins and/or by forming a secondary structure or a part thereof of the pre-mRNA inhibits inclusion of this exon into the final spliced mRNA during pre-mRNA splicing.

### Intronic Splicing Enhancer (ISE)

As used herein, the terms “Intronic Splicing Enhancer” or “Intron Splicing Enhancer” or “ISE” mean a nucleotide sequence, which when present in the intron and accessible for binding of nuclear splicing regulatory proteins and/or by forming a secondary structure or a part thereof of the pre-mRNA stimulates inclusion of an exon into the final spliced mRNA during pre-mRNA splicing.

### Intronic Splicing Silencer (ISS)

As used herein, the terms “Intronic Splicing Silencer” or “Intron Splicing Silencer” or “ISS” mean a nucleotide sequence, which when present in the intron and accessible for binding of nuclear splicing regulatory proteins and/or by forming a secondary structure or a part thereof of the pre-mRNA inhibits inclusion of an exon into the final spliced mRNA during pre-mRNA splicing.

### Splice Sites

Splice sites at the 5′ end of the intron are often called the “5′ splice site,” or “splice donor site” and the splice site at the 3′ end of the intron are often called the “3′ splice site” or “splice acceptor site”.

Nonsense-Mediated mRNA Decay (NMD)

“Nonsense-mediated mRNA decay” or “NMD” is a surveillance pathway that exists in all eukaryotes. Its main function is to reduce errors in gene expression by eliminating mRNA transcripts that contain premature termination codons (PTCs). In relation to the present invention, the introduction of the pseudoexon may induce NMD if a PTC is present in the introduced pseudoexon or when the pseudoexon changes the reading frame of the mature transcript.

### Nucleotide

As used herein, “nucleotide” means a nucleoside further comprising a phosphate linking group. As used herein, “linked nucleosides” may or may not be linked by phosphate linkages and thus includes, but is not limited to “linked nucleotides”. As used herein, “linked nucleosides” are nucleosides that are connected in a continuous sequence (i.e., no additional nucleosides are present between those that are linked).

### Nucleobase

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

### Unmodified Nucleobase

As used herein the terms, “unmodified nucleobase” or “naturally occurring nucleobase” mean the naturally occurring heterocyclic nucleobases of RNA or DNA: the purine bases adenine (A) and guanine (G), and the pyrimidine bases thymine (T), cytosine (C) (including 5-methyl C), and uracil (U).

### Modified Nucleobase

As used herein, “modified nucleobase” means any nucleobase that is not a naturally occurring nucleobase.

### Modified Nucleoside

As used herein, “modified nucleoside” means a nucleoside comprising at least one chemical modification compared to naturally occurring RNA or DNA nucleosides. Modified nucleosides comprise a modified sugar moiety and/or a modified nucleobase.

Constrained Ethyl Nucleoside (cEt)

As used herein, “constrained ethyl nucleoside” or “cEt” means a nucleoside comprising a bicyclic sugar moiety comprising a 4′-CH(CH3)-0-2′bridge.

### Locked Nucleic Acid Nucleoside (LNA)

As used herein, “locked nucleic acid nucleoside” or “LNA” means a nucleoside comprising a bicyclic sugar moiety comprising a 4′-CH2-0-2′bridge.

2′-substituted Nucleoside

As used herein, “2′-substituted nucleoside” means a nucleoside comprising a substituent at the 2′-position other than H or OH. Unless otherwise indicated, a 2′-substituted nucleoside is not a bicyclic nucleoside.

2′-deoxynucleoside

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

### Oligonucleotide

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

### Terminal Group

As used herein, “terminal group” means one or more atoms attached to either, or both, the 3′ end or the 5′ end of an oligonucleotide. In certain embodiments a terminal group is a conjugate group. In certain embodiments, a terminal group comprises one or more terminal group nucleosides.

### Conjugate

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

### Conjugate Linking Group

As used herein, “conjugate linking group” means any atom or group of atoms used to attach a conjugate to an oligonucleotide or oligomeric compound.

### Splice Switching Oligonucleotide (SSO)

As used herein, “splice switching oligonucleotide” or “SSO” means a compound comprising or consisting of an oligonucleotide at least a portion of which is complementary to a target nucleic acid to which it is capable of hybridizing, resulting in at least one change in the splicing pattern of the targeted pre-mRNA.

A splice switching oligonucleotide could also be termed splice switching antisense oligomer (SSO).

mRNA

As used herein, “mRNA” means an RNA molecule that encodes a protein.

### Pre-mRNA

As used herein, “pre-mRNA” means an RNA transcript that has not been fully processed into mRNA. Pre-mRNA includes one or more introns.

Target pre-mRNA

As used herein, the term “target pre-mRNA” means a nucleic acid molecule to which an SSO hybridizes.

### Change in the Splicing Pattern of the Targeted Pre-mRNA

As used herein, “a change in the splicing pattern of the targeted pre-mRNA” means a change in the pre-mRNA splicing process resulting in insertion of a proportion, for instance corresponding to a pseudoexon or a proportion thereof, into the produced mRNA when compared to the reference nucleotide sequence of the targeted pre-mRNA.

### Transcript

As used herein, “transcript” means an RNA molecule transcribed from DNA. Transcripts include, but are not limited to mRNA, pre-mRNA, and partially processed RNA.

### Targeting and Targeted to

As used herein, “targeting” or “targeted to” means the association of an SSO to a particular target nucleic acid molecule or a particular region of a target nucleic acid molecule. An SSO targets a target nucleic acid if it is sufficiently complementary to the target nucleic acid to allow hybridization under physiological conditions.

### Nucleobase Complementarity and Complementarity

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an SSO that is capable of base pairing with a nucleobase of its target nucleic acid. For example, if a nucleobase at a certain position of an SSO is capable of hydrogen bonding with a nucleobase at a certain position of a target nucleic acid, then the position of hydrogen bonding between the oligonucleotide and the target nucleic acid is considered complementary at that nucleobase pair. Nucleobases comprising certain modifications may maintain the ability to pair with a counterpart nucleobase and thus, are still capable of nucleobase complementarity.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary. In another embodiment, the oligomeric compounds comprise up to 3 mismatches, such as up to 2 or 1 mismatches. Preferably, no mismatches are present.

### Hybridization

As used herein, “hybridization” means the pairing of complementary oligomeric compounds (e.g., an SSO and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

### Motif

As used herein, “motif” means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

### Type of Modification

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

### Differently Modified

As used herein, “differently modified” means chemical modifications or chemical substituents that are different from one another, including absence of modifications. Thus, for example, an MOE nucleoside and an unmodified DNA nucleoside are “differently modified,” even though the DNA nucleoside is unmodified. Likewise, DNA and RNA are “differently modified,” even though both are naturally occurring unmodified nucleosides. Nucleosides that are the same but comprise different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

### MaxEnt Score

The MaxEnt score is a score known to the skilled person that accounts for adjacent as well as non-adjacent dependencies between positions within the splice site, using a maximum entropy principle to identify optimal splice sites. A high score indicates a high probability of a functionally strong splice site, but splice sites with lower scores may be functional through activation by splicing factors bound to the pre-mRNA at ESE or ISE motifs. Likewise, a splice site with a high score may be functionally repressed by nearby or overlapping ESS or ISS motifs binding inhibitory splicing factors.

MaxEnt score according to the present invention is determined using the program “MaxEntScan” version 20 Apr. 2004. The same software can be used to determine:
5′ splice site scores: (http://hollywood.mit.edu/burqelab/maxent/Xmaxentscan scoreseq.html); and
3′ splice site scores: (http://hollywood.mit.edu/burqelab/maxent/Xmaxentscan scoreseq acc.ht ml)

Determination of MaxEnt score is further described in Gene Yeo and Christopher B Burge (J Comput Biol. 2004; 11(2-3):377-94).

The MaxEnt score is a specific value which can only be determined in one way.

Albeit the inclusion of MaxEnt scores significantly improves the overall predictability and is preferred, in specific embodiments of the invention, the MaxEnt score is optional.

### Pseudoexon Identification

Pseudoexons are identified with precise genomic coordinates of the 3′ splice site and the 5′ splice site using a double-junction approach. In this approach, RNA sequencing fragments are filtered to retain only those with evidence of at least two splicing junctions. Exon coordinates can be extracted from the mapped reads, allowing for a gap of a certain length in the middle of the fragment where there is no direct sequence. The exons which are supported by a splicing junction at both ends in the same fragment are classified by comparing to a known gene annotation, and novel pseudoexons can be identified as exons that overlap introns, but not any existing exons.

The “Sweet Spot region” is defined as the region from +9 to +39 downstream of the 5′ splice site of a pseudoexon, both positions included. Pseudoexons that can be activated by an SSO binding to a region within the Sweet Spot region is identified by the following parameters:

### Pseudoexon Parameters/Criteria:

Pseudoexon length<160 nt;
Pseudoexon length>30 nt;
The last 3 nt of the pseudoexon are different from TAG;
Donor splice site has a MaxEnt score 4.33;
Donor splice site has a MaxEnt score 10.06; and
Acceptor splice site has a MaxEnt score 3.63;

### Sequence Region+9 to +39 Downstream to the 5′ Splice Site of Said Pseudoexon Parameters/Criteria:

Total pyrimidines≤20;
Total thymidine bases≤12;
Total thymidine bases≥4;
Total guanine bases≤12;
Maximum length of thymidine polymer≤4;
Maximum length of pyrimidine polymer≤10;
Minimum length of purine polymer≥3; and
Maximum number of guanine polymers of at least 3 nt length≤2.

In an RNA sequence, the presence of a uracil may be considered equivalent to the presence of a thymidine at the same position in the corresponding DNA sequence. As such, the presence of a thymidine in the DNA sequence may also be considered equivalent to a uracil in the same position in the corresponding RNA sequence. The criteria covering sequences with thymidines and pyrimidines are therefore identical to equivalent criteria for sequences with uracil and pyrimidines when the analyzed sequence is an RNA sequence.

### Identifying Sequences and Producing SSOs

As outlined above the present invention relates to the identification of sequence parameters in a gene comprising a pseudoexon, which can be used to determine if it is possible (with high probability) to get the pseudoexon incorporated in the mature mRNA using a splice switching oligonucleotide (SSO) (see also example 1 and corresponding figures). Thus, an aspect of the invention relates to a method for identifying SSOs able to modulate expression of a target protein in a cell by promoting incorporation of a pseudoexon into the mature mRNA upon binding to the pre-mRNA in the region +9 to +39 downstream to the 5′ splice site of said pseudoexon, the method comprising;
a) providing one or more gene sequences comprising one or more identified pseudoexons;
b) determining for the one or more gene sequences; if the pseudoexon meets the following criteria:
   a pseudoexon length<160 nt;
   a pseudoexon length>30 nt;
   the last 3 nt of the pseudoexon are different from TAG;
   the donor splice site has a MaxEnt score 4.33;
   the donor splice site has a MaxEnt score 10.06; and
   the acceptor splice site has a MaxEnt score 3.63;
c) determining for the one or more gene sequences if the sequence region +9 to +39 downstream to the 5′ splice site of said pseudoexon (3) meets the following criteria:
   total pyrimidines≤20;
   total thymidine bases≤12;
   total thymidine bases>=4;
   total guanine bases≤12;
   maximum length of thymidine polymer≤4;
   maximum length of pyrimidine polymer≤10;
   maximum length of purine polymer≥3; and
   maximum number of guanine polymers of at least 3 nt length≤2.
      wherein, if one or more gene sequences meet the criteria according to point b) and point c), said region +9 to +39 downstream to the 5′ splice site of said pseudoexon is considered a target for an SSO able to, in vivo, hybridize to the pre-mRNA (2) of said gene within the region +9 to +39 downstream to the 5′ splice site of said pseudoexon (3); and resulting in said pseudoexon (3) becoming part of the mature mRNA to a greater extent compared to corresponding pre-mRNA not contacted with the SSO (1);
      and
      optionally, producing said SSO, optionally for use as a medicament.

As outlined in example 2, the included selection parameters (criteria) can discriminate between genes where SSOs can be used (Table 1 in example 2) and genes where the SSOs will not be able to incorporate the pseudoexon (Table 2 in example 2).

In an embodiment, the invention is computer-implemented (except for the optional step of producing said SSO, optionally for use as a medicament).

In another embodiment, the one or more gene sequences comprising one or more identified pseudoexons are provided in the form of a database or on another digital storage mean.

In an embodiment, the one or more gene sequences comprising one or more identified pseudoexons are gene sequences, which contains disease-causing genes, such as genes encoding dominant negative proteins, such as characterized by increased expression or altered function of the gene, or genes.

In another embodiment the pseudoexons is in a gene where decreased level of normal functional gene product has a therapeutic benefit, such as genes associated with cancer, diabetes, inflammation, neurodegenerative or neurological disorders, tissue degeneration, tissue fibrosis and chirosis, metabolic conditions, chronic liver disease and inherited retinal dystrophies (IRDs).

In yet another embodiment, the one or more gene sequences comprising one or more identified pseudoexons are gene sequences, which contains disease-causing genes, such as genes encoding proteins causing/enhancing/influencing diseases such as cancer, diabetes, inflammation, neurodegenerative or neurological disorders, tissue degeneration, tissue fibrosis and chirosis, metabolic conditions, chronic liver disease and inherited retinal dystrophies (IRDs). The disease may be due to enhanced expression.

In yet an embodiment, the one or more gene sequences comprising one or more identified pseudoexons are gene sequences, which are not only known to cause inherited disease(s).

In a preferred embodiment, SSOs are produced against an identified region +9 to +39 downstream to the 5′ splice site of said pseudoexon, which region meets the above outlined criteria. Examples 3-10 show specific examples of the effect of SSOs against identified target sequences.

In an embodiment, said produced SSO comprises a sequence, which is complementary or substantially complementary to a region +9 to +39 downstream to the 5′ splice site of said pseudoexon (3), such as within the region +11 to +35 downstream to the 5′ splice site. In the example section (examples 2-8), SSOs which are complementary to position +11 to +35 have been used (25 nt long). In examples 9 and 10 specific optimization of the target region for the SSO has been further optimized for the two genes HIF1A and TRPM7. In examples 3 and 13 specific optimization of the target region for the SSO has been further optimized for the two genes SMAD2 and LRRK2.

In another embodiment, said produced SSO comprises a sequence which is substantially complementary to the region +9 to +39 downstream to the 5′ splice site of said pseudoexon (3), and comprises at the most 3 mismatches, such as at the most 2 mismatches or such as at the most 1 mismatch.

In an embodiment, said produced SSO comprises a sequence which is complementary to a region +9 to +39 downstream to the 5′ splice site of said pseudoexon (3) such as within the region +11 to +35 downstream to the 5′ splice site. In the examples, SSOs targeting position +11 to +35 were tested.

In an embodiment, the complementary region being in the range 9-31 nucleotides, such as 15-30, such as 15-25 or such as 9-15, or such as 15-30, such as 20-25. If e.g. LNA are used or other high-binding nucleotides, the length of the SSO may be in the shorter ranges.

The SSOs may be able to modulate expression of the target protein in different ways. Thus, in an embodiment, hybridization of the SSO to the pre-mRNA in vivo results in:
decreasing the level of mRNA encoding the functional protein (4); and/or
decreasing expression of the functional protein (6); and/or
loss of function of the functional protein (5); and/or
a new function of the functional protein (6); and/or
mis-localization of the protein.
mis-localization of the mRNA encoding the functional protein.

In yet an embodiment, the one or more gene sequences from step a) causes a disorder or condition characterized by increased expression or altered function of the gene. In an embodiment, the disorder or condition is an autosomal dominant negative disorder.

In yet an embodiment, the one or more gene sequences from step a) is therapeutically beneficial when the level of normal functional gene product is decreased. In an embodiment, the disorder or condition is not directly associated with a disease-causing gene.

In an embodiment, said SSO has a length in the range 9-100 nucleotides, such as 9-50 nucleotides, preferably in the range 9-40 nucleotides and more preferably in the range 15-31 nucleotides or 15-25 nucleotides.

In an embodiment, said SSO comprises a sequence which is complementary or substantially complementary to a polynucleotide in the pre-mRNA, wherein said sequence has a length in the range 9-31 nucleotides, such as 15-25 nucleotides, preferably the sequence is complementary at a range of 9-31 nucleotides, such as 9-20, or such as 20-31 nucleotides, such as 25-31 nucleotides.

In a preferred embodiment, said produced SSO comprises one or more artificial nucleotides, such as sugar-modified nucleotides.

In another preferred embodiment, the SSO does not mediate RNAse H mediated degradation of the mRNA in vivo.

In an embodiment, at least one modified sugar moiety is a 2′-substituted sugar moiety.

In an embodiment, said 2′-substituted sugar moiety has a 2′-substitution selected from the group consisting of 2′-O-Methyl (2′-OMe), 2′-fluoro (2′-F), and 2′-O-methoxyethyl (2′-MOE).

In an embodiment, said 2′-substitution of said at least one 2′-substituted sugar moiety is a 2′-O-methoxyethyl (2′-MOE).

In an embodiment, at least one modified sugar moiety is a bicyclic sugar moiety. In an embodiment, at least one bicyclic sugar moiety is a locked nucleic acid (LNA) or constrained ethyl (cEt) nucleoside.

In an embodiment, at least one sugar moiety is a sugar surrogate.

In an embodiment, at least one sugar surrogate is a morpholino.

In an embodiment, at least one morpholino is a modified morpholino.

In an embodiment, the SSO comprises at least one internucleoside N3′ to P5′ phosphoramidate diester linkage.

In an embodiment, the modified oligonucleotide comprises at least one internucleoside phosphorothioate linkage.

In an embodiment, all internucleoside linkages are phosphorothioate.

In an embodiment, the SSO is conjugated to delivery elements, such as selected from the group consisting of Gal-Nac, (poly-)unsaturated fatty acids (such as oleoyl and linolenoyl), anisamide, anandamide, folic acid (FolA), carbachol, estrone, Retro-1, phospholipids, α-tocopherol (α-TP), cholesterol, squalene (SQ), unbranched fatty acids (such as lauroyl, myristoyl, palmitoyl, stearoyl, and docosanoyl), and cell penetrating peptides.

In an embodiment, the one or more gene sequences comprising one or more identified pseudoexons are involved in a disease or disorder selected from the group consisting of cancer, Inflammatory diseases, Neurodegenerative or neurological diseases, Metabolic conditions, Chronic liver disease and Inherited retinal dystrophies (IRDs).

In an embodiment, the Chronic liver disease is nonalcoholic fatty liver disease.

In an embodiment, said cancer is selected from the group consisting of, brain cancer, glioblastoma, lung cancer, colorectal cancer, skin cancer, pancreas cancer, bladder cancer, liver cancer, breast cancer, eye cancer and prostate cancer.

In yet an embodiment, said cancer is a haematological cancer, such as selected from the group consisting of multiple myeloma, acute myeloblastic leukemia, chronic myelogenic leukemia, acute lymphoblastic leukemia and chronic lymphocytic leukemia.

As outlined in the example section, the inventing team has identified a number of clinical relevant genes comprising pseudoexons, which can be incorporated in the mature mRNA, thereby inactivating/inhibiting/altering the function of the expressed (disease-causing) protein).

Thus, in an embodiment, said SSO is complementary or substantially complementary to region within a nucleic acid selected from the group consisting of
a nucleic acid according to any of any of SEQ ID NO's: 106 (RNF115), 1-26, 79-105, 107-125 and 137-201; or
a nucleic acid comprising 1 or 2 or 3 substitutions when compared to any of any of SEQ ID NO's: 106, 1-26, 79-105, 107-125 and 137-201; or
a nucleic acid sequence having at least 90% sequence identity to any of any of SEQ ID NO's: 106, 1-26, 79-105, 107-125 and 137-201.

In a preferred embodiment, said produced SSO is complementary or substantially complementary to region within a nucleic acid selected from the group consisting of
a nucleic acid according to any of SEQ ID NO's: 79-100; or
a nucleic acid comprising 1 or 2 or 3 substitutions when compared to any of SEQ ID NO's: 79-100; or
a nucleic acid sequence having at least 90% sequence identity to any of SEQ ID NO's: 79-100.

In a more preferred embodiment, said produced SSO is complementary or substantially complementary to region within a nucleic acid selected from the group consisting of
a nucleic acid according to any of SEQ ID NO's: 79-85; or
a nucleic acid comprising 1 or 2 or 3 substitutions when compared to any of SEQ ID NO's: 79-85; or
a nucleic acid sequence having at least 90% sequence identity to any of SEQ ID NO's: 79-85.

Based on the provided gene sequence data in the example section, the skilled person could easily translate this information into specific SSO sequences, e.g. by using the sections underlined in the tables and designing SSOs complementary thereto.

In an embodiment, the SSO is selected from the group consisting of:
a nucleic acid according to any of SEQ ID NO's: 127-136; or
a nucleic acid comprising 1 or 2 or 3 substitutions when compared to any of SEQ ID NO's: 127-136; or
a nucleic acid sequence having at least 90% sequence identity to any of SEQ ID NO's: 127-136.

Preferably, the nucleic acid is SEQ ID NO: 127 or 128 or 133 or 136. As shown in examples 9 and 10, these SSOs have been optimized within the Sweet Spot region. Thus, by moving the binding region e.g. just 1 or 2 positions the efficiency can surprisingly be increased even further. More preferably, the nucleic acid is SEQ ID NO: 128 or 136.

In another preferred embodiment, the SSO is complementary or substantially complementary to region within a nucleic acid selected from the group consisting of
a nucleic acid according to any of SEQ ID NO: 21 (targeting SMAD2), SEQ ID NO: 106 (targeting RNF115) and any of SEQ ID No's: 141, 142, 158, and 159 (targeting LRRK2);
a nucleic acid comprising 1 or 2 or 3 substitutions when compared to any of SEQ ID NO's: 21, 106, 141, 142, 158, and 159; or
a nucleic acid sequence having at least 90% sequence identity to any of SEQ ID NO's: 21, 106, 141, 142, 158, and 159.

Example 3 shows data on SMAD2 targeting (see also example 11).

Example 12 shows data in RNF115 targeting.

Example 13 shows data on LRRK2 targeting, including allele specific targeting.

In yet another preferred embodiment, the SSO is complementary or substantially complementary to region within a nucleic acid selected from the group consisting of
a nucleic acid according to any of SEQ ID NO: 100, 114, 118, 150 and 151;
a nucleic acid comprising 1 or 2 or 3 substitutions when compared to any of SEQ ID NO's: 100, 114, 118, 150 and 151; or
a nucleic acid sequence having at least 90% sequence identity to any of SEQ ID NO's: 100, 114, 118, 150 and 151.

In yet another preferred embodiment, the SSO is complementary or substantially complementary to region within a nucleic acid selected from the group consisting of
a nucleic acid according to any of SEQ ID NO: 12, 24 or 26;
a nucleic acid comprising 1 or 2 or 3 substitutions when compared to any of SEQ ID NO's: 12, 24 or 26; or
a nucleic acid sequence having at least 90% sequence identity to any of SEQ ID NO's: 12, 24 or 26.

As outlined in the example section, different genes have already been targeted using the selection criteria according to the invention (Example 2, Table 1) or has been identified as targeting sequences using the selection criteria according to the invention (Example 8, Table 3, Example 11, tables 6-7). Thus, in an embodiment, the produced SSO is complementary or substantially complementary to a SEQ ID NO as outlined in Table 1, Table 3, Table 6 and Table 7 (Sweet Spot region). The genes listed in Table 1 or Table 3 or Table 6-7, may be specifically preferred in relation to certain diseases, as outlined below. Again, Sweet Spots for the SSOs are outlined in Table 1 and Table 3 and Tables 6-7. Table 2 shows pseudoexon sequences for which the criteria according to the invention is not fulfilled and which are not functional sites for SSOs.

### Cancer Out of Frame (NMD):

The following genes could be relevant to target in relation to cancer treatment: TXNRD1, SLC7A11, STAT5B, MAPKAPK5, ZYG11A, ROCK1, MCCC2, SMYD2, DIAPH3, COPS3, SNX5, YBX1, CHD1L, PTPN11, UBAP2L, RNF115, HGS, TLK1, WWTR1, HMGCS1, SND1, THOC2, ORC1, TAF2, HIF1A, TRPM7, CPPS1, LRP6, MELK, TTBK2, TTK, ITGBL1, ROCK2, TASP1, FLT1, KNTC1, SMC1A, ZNF558, PMPCB and DBI.

Thus, in an embodiment, the produced SSO is complementary or substantially complementary to a SEQ ID NO as outlined in Table 1 and Table 3 and Tables 6-7, wherein the gene is selected from the group consisting of TXNRD1, SLC7A11, STAT5B, MAPKAPK5, ZYG11A, ROCK1, MCCC2, SMYD2, DIAPH3, COPS3, SNX5, YBX1, CHD1L, PTPN11, UBAP2L, RNF115, HGS, TLK1, WWTR1, HMGCS1, SND1, THOC2, ORC1, TAF2, HIF1A, TRPM7, CPPS1, LRP6, MELK, TTBK2, TTK, ITGBL1, ROCK2, TASP1, FLT1, KNTC1, SMC1A, ZNF558, PMPCB and DBI; for use in the treatment of cancer. In a preferred embodiment the gene is RNF115 (see example 12).

### Cancer in Frame:

The following genes could be relevant to target in relation to cancer treatment: ROCK1, E2F3, LRIG2, HSPG2, SLC2A1, KNTC1, DIAPH3, FDFT1, THOC2 and SMC1A, DDR2, STAG2, TRPM7, LINGO2, RAP1GDS, BUD1, CD44, CDKL5, RNF115, UBAP2L, ZNF558, RBPJ, EFEMP1, and FLT1.

Thus, in an embodiment, the produced SSO is complementary or substantially complementary to a SEQ ID NO as outlined in Table 1 and Table 3 and Tables 6-7, wherein the gene is selected from the group consisting of ROCK1, E2F3, LRIG2, HSPG2, SLC2A1, KNTC1, DIAPH3, FDFT1, THOC2, DDR2, STAG2, TRPM7, LINGO2, SMC1A, RAP1GDS, BUD1, CD44, CDKL5, RNF115, UBAP2L, ZNF558, RBPJ, EFEMP1, and FLT1; for use in the treatment of cancer.

### Neurological Disease (Out of Frame):

The following genes could be relevant to target in relation to Neurological diseases: ROCK1, HTT, OGA, TMEM97, PICALM, LRRK2, UBAP2L, SMC1A, TTBK2.

Thus, in an embodiment, the produced SSO is complementary or substantially complementary to a SEQ ID NO as outlined in Table 1 and Table 3 and Tables 6-7, wherein the gene is selected from the group consisting of ROCK1, HTT, OGA, TMEM97, PICALM, LRRK2, UBAP2L, SMC1A and TTBK2; for use in the treatment of a neurological disease. In a preferred embodiment the gene is LRRK2 (see example 13).

In an even more preferred embodiment, the neurological disease is selected from the group consisting of Alzheimer's disease, Amyotrophic lateral sclerosis, Friedreich's ataxia, Huntington's disease, Lewy body disease, Parkinson's disease and Spinal muscular atrophy.

Neurodegeneration is the progressive loss of structure or function of neurons, including death of neurons. Many neurodegenerative diseases—including amyotrophic lateral sclerosis, Parkinson's disease, Alzheimer's disease, Huntington's disease, and prion diseases—occur as a result of neurodegenerative processes.

### Neurological Disease (in Frame):

The following genes could be relevant to target in relation to Neurological diseases: ROCK1, E2F3, SLC2A13, ASIC1, TRPM7, LINGO2, LRIG2, LRRK2, UBAP2L, SMC1A, ATXN7, and CLCN1.

Thus, in an embodiment, the produced SSO is complementary or substantially complementary to a SEQ ID NO as outlined in Table 1 and Table 3 and Tables 6-7, wherein the gene is selected from the group consisting of ROCK1, E2F3, SLC2A13, TRPM7, LINGO2, ASIC1, LRIG2, LRRK2, UBAP2L, SMC1A, ATXN7, and CLCN1; for use in the treatment of a neurological disease.

Again, in an even more preferred embodiment, the neurological disease is selected from the group consisting of Alzheimer's disease, Amyotrophic lateral sclerosis, Friedreich's ataxia, Huntington's disease, Lewy body disease, Parkinson's disease and Spinal muscular atrophy.

### Diabetes:

The following genes could be relevant to target in relation to diabetes: TXNRD1 (Diabetes), DYRK1A (Diabetes) TRPM7 (Diabetes) and PHLPP1 (Diabetes and obesity).

Thus, in an embodiment, the produced SSO is complementary or substantially complementary to a SEQ ID NO as outlined in Table 1 and Table 3 and Tables 6-7, wherein the gene is selected from the group consisting of TXNRD1, DYRK1A, TRPM7 and PHLPP1; for use in the treatment of a diabetes. In a preferred embodiment, diabetes is selected from type 1 diabetes and type 2 diabetes.

In another embodiment, the produced SSO is complementary or substantially complementary to a SEQ ID NO as outlined in Table 1, Table 3 and Tables 6-7, wherein the gene is selected from the group consisting of LINGO2, SMAD2, ORC1, DDR2, STAG2, TRPM7, HIF1A, HTT, TAF2, CSPP1, RN115, LRRK2, UBAB2L, LRP6, MELK, and KNTC1. These 16 genes all comprise pseudoexons matching all criteria, all activated by SSO located within the Sweet Spot region (see Table 1 and Table 3 and Tables 6-7) and with high therapeutic potential.

In an embodiment, the method is computer-implemented. Thus, the invention can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

As mentioned above, the SSO may comprise one or more mismatches. An advantage of introducing such mismatches is that allele-specific targeting is possible. This may be relevant when you only want to target one allele of a gene.

Specific SSOs are listed in Table 4, 5 and 8 targeting the genes (pre-mRNA) of the listed genes. Thus, in an embodiment, the SSO is selected from the SSOs listed in Tables 4, 5 and 8.

### Composition for Use

The SSOs identified by the method of the invention, can be used as medicaments for the treatment of different diseases. Thus, another aspect of the invention relates to a composition comprising a splice switching oligonucleotide (SSO) being complementary or substantially complementary to a target pre-mRNA (e.g. encoding a functional disorder-causing or disorder-influencing protein), said target pre-mRNA (2) comprising:
a function-disabling pseudoexon comprising:
   at the 5′-end a 3′ splice site; and
   at the 3′-end a 5′ splice site;
      wherein said SSO is complementary or substantially complementary to the target pre-mRNA at a region +9 to +39 downstream to the 5′ splice site of said pseudoexon;
      wherein, when said SSO, in vivo, hybridizes to the pre-mRNA within the region +9 to +39 downstream to the 5′ splice site of said pseudoexon; said pseudoexon becomes part of the mature mRNA to a greater extent compared to corresponding pre-mRNA not contacted with the SSO;
      for use as a medicament.

Thus, the SSOs modulate expression of a target protein by promoting incorporation of a pseudoexon into the mature mRNA.

The modulation induced by the SSOs may influence the target protein in different ways. Thus, in an embodiment, hybridization of the SSO to the pre-mRNA results in:
decreasing the level of mRNA encoding the functional protein; and/or
decreasing expression of the functional protein; and/or
loss of function of the functional protein; and/or
a new function of the functional protein; and/or
mis-localization of the protein; and/or
mis-localization of the mRNA encoding the functional protein.

In an embodiment, the composition is for use in the treatment of a human subject having a disease or condition characterized by increased expression or altered function of the disorder-causing or disorder-influencing functional protein, or where decreased expression of the functional gene product is therapeutically beneficial.

In an embodiment, said SSO comprises a sequence which is complementary or substantially complementary to a polynucleotide in the pre-mRNA characterized by the parameters according to this invention.

In an embodiment, said SSO comprising a sequence which is substantially complementary to the polynucleotide in the pre-mRNA, comprises at the most 3 mismatches, such as at the most 2 mismatches or such as at the most 1 mismatch.

In an embodiment, said SSO comprises a sequence, which is complementary to a polynucleotide in the pre-mRNA according to the defined criteria.

In an embodiment, the region +9 to +39 relative to the 5′ splice site of said pseudoexon comprises a splicing regulatory site.

In yet an embodiment, the splicing regulatory site is an Intronic Splicing Silencer (ISS) site.

In an embodiment, said SSO has a length in the range 9-100 nucleotides, such as 9-50 nucleotides, preferably in the range 9-40 nucleotides and more preferably in the range 9-31 nucleotides or 9-25 nucleotides.

In an embodiment, said SSO comprises a sequence which is complementary or substantially complementary to a polynucleotide in the pre-mRNA as defined above, wherein said sequence has a length in the range 9-31 nucleotides, such as 9-20 nucleotides, preferably the sequence is complementary at a range of 9-31 nucleotides, such as 9-20, or such as 20-31 nucleotides, such as 25-31 nucleotides.

In an embodiment, said SSO comprises one or more artificial nucleotides, such as sugar-modified nucleotides.

In an embodiment, the oligonucleotide does not mediate RNAse H mediated degradation of the mRNA.

In an embodiment, at least one modified sugar moiety is a 2′-substituted sugar moiety.

In an embodiment, said 2′-substituted sugar moiety has a 2′-substitution selected from the group consisting of 2′-O-Methyl (2′-OMe), 2′-fluoro (2′-F), and 2′-O-methoxyethyl (2′-MOE).

In an embodiment, said 2′-substitution of said at least one 2′-substituted sugar moiety is a 2′-O-methoxyethyl (2′-MOE).

In an embodiment, the at least one modified sugar moiety is a bicyclic sugar moiety.

In an embodiment, the at least one bicyclic sugar moiety is a locked nucleic acid (LNA) or constrained ethyl (cEt) nucleoside.

In an embodiment, the at least one sugar moiety is a sugar surrogate.

In an embodiment, said at least one sugar surrogate is a morpholino.

In an embodiment, said at least one morpholino is a modified morpholino.

In an embodiment, the SSO comprises at least one internucleoside N3′ to P5′ phosphoramidate diester linkage.

In an embodiment, the modified oligonucleotide comprises at least one internucleoside phosphorothioate linkages.

In an embodiment, all internucleoside linkages are phosphorothioate.

In the example section, the tested SSOs were 25 nt long phosphorothioate RNA oligonucleotides with 2′-O-methyl modification on each sugar moiety.

In an embodiment, the SSO is conjugated to delivery elements, such as selected from the group consisting of Gal-Nac, (poly-)unsaturated fatty acids (such as oleoyl and linolenoyl), anisamide, anandamide, folic acid (FolA), carbachol, estrone, Retro-1, phospholipids, α-tocopherol (α-TP), cholesterol, squalene (SQ), unbranched fatty acids (such as lauroyl, myristoyl, palmitoyl, stearoyl, and docosanoyl), and cell penetrating peptides.

The composition can be used in the treatment of specific diseases. Thus in an embodiment, the composition for use in the treatment or alleviation of a disease selected from the group consisting of cancer, Inflammatory diseases, Neurodegenerative or neurological diseases, Metabolic conditions, Chronic liver disease and Inherited retinal dystrophies (IRDs).

In an embodiment,
the disease is cancer and the gene sequence is selected from the group consisting of ROCK1, TXNRD1, SLC7A11, STAT5B, MAPKAPK5, ZYG11A, MCCC2, SMYD2, DIAPH3, COPS3, SNX5, YBX1, CHD1L, PTPN11, UBAP2L, RNF115, HGS, TLK1, WWTR1, HMGCS1, SND1, THOC2, E2F3, LRIG2, HSPG2, SLC2A1, KNTC1, FDFT1, SMC1A, HIF1A, CSPP1, TRPM7, DDR2, STAG2, ORC1, TAF2, LRP6, MELK, TTBK2, TTK, ITGBL1, ROCK2, TASP1, FLT1, ZNF558, PMPCB, DBI, RAP1GDS, BUD1, CD44, CDKL5, ZNF558, RBPJ, EFEMP1, and FLT1.
or
the disease is an inflammatory disease and the gene sequence is selected from the group consisting of DDR2 TRPM7, SMAD2, and LRP6;
or
the disease is a neurodegenerative or neurological disease and the gene sequence is selected from the group consisting of ROCK1, OGA, TMEM97, PICALM, E2F3, SLC2A13, ASIC1, TRPM7, LRIG2, LRRK2, UBAP2L, SMC1A, TTBK2, ATXN7, CLCN1;
   or
   the disease is Chronic liver disease and the gene sequence is selected from the group consisting of SMAD2 and TRPM7, DDR2, HIF1A, and ROCK1, RAP1GDS1;
   or
   the disease is Diabetes and the gene sequence is selected from the group consisting of TXNRD1, DYRK1A, TRPM7 and PHLPP1.

Sweet Spot target sequences for some SSOs are provided in the example section (Table 1 and Table 3 and Table 6 and Table 7). Thus, in an embodiment, said composition for use comprises an SSO complementary or substantially complementary to region within a nucleic acid selected from the group consisting of
a nucleic acid according to any of any of SEQ ID NO's: 106, 1-26, 79-105, 107-125 and 137-201; or
a nucleic acid comprising 1 or 2 or 3 substitutions when compared to any of any of SEQ ID NO's: 106, 1-26, 79-105, 107-125 and 137-201; or
a nucleic acid sequence having at least 90% sequence identity to any of any of SEQ ID NO's: 106, 1-26, 79-105, 107-125 and 137-201;

In a preferred embodiment, said composition for use comprises an SSO complementary or substantially complementary to region within a nucleic acid selected from the group consisting of:
a nucleic acid according to any of SEQ ID NO's: 79-100; or
a nucleic acid comprising 1 or 2 or 3 substitutions when compared to any of SEQ ID NO's: 79-100; or
a nucleic acid sequence having at least 90% sequence identity to any of SEQ ID NO's: 79-100.

In a more preferred embodiment, said composition for use comprises an SSO complementary or substantially complementary to region within a nucleic acid selected from the group consisting of:
a nucleic acid according to any of SEQ ID NO's: 79-85; or
a nucleic acid comprising 1 or 2 or 3 substitutions when compared to any of SEQ ID NO's: 79-85; or
a nucleic acid sequence having at least 90% sequence identity to any of SEQ ID NO's: 79-85.

In an embodiment, the SSO is selected from the group consisting of:
a nucleic acid according to any of SEQ ID NO's: 127-136; or
a nucleic acid comprising 1 or 2 or 3 substitutions when compared to any of SEQ ID NO's: 127-136; or
a nucleic acid sequence having at least 90% sequence identity to any of SEQ ID NO's: 127-136.

In yet another preferred embodiment, the SSO is complementary or substantially complementary to region within a nucleic acid selected from the group consisting of
a nucleic acid according to any of SEQ ID NO: 100, 114, 118, 150 and 151;
a nucleic acid comprising 1 or 2 or 3 substitutions when compared to any of SEQ ID NO's: 100, 114, 118, 150 and 151; or
a nucleic acid sequence having at least 90% sequence identity to any of SEQ ID NO's: 100, 114, 118, 150 and 151.

In yet another preferred embodiment, the SSO is complementary or substantially complementary to region within a nucleic acid selected from the group consisting of
a nucleic acid according to any of SEQ ID NO: 12, 24 or 26;
a nucleic acid comprising 1 or 2 or 3 substitutions when compared to any of SEQ ID NO's: 12, 24 or 26; or
a nucleic acid sequence having at least 90% sequence identity to any of SEQ ID NO's: 12, 24 or 26.

Preferably, the SSO is SEQ ID NO: 128 or 136. As shown in examples 9 and 10, these SSOs have been optimized within the Sweet Spot region. Thus, by moving the binding region e.g. just 1 or 2 positions the efficiency can surprisingly be increased even further.

### Other Composition for Use

In yet an aspect, the invention relates to a composition for use as a medicament, said composition comprising
an SSO complementary or substantially complementary to region within a nucleic acid selected from the group consisting of
   a nucleic acid according to any of SEQ ID NO's: 79, or 1-26 or 80-125; or
   a nucleic acid comprising 1 or 2 or 3 substitutions when compared to any of SEQ ID NO's: 79, or 1-26 or 80-125; or
   a nucleic acid sequence having at least 90% sequence identity to any of SEQ ID NO's: 79, or 1-26 or 80-125;
      or
an SSO selected from the group consisting of:
   a nucleic acid according to any of SEQ ID NO's: 127-136; or
   a nucleic acid comprising 1 or 2 or 3 substitutions when compared to any of SEQ ID NO's: 127-136; or
   a nucleic acid sequence having at least 90% sequence identity to any of SEQ ID NO's: 127-136.

Preferably, the SSO is SEQ ID NO: 128 or 136. As shown in examples 9 and 10, these SSOs have been optimized within the Sweet Spot region. Thus, by moving the binding region e.g. just 1 or 2 positions the efficiency can surprisingly be increased even further.

As shown in examples 9 and 10, the SSOs targeting TRPM7 and HIF1A have been optimized within the Sweet Spot region.

Similar, optimization data for SMAD2 and LRRK2 are shown in Examples 3 and 13 respectively.

Thus, by moving the binding region just 1 or a few positions, the efficiency can surprisingly be increased even further.

As also described above and in the example section, different genes have already been targeted using the selection criteria according to the invention (Table 1 and Table 6) or has been identified as targeting sequences using the selection criteria according to the invention (Table 3 and Table 7). Thus, in an embodiment, the SSO is complementary or substantially complementary to region within a nucleic acid to a SEQ ID NO as outlined in Table 1, Table 3, Table 6 and Table 7 (Sweet Spot region).

In an embodiment, the SSO is complementary or substantially complementary to a SEQ ID NO as outlined in Table 1 and Table 3, wherein the gene is selected from the group consisting of TXNRD1, SLC7A11, STAT5B, MAPKAPK5, ZYG11A, ROCK1, MCCC2, SMYD2, DIAPH3, COPS3, SNX5, YBX1, CHD1L, PTPN11, UBAP2L, RNF115, HGS, TLK1, WWTR1, HMGCS1, SND1, HIF1A, CSPP1, TAF2, ORC1, THOC2, LRP6, MELK, TTBK2, TTK, ITGBL1, ROCK2, TASP1, FLT1, KNTC1, SMC1A, ZNF558, PMPCB and DBI; for use in the treatment of cancer.

In another embodiment, the SSO is complementary or substantially complementary to a SEQ ID NO as outlined in Table 1 and Table 3 and Table 6 and Table 7, wherein the gene is selected from the group consisting of ROCK1, E2F3, LRIG2, HSPG2, SLC2A1, KNTC1, DIAPH3, FDFT1, THOC2, SMC1A, DDR2, LINGO2, TRPM7, STAG2, RAP1GDS, BUD1, CD44, CDKL5, RNF115, UBAP2L, ZNF558, RBPJ, EFEMP1, FLT1; for use in the treatment of cancer.

In yet an embodiment, the SSO is complementary or substantially complementary to a SEQ ID NO as outlined in Table 1 and Table 3 and Table 6 and Table 7, wherein the gene is selected from the group consisting of ROCK1, OGA, TMEM97, PICALM, LRRK2, UBAP2L, SMC1A, and TTBK2; for use in the treatment of a neurological disease.

In an even more preferred embodiment, the neurological disease is selected from the group consisting of Alzheimer's disease, Amyotrophic lateral sclerosis, Friedreich's ataxia, Huntington's disease, Lewy body disease, Parkinson's disease and Spinal muscular atrophy.

Neurodegeneration is the progressive loss of structure or function of neurons, including death of neurons. Many neurodegenerative diseases—including amyotrophic lateral sclerosis, Parkinson's disease, Alzheimer's disease, Huntington's disease, and prion diseases—occur as a result of neurodegenerative processes.

In an embodiment, the SSO is complementary or substantially complementary to a SEQ ID NO as outlined in Table 1 and Table 3 and Table 6 and Table 7, wherein the gene is selected from the group consisting of ROCK1, E2F3, SLC2A13, LINGO2, TRPM7, ASIC1, LRIG2, LRRK2, UBAP2L, SMC1A, ATXN7, and CLCN1; for use in the treatment of a neurological disease.

Again, in an even more preferred embodiment, the neurological disease is selected from the group consisting of Alzheimer's disease, Amyotrophic lateral sclerosis, Friedreich's ataxia, Huntington's disease, Lewy body disease, Parkinson's disease and Spinal muscular atrophy.

In an embodiment, the SSO is complementary or substantially complementary to a SEQ ID NO as outlined in Table 1 and Table 3 and Table 6 and Table 7, wherein the gene is selected from the group consisting of TXNRD1, DYRK1A, TRPM7 and PHLPP1; for use in the treatment of a diabetes. In a preferred embodiment, diabetes is selected from type 1 diabetes and type 2 diabetes.

In another embodiment, the SSO is complementary or substantially complementary to a SEQ ID NO as outlined in Table 1 and Table 3 and Table 6 and Table 7, wherein the gene is selected from the group consisting of LINGO2, SMAD2, ORC1, DDR2, STAG2, TRPM7, HIF1A, HTT, TAF2, CSPP1, RN115, LRRK2, UBAB2L, LRP6, MELK, and KNTC1. These 16 genes all comprise pseudoexons matching all criteria, all activated by SSO located within the Sweet Spot region (see Table 1 and Table 3 and Table 6 and Table 7) and with high therapeutic potential.

### Compositions

In yet an aspect the invention relates to a
a composition comprising an SSO complementary or substantially complementary to region within a nucleic acid selected from the group consisting of
   a nucleic acid according to any of SEQ ID NO's: 106, 1-26, 79-105, 107-125 and 137-201; or
   a nucleic acid comprising 1 or 2 or 3 substitutions when compared to any of SEQ ID NO's: 106, 1-26, 79-105, 107-125 and 137-201; or
   a nucleic acid sequence having at least 90% sequence identity to any of SEQ ID NO's: 106, 1-26, 79-105, 107-125 and 137-201;
      or
a composition comprising an SSO selected from the group consisting of:
a nucleic acid according to any of SEQ ID NO's: 127-136 and SEQ ID NO's: 202-216; or
a nucleic acid comprising 1 or 2 or 3 substitutions when compared to any of SEQ ID NO's: 127-136 and SEQ ID NO's: 202-216; or
a nucleic acid sequence having at least 90% sequence identity to any of SEQ ID NO's: 127-136 and SEQ ID NO's: 202-216.

In an aspect the SSO is selected from the group of SSOs listed in table 4 (see example 9), Table 5 (see example 10) and Table 8 (see example 11).

Such compositions may be used as medicaments as outlined above, e.g. for the treatment of the list of diseases outlined above.

In another aspect the invention relates to a composition comprising a splice switching oligonucleotide (SSO) said composition comprising
an SSO complementary or substantially complementary to a region within a nucleic acid selected from the group consisting of
   a nucleic acid according to any of SEQ ID NO's: 217-294;
   a nucleic acid comprising 1 or 2 or 3 substitutions when compared to any of SEQ ID NO's: 217-294; or
   a nucleic acid sequence having at least 90% sequence identity to any of SEQ ID NO's: 217-294;
      or
an SSO selected from the group consisting of:
   a nucleic acid according to any of SEQ ID NO's: 295-367; or
   a nucleic acid comprising 1 or 2 or 3 substitutions when compared to any of SEQ ID NO's: 295-367; or
   a nucleic acid sequence having at least 90% sequence identity to any of SEQ ID NO's: 295-367;

### Allele Specific Targeting—SNP in Sweet Spot Region

By using the method according to the invention, the inventing team has identified SNPs inside a Sweet Spot region in the LRRK2 pre-mRNA (see also example 13).

By carefully designing SSOs it is considered plausible that such allele-specific SSOs can be used for preferentially targeting the disease-causing pre-mRNA (from the disease causing allele), whereas the pre-mRNA from the “normally functioning” allele is unaffected (or less affected).

By using such method, it is therefore possible to maintain an amount of normal RNA and thus maintaining normal gene-function.

As can been seen in example 13, SEQ ID NO's: 141 and 142 will target one SNP specific allele, whereas SEQ ID NO's: 158 and 159 will target another SNP specific allele.

Thus, in an embodiment the composition according to the invention is administered to a subject who is heterozygous in the pre-mRNA region targeted by the SSO, resulting in the SSO having an increased binding affinity to pre-mRNA of one of the alleles, such as to provide an increased splice switching activity in said allele.

In another embodiment, the pre-mRNA encodes for LRRK2.

In yet an embodiment, the pre-mRNA encodes for LRRK2 and the subject is heterozygous at the rs17444202 position.

In yet another embodiment, the pre-mRNA encodes for LRRK2 and the subject is heterozygous for a disease causing mutation in LRRK2.

In yet another embodiment, the SSO is complementary or substantially complementary to a region within a nucleic acid selected from the group consisting of
a nucleic acid according to any of SEQ ID NO's: 141, 142, 158 and 159; or
a nucleic acid comprising 1 or 2 or 3 substitutions when compared to any of SEQ ID NO's: 141, 142, 158 and 159; or
a nucleic acid sequence having at least 90% sequence identity to any of SEQ ID NO's: 141, 142, 158 and 159;
   or
the SSO is selected from the group consisting of:
a nucleic acid according to any of SEQ ID NO's: 208-216; or
a nucleic acid comprising 1 or 2 or 3 substitutions when compared to any of SEQ ID NO's: 208-216; or
a nucleic acid sequence having at least 90% sequence identity to any of SEQ ID NO's: 208-216;

In an embodiment, the SSO promotes inclusion of a pseudo-exon to a greater extent of disease-causing allele compared to the other allele. Again, this allows for the presence of the normal mRNA to a higher extent. Thus, the SSO has an increased binding affinity to pre-mRNA of a disease-causing allele.

Yet an aspect of the invention relates to a method for identifying a subject who is likely eligible for allele-specific targeting of a dysfunctional LRRK2 allele, the method comprising
determining in a DNA sample from said subject the allelic status for LRRK2;
   wherein if the subject is heterozygous for the rs17444202, said subject is eligible for allele-specific treatment according to the invention; or
   wherein if the subject is not heterozygous rs17444202, said subject is not eligible for allele-specific treatment according to the invention.

As also outlined further below, the subject may also be homozygous for the SNP, thus comprising the SNP on both alleles, which can promote pseudoexon inclusion. This may not be preferred, but it can be a better option if the alternative is no treatment.

Allelic status may be determined by Sanger or Next Generation (NGS) sequencing or by mutation specific assay, like ARMS or Taq-man.

### Allele Specific Targeting—SNP in Splice Sites

The inventing team has identified a wide range of SNPs which are present in the splice sites of a pseudoexon. When the SNP is present in one or both of the alleles, a subject harbouring such an SNP, may be eligible to allele specific SSO treatment (if the subject comprises the SNP only in the allele harbouring disease causing gene) or if the subject is homozygous for the SNP, thus harbouring the SNP on both alleles. Phrased in another way, the inventing team has identified SNPs, which increases the MaxEnt score of a splice site, such that pseudoexon inclusion becomes possible (or is improved), when treated with a corresponding SSO targeting said pseudoexon. See also examples 15-16.

Thus, an aspect of the invention relates to a composition comprising a splice switching oligonucleotide (SSO) for use as a medicament, said composition comprising
an SSO complementary or substantially complementary to a region within a nucleic acid selected from the group consisting of
   a nucleic acid according to any of SEQ ID NO's: 217-294;
   a nucleic acid comprising 1 or 2 or 3 substitutions when compared to any of SEQ ID NO's: 217-294; or
   a nucleic acid sequence having at least 90% sequence identity to any of SEQ ID NO's: 217-294;
      or
an SSO selected from the group consisting of:
   a nucleic acid according to any of SEQ ID NO's: 295-367; or
   a nucleic acid comprising 1 or 2 or 3 substitutions when compared to any of SEQ ID NO's: 295-367; or
   a nucleic acid sequence having at least 90% sequence identity to any of SEQ ID NO's: 295-367;
      wherein said SSO being complementary or substantially complementary to a target pre-mRNA, said target pre-mRNA comprising:
a function-disabling pseudoexon comprising:
   at the 5′-end a 3′ splice site; and
   at the 3′-end a 5′ splice site;
      wherein said SSO is complementary or substantially complementary to the target pre-RNA at a region +9 to +39 downstream to the 5′ splice site of said pseudoexon.

As illustrated in example 16, a wide range of SNPs has been identified which allow for SSO based pseudoexon inclusion treatment.

In an embodiment, the SSO is complementary or substantially complementary to a region within a nucleic acid selected from the group consisting of
a nucleic acid according to any of SEQ ID NO: 217;
a nucleic acid comprising 1 or 2 or 3 substitutions when compared to any of SEQ ID NO's: 217; or
a nucleic acid sequence having at least 90% sequence identity to any of SEQ ID NO's: 217.

Example 15 shows that when a specific SNP is present, an SSO targeting SEQ ID NO: 217 is able to direct inclusion of the pseudoexon into the mature mRNA.

In an embodiment, the composition is for use in the treatment of a human subject having a disease or condition characterized by increased expression or altered function of the disorder-causing or disorder-influencing functional protein, or where decreased expression of the functional gene product is therapeutically beneficial.

In another embodiment, said SSO comprises a sequence, which is substantially complementary to the polynucleotide in the pre-mRNA, comprises at the most 3 mismatches, such as at the most 2 mismatches or such as at the most 1 mismatch.

In a further embodiment, said SSO has a length in the range 9-100 nucleotides, such as 9-50 nucleotides, preferably in the range 9-40 nucleotides and more preferably in the range 9-31 nucleotides or 9-25 nucleotides.

In yet an embodiment, said SSO comprises a sequence which is complementary or substantially complementary to a polynucleotide in the pre-mRNA as defined above, wherein said sequence has a length in the range 9-31 nucleotides, such as 9-20 nucleotides, preferably the sequence is complementary at a range of 9-31 nucleotides, such as 9-20, or such as 20-31 nucleotides, such as 25-31 nucleotides.

In an embodiment, said SSO comprises one or more artificial nucleotides, such as sugar-modified nucleotides.

In an embodiment, the oligonucleotide does not mediate RNAse H mediated degradation of the mRNA.

In an embodiment, at least one modified sugar moiety is a 2′-substituted sugar moiety. In an embodiment, said 2′-substituted sugar moiety has a 2′-substitution selected from the group consisting of 2′-O-Methyl (2′-OMe), 2′-fluoro (2′-F), and 2′-O-methoxyethyl (2′-MOE).

In an embodiment, the composition is for use in the treatment or alleviation of a disease selected from the group consisting of cancer, inflammatory diseases, Neurodegenerative or neurological diseases, Metabolic conditions, Chronic liver disease and Inherited retinal dystrophies (IRDs).

As shown in example 15, the presence of the SNP allows for the SSO to promote inclusion of a function-disabling pseudoexon to a greater extent in a disease-causing allele compared to the other allele. Thus in an embodiment, the composition is administered to a subject who is heterozygous for a sequence variation (SNP) in the pre-mRNA targeted by the SSO, whereby the SSO promotes inclusion of a function-disabling pseudoexon to a greater extent in a disease-causing allele compared to the other allele.

The subject may also be homozygous. In such as case both the “healthy gene” and the diseased gene will be subject to pseudoexon inclusion. The subject may of course also harbour two diseased genes (one on each allele). Thus, in an embodiment, the composition is administered to a subject who is homozygous for the SNP variation (SNP) in the pre-mRNA targeted by the SSO.

In an embodiment, the subject is heterozygous for a sequence variation (SNP) in the 5′ splice site and/or the 3′ splice site, preferably the 5′ splice site of the function-disabling pseudoexon.

In an embodiment, the SNP is a point mutation, an insertion, such as of 1-20 nucleotides at the SNP position or a deletion, such as of 1-20 nucleotides at the SNP position.

In an embodiment, the insertion is 1-10 nucleotides, such as 1-5 nucleotides such as 2-4 nucleotides or 2-3 nucleotides. In another embodiment, the deletion is 1-nucleotides, such as 1-5 nucleotides such as 2-4 nucleotides or 2-3 nucleotides.

In another embodiment, the SNP is located in the (23-mer) 3′ splice site (−20 to +3 nt of the intron-exon border) or in the (9-mer) 5′ splice site (−3 to +6 nt of the exon-intron border).

In yet an embodiment, the heterozygosity in the disease-causing gene, increases inclusion of the pseudoexon into the mature mRNA to a larger extent from one allele than in the corresponding (normally functioning) gene on the other allele, when brought in contact with the SSO.

In another embodiment, the heterozygosity in the disease-causing gene increases the MaxEnt score of a splice site of a pseudoexon in the disease-causing gene compared to the corresponding (normally functioning) gene on the other allele. As outlined above the MaxEnt score is a defined number, calculated by a specific algorithm.

In a related embodiment, the heterozygosity in the disease-causing gene, results in a higher MaxEnt score of a splice site of a pseudoexon in the disease-causing gene compared to the corresponding (normally functioning) gene on the other allele.

In yet an embodiment, a splice site of the pseudoexon in the disease-causing gene allele, has a higher MaxEnt score compared to the corresponding splice site of the (normally functioning) gene on the other allele.

Databases uniquely identifying SNP with an ID exist. Thus, in an embodiment, the subject harbours a SNP in the disease-causing allele selected from the group of SNP IDs according to Table 9. Again, the SNP may also be present on both alleles. Thus, in an embodiment, the subject harbours a SNP on both alleles selected from the group of SNP IDs according to Table 9.

In a more specific embodiment, the subject harbours a SNP in the disease-causing allele selected from the group of SNPs according to Table 9 and the SSO targets a corresponding Sweet Spot Seq according to Table 9.

In an embodiment, the corresponding SSO is selected from Table 10.

In an embodiment, the pre-mRNA encodes for LRRK2. See also example 15 and 16.

In yet an embodiment, the pre-mRNA encodes for LRRK2 and the subject harbors the high MaxEnt score “G allele” of SNP rs10878372 in the disease causing LRRK2 allele.

In an embodiment, the pre-mRNA encodes for LRRK2 and the subject is heterozygous for a disease causing mutation in LRRK2.

It is to be understood that the targeted pseudoexon may be positioned in a gene, which is located on the X or the Y chromosome, in which case a male subject cannot be considered to be either heterozygous or homozygous, since they will only contain a single X and Y chromosome. However, such subjects may also be treated according to the invention.

As outlined throughout the application, the invention also relates to a composition comprising an SSO according to the invention, such as a pharmaceutical composition.

### Method for Identifying Subjects Eligible for Allele-Specific SSO-Based Pseudoexon Inclusion Treatment

The present invention may also be used for identifying subjects who are eligible for allele-specific SSO-based pseudoexon inclusion treatment. Thus, an aspect of the invention relates to a method for identifying a subject who is likely eligible for allele-specific SSO-based pseudoexon inclusion treatment of a dysfunctional or disease-causing gene, the method comprising
determining in a biological sample from said subject the presence of absence of heterozygosity in a pseudoexon of the dysfunctional or disease-causing gene;
   wherein if the subject is heterozygous for a sequence variation (SNP) in the 5′ splice site and/or the 3′ splice site, preferably the 5′ splice site of the function-disabling pseudoexon, said subject is likely eligible for allele-specific SSO-based pseudoexon inclusion treatment; or
   wherein if the subject is not heterozygous for a sequence variation (SNP) in the 5′ splice site and/or the 3′ splice site of the function-disabling pseudoexon, said subject is likely not eligible for allele-specific SSO-based pseudoexon inclusion treatment;
   wherein said SSO is complementary or substantially complementary to a target pre-mRNA of the dysfunctional or disease-causing gene, said target pre-mRNA comprising:
a function-disabling pseudoexon comprising:
   at the 5′-end a 3′ splice site; and
   at the 3′-end a 5′ splice site.

As outlined in examples 15 and 16, in certain cases the SSO will only work if the subject harbours an SNP, e.g. according to table 9.

In an embodiment, said allele-specific SSO-based pseudoexon inclusion treatment results in said SSO, in vivo, hybridizes to the pre-mRNA, and said pseudoexon of the dysfunctional or disease-causing gene becomes part of the mature mRNA to a greater extent compared to a corresponding pre-mRNA not contacted with the SSO.

In another embodiment, said allele-specific SSO-based pseudoexon inclusion treatment results in said SSO, in vivo, hybridizes to the pre-mRNA, and said pseudoexon becomes part of the mature mRNA to a greater extent in the allele harbouring the dysfunctional or disease-causing gene compared to corresponding pre-mRNA not contacted with the SSO.

In yet an embodiment, the heterozygosity for a sequence variation (SNP) in the 5′ splice site and/or the 3′ splice site of the pseudoexon in the pre-mRNA targeted by the SSO, will promote inclusion of the function-disabling pseudoexon to a greater extent of the disease-causing allele compared to the other allele, when/if brought in contact with the SSO.

In an embodiment, the heterozygosity for a sequence variation (SNP) is in the 5′ splice site and/or the 3′ splice site, preferably the 5′ splice site of the function-disabling pseudoexon.

In an embodiment, the heterozygosity for a sequence variation (SNP) in the disease-causing gene is a point mutation (SNP), an insertion of 1-20 nucleotides, or a deletion of 1-20 nucleotides, preferably a point mutation.

In an embodiment, the disease-causing gene increases inclusion of the pseudoexon to a larger extent than in the corresponding (normally functioning) gene on the other allele, when/if brought in contact with the SSO.

In an embodiment, the heterozygosity for a sequence variation (SNP) in the disease-causing gene increases the MaxEnt score of the splice site compared to the corresponding splice site in the (normally functioning) gene on the other allele.

In an embodiment, the heterozygosity for a sequence variation (SNP) in the disease-causing gene, results in a higher MaxEnt score of the splice site compared to the corresponding splice site in the (normally functioning) gene on the other allele.

In an embodiment, the heterozygosity for a sequence variation (SNP) in the disease-causing gene is in a splice site of the pseudoexon in the disease-causing gene, resulting in a higher MaxEnt score compared to the corresponding splice site of the (normally functioning) gene on the other allele.

In an embodiment, when said SSO, in vivo, hybridizes to the pre-mRNA, allele-specific SSO-based pseudoexon inclusion treatment includes treatment using an SSO which, in vivo, hybridizes to the pre-mRNA of the allele harbouring the disease-causing allele to a greater extent compared to the allele not harbouring the disease-causing allele.

In an embodiment said SSO is complementary or substantially complementary to the target pre-RNA at a region +9 to +39 downstream to the 5′ splice site of said pseudoexon.

In an embodiment, said target sequence for the SSO is positioned in a gene selected from the group consisting of LRRK2, LMN1B and ATXN2.

In an embodiment, the subject harbours a SNP in a disease-causing allele selected from the group of SNP IDs according to Table 9.

In an embodiment, the subject harbours a SNP in both alleles selected from the group of SNP IDs according to Table 9.

In an embodiment, the allelic status is determined by a method selected from the group consisting of such as Sanger sequencing, Next Generation sequencing (NGS) and by mutation specific assays, such as ARMS and Taq-man. The skilled person may find other methods suitable for determining the allelic status.

As also described above, a subject may be homozygous or heterozygous for a SNP (or the SNP may be X or Y chromosome associated). Thus, yet an aspect of the invention relates to a method for identifying a subject who is likely eligible for SSO-based pseudoexon inclusion treatment of a dysfunctional or disease-causing gene, the method comprising
determining in a biological sample from said subject the presence of absence of a SNP, which enables SSO-based pseudoexon inclusion treatment, in a pseudoexon of the dysfunctional or disease-causing gene;
   wherein if the subject has a sequence variation (SNP) in the 5′ splice site and/or the 3′ splice site, preferably the 5′ splice site of the function-disabling pseudoexon, said subject is likely eligible for SSO-based pseudoexon inclusion treatment; or
   wherein if the subject does not have a sequence variation (SNP) in the 5′ splice site and/or the 3′ splice site of the function-disabling pseudoexon, said subject is likely not eligible for SSO-based pseudoexon inclusion treatment;
wherein said SSO is complementary or substantially complementary to a target pre-mRNA of the dysfunctional or disease-causing gene, said target pre-mRNA comprising:
a function-disabling pseudoexon comprising:
at the 5′-end a 3′ splice site; and
at the 3′-end a 5′ splice site.

In an embodiment, the subject is either heterozygous for the SNP or homozygous for the SNP, or the SNP being X or Y chromosome associated, preferably heterozygous.

### Computer-Implemented

In an aspect, the invention relates to a computer program product being adapted to enable a computer system comprising at least one computer having data storage means in connection therewith to control a method according to the one or more aspects of the invention, such as a computer program product comprising instructions which, when the program is executed by a computer, cause the computer to carry out [the steps of] the method of the invention.

This aspect of the invention is particularly, but not exclusively, advantageous in that the present invention may be accomplished by a computer program product enabling a computer system to carry out the operations of the apparatus/system of the aspects of the invention when down- or uploaded into the computer system. Such a computer program product may be provided on any kind of computer readable medium, or through a network.

The individual aspects of the present invention may each be combined with any of the other aspects and embodiments. These and other aspects of the invention will be apparent from the following description with reference to the described embodiments.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

All patent and non-patent references cited in the present application, are hereby incorporated by reference in their entirety.

The invention will now be described in further details in the following non-limiting examples.

### EXAMPLES

### Example 1—Schematic Explanation of the Invention

FIG. 1 and the corresponding figure legend outlines the basic principle behind the invention. Pseudoexons are intronic sequences flanked by a 3′ and a 5′ splice site. Pseudoexons are usually not recognized due to the normally low amounts of inclusion into the mRNA transcript and because the pseudoexon containing mRNA is often degraded by the nonsense mediated decay of mRNA (NMD) system. The Sweet Spot region is defined as the region +9 to +39 nucleotides downstream of the 5′ splice site of a pseudoexon that obeys the criteria described (see e.g. example 2) in this application. Pseudoexon inclusion into the mRNA transcript can be activated and increased by employing SSOs complementary to the Sweet Spot region of pseudoexons fulfilling the criteria. Pseudoexon inclusion into the mRNA will modulate gene expression either at the mRNA level or protein level, by mislocalization, destabilization and degradation or alteration of protein function. 5′ss; 5′ splice site, 3′ss; 3′ splice site, SSO; splice shifting oligonucleotide.

FIG. 2 demonstrates how RNA-sequencing data can be used in detection of in vivo spliced double junctions for empirical detection of pseudoexons, which are included into the endogenous transcript at low levels.

After mapping to the human genome, reads are filtered to retain only fragments containing at least two splicing junctions. The splicing junctions of the entire fragment are then assembled into an exon structure, allowing for an unmapped gap between reads in the fragment of up to 100 bp. Exons are then classified using known exon annotations to identify pseudoexons contained within introns. Pseudoexons that may be candidates for activation by SSOs binding to the Sweet Spot region can be identified by the criteria according to the present invention, after which highly therapeutically relevant pseudoexons can be identified in genes where a downregulation of expression or alteration of the functional gene product is medically relevant. Subsequently, SSO can be produced using standard synthesis.

### Example 2—Identification of Selection Parameters for SSOs

### Aim of Study

Directly targeting specific genes as part of inhibiting a disease causing mRNA or protein in association with disease. By activating pseudoexons in target genes of interest, the resulting mRNA product will either be degraded through the NMD pathway or mis-localized or destabilized or being translated to a protein, which is non-functional or being unstable or mis-localized or having an altered function. We aimed to use Splice-switching antisense oligonucleotides (SSOs) to include pseudoexons in the mRNA transcript of the targeted gene. This strategy is superior to existing therapeutics that target multiple proteins, as the risk of off-target effects is minimized when using sequence specific SSOs, which are modified to achieve increased stability and binding specificity to their targeted sequence in a primary RNA transcript. Here we aimed to identify pseudoexons that can be activated, so that they are spliced into the mRNA by employing SSOs that bind in the +9 to +39 region (coined the Sweet Spot region) downstream of the pseudoexon donor site (5′-splice site), and to establish the criteria delineating these pseudoexons from non-activated pseudoexons.

### Materials and Methods

We used public RNA-sequencing data (Geuvadis, E-MTAB-2836, E-MTAB-513, GSE52946, and GSE124439) and mapped them with STAR after trimming for adapter contamination and poorquality bases with bbduk. HeLa cells were seeded in 12-well plates and forward transfected at 60% confluence with 20 or 40 nM 2′-O-methyl SSOs with full phosphorothioate backbone using Lipofectamine RNAiMAX (invitrogen). A non-binding ctrl SSO (5′GCUCAAUAUGCUACUGCCAUGCUUG3′) (SEQ ID NO: 126) was used as control. RNA was harvested after 48 hours using Trizol (Invitrogen) and chloroform to isolate the RNA, followed by precipitation with isopropanol. Complementary DNA (cDNA) was synthesized from 500 ng RNA using the High capacity cDNA kit (Applied Biosystems). Primers were designed to span at least one exon-exon junction of the neighboring exons flanking the pseudoexons of interest. PCR was carried out using TEMPase Hot Start DNA polymerase (ampliqon) and 1 μl cDNA per reaction. 0.5 pmol/μl of each primer was used. The PCR products were separated on a 2% Seakem LE (Lonza) TBE agarose gel, for 1 hour at 80V.

The Sweet Spot region is located +9 to +39 of the 5′ splice site of the pseudoexon.

All SSOs were 25 nt long phosphorothioate RNA oligonucleotides with 2′-O-methyl modification on each sugar moiety (Produced by LGC Biosearch Technologies).

SSOs were used targeting position+11 to +35 inside the Sweet Spot region for that gene (relative to the 5′ splice site of the pseudoexon). Thus, the SSOs binds inside the Sweet Spot.

### Results

In order to identify pseudoexons across multiple tissues, we collected public RNA-sequencing data representing many different cell types. After mapping to the human genome, we filtered all reads for fragments containing at least two splicing junctions. We then mapped the splicing junctions of the entire fragment, allowing for an unmapped gap between reads in the fragment of up to 100 bp. From this we compiled a non-degenerate list of fully spliced exons from which we extracted the unknown exons contained within introns. Using this double-junction approach, we were able to identify fully spliced pseudoexons even when expressed at very low levels. We have tested 78 SSOs targeting the Sweet Spot region from +11 to +35 downstream of randomly selected pseudoexons identified in RNA sequencing data. 26 of these SSOs were able to increase pseudoexon inclusion into the mRNA transcript of the targeted gene (Table 1 below), whereas the remaining 52 SSOs had no effect on pseudoexon inclusion (Table 2 below).

TABLE 1
26 pseudoexons matching all criteria, all activated by an SSO located
within the Sweet Spot region. Sweet Spot region is annotated
by its genomics equence (DNA).

| SEQ | | | |
|---|---|---|---|
| ID | | | |
| NO: | Gene | hg38 pseudoexon coordinates | Sweet Spot sequence |
| 1 | AGO3 | chr1: 36005718-36005858(+) | AAGTTTATAGGTTAAATATTTATTAAAGCCA |
| 2 | ARFGEF1 | chr8: 67226752-67226866(−) | ATTGCACAGCCTTCTTCAAGGAGGAGTCCCC |
| 3 | ATXN1 | chr6: 16432945-16433096(−) | TGAACTGTGTAGTGCCTCAGAAAATATCAAG |
| 4 | COPS5 | chr8: 67056866-67056932(−) | AATCCAGCCTACAGCCACCAAACTGGGTGGG |
| 5 | CSPP1 | chr8: 67162175-67162312(+) | ATGGCACATGTGGAGAATCTTCCATGGTGTG |
| 6 | CTNND2 | chr5: 11083880-11083955(−) | CAGCTCTGGGGTGCCTGCCCCCATGGGGGAA |
| 7 | DDR2 | chr1: 162756536-162756638(+) | CAGGAAGGGAGTGTGGAATTACAGCCTTCTA |
| 8 | DGCR8 | chr22: 20109208-20109297(+) | GTGCACCTGCCAGGGTTGAATCTGTAGGGCT |
| 9 | DMTF1 | chr7: 87184819-87184892(+) | CAGGCAGCGGCAGTGTAGCTTTTCCACTGTC |
| 10 | DMTF1 | chr7: 87167956-87168043(+) | ACATGAAAGCACATGAGCTGCTTAATGTTGG |
| 11 | EYA4 | chr6: 133301647-133301696(+) | CGGAACTGGGATTTGGACCTACCTTCAGAAC |
| 12 | HIF1A | chr14: 61724196-61724230(+) | AATTCAAGCTTAGTTTATGAAGGACTGAACA |
| 13 | HTT | chr4: 3144777-3144843(+) | GAGCTCAGCTCCTAAGGATGTGCAGGGGCAG |
| 14 | HTT | chr4: 3102605-3102748(+) | GGGGCACCAAAGTCTTCCCTGTCCCATCCCC |
| 15 | LINGO2 | chr9: 28370207-28370262(−) | GGATAGATAGAGAGGATCCAGAGGCTAAACG |
| 16 | NHSL1 | chr6: 138487729-138487783(−) | CAGGATATTGGAAGTCAGGTCCCATCTTCGT |
| 17 | NAA25 | chr12: 112049482-112049611(−) | TTGACTATTGGCCTATTTTCCTACTGGGCAA |
| 18 | ORC1 | chr1: 52394553-52394615(−) | CAAAAGCCATCTTTATGAGCACTCAGGTGCT |
| 19 | RBM22 | chr5: 150692025-150692147(−) | GTGAACTGACATTTGCTTAAGCCCCGTGTCA |
| 20 | SLC20A2 | chr8: 42471066-42471201(−) | CTGGTGTACTAAGAATACAGCCACATTCCCC |
| 21 | SMAD2 | chr18: 47868051-47868097(−) | GTCTTAGTTGTTGAAGCTAAGCGAGATGCAG |
| 22 | STAG2 | chrX: 124037905-124037969(+) | ACAGTGTAGGGAGTTAAAAAGTGCCATGAAT |
| 23 | TAF2 | chr8: 119779073-119779146(−) | ACGGAAAAGCGCCTTTCCCTCAGTTGTTCCT |
| 24 | TRPM7 | chr15: 50588190-50588250(−) | TCTTTAGTATATGCAGAAGTCAGAAAGTCAG |
| 25 | ZNF264 | chr19: 57206109-57206184(+) | AGCACCTACTCTCAGTCCACTACATTGAGTC |
| 26 | ZNF558 | chr19: 8814260-8814371(−) | TGCTCCCTGCAAGTACTAGGAATGCAGGCTG |

Underlining indicates tested or preferred binding sequence of the SSO.
Binding is to the corresponding pre-mRNA.
hg38 Genome coordinates follow BED format; zero-based start co-ordinate and one-based end coordinate.

TABLE 2
52 pseudoexons not matching all criteria, and not activated by SSO
located within the Sweet Spot region.

| SEQ | | | |
|---|---|---|---|
| ID | | | |
| NO: | Gene | hg38 pseudoexon coordinates | Sweet Spot sequence (5′ to 3′) |
| 27 | ARFGEF1 | chr8: 67260568-67260632(−) | TAAGTTTACCATCTTATATGGACGTGGTTTG |
| 28 | ATXN1 | chr6: 16442867-16443008(−) | TGGATTTTTGGTAGAGACGGGGTTTTGCCAT |
| 29 | ATXN1 | chr6: 16488491-16488637(−) | ACAGACCACTGAACCAAGAACCAAGAGAGAA |
| 30 | ATXN1 | chr6: 16319358-16319397(−) | CCTTTTCACATTGGCGTCTCTCACTTAGCGA |
| 31 | ATXN1 | chr6: 16494736-16494865(−) | CATCTATATGTTCAACTTACATTTTATTTTC |
| 32 | ATXN2 | chr12: 111520393-111520646(−) | CACTGTGCCTGGCCGGTTTTGTCTTCTAAGT |
| 33 | ATXN2 | chr12: 111554866-111555074(−) | TGGCTTCCTACCCCATTTATTTATACTTCAC |
| 34 | ATXN2 | chr12: 111461139-111461305(−) | TTCAGATCTTTTGAGCTAGAACAAAAAAACA |
| 35 | ATXN2 | chr12: 111529929-111530026(−) | TGTTAAATCTGATGTTAATGATTATTTCATC |
| 36 | ATXN2 | chr12: 111482787-111482831(−) | ATTATAGAGTTTAATCTTATTTTGAGGGCCT |
| 37 | ATXN2 | chr12: 111508911-111509038(−) | TCCTCAGCCTGTGTTTTAGCTTTCTAAATGT |
| 38 | CACNA1G | chr17: 50578805-50578849(+) | CAGCATGTGGGGAGAGGCGCGTCACTGGGAG |
| 39 | CACNA1H | chr16: 1217594-1217708(+) | GCAGGGGCCTCGGCCCAGGGGCTCCGACCTC |
| 40 | CEP128 | chr14: 80568955-80569044(−) | TTTCATAGGTAGGAATATTTAGGATATTTGG |
| 41 | CEP128 | chr14: 80527169-80527261(−) | CACTGTGTGTAGCCTTCTTCTTTCTCTATGG |
| 42 | CSPP1 | chr8: 67102087-67102144(+) | AGTTAGCTAATCATTTTCTCTGTTTATATTT |
| 43 | CTNND2 | chr5: 11119101-11119134(−) | GTAAGACTGACATGCCTTCTACCCTCCAGAA |
| 44 | ERAP2 | chr5: 96902643-96902842(+) | ACCCTCTAGTATAATATATGCCACATTAAAA |
| 45 | FKBP5 | chr6: 35671139-35671256(−) | CACCATGCCCAACTAACTTTTCTATTTTTTG |
| 46 | HTT | chr4: 3098129-3098222(+) | TACTGCTAAGTGGCATGTTTTGTTTTATGCT |
| 47 | HTT | chr4: 3234807-3234939(+) | GACTGGCCTGGGGTGTGGGAATCTAGGGCCT |
| 48 | HTT | chr4: 3124250-3124362(+) | CCCCCATTGAGAGCTGTGTCTTCAAACTCTT |
| 49 | HTT | chr4: 3213591-3213736(+) | GCTTTGTGGCAGAGAGGGGACTGGCACTTTG |
| 50 | HTT | chr4: 3218488-3218645(+) | TATTGATCAGAACCCTTGTTTCAGATAACAT |
| 51 | HTT | chr4: 3223670-3223869(+) | CATTTGGTATTACACCAGGTTCCTTTAGGCA |
| 52 | HTT | chr4: 3124250-3124305(+) | TCTTCTGGCTGGGACATGGGATATATCCTGT |
| 53 | LINGO2 | chr9: 28436349-28436392(−) | ACATATGTCTATGTTCAGTGCCAATAGTTAA |
| 54 | MAP2K2 | chr19: 4110359-4110415(−) | ATGTAGATGCCTTTGGTTTTGTTTTGTTTTC |
| 55 | MAP2K2 | chr19: 4109169-4109284(−) | CCGCCTGCCGCACAGCGTCGTTTGCAAAACC |
| 56 | MECP2 | chrX: 154094378-154094489(−) | AGATGTGAACAGGTCCCTCTTCTTTGGGCTT |
| 57 | MTOR | chr1: 11140517-11140666(−) | CCTGTTAGGAACCGGGAGGCACAGCAGGAGG |
| 58 | MUC16 | chr19: 8853333-8853363(−) | ATTTTTAGTTATTTGAGAAATCTCCACGTTT |
| 59 | NGLY1 | chr3: 25761678-25761708(−) | ATATTTATGTTTTTAAGAAATTACCAAGCTG |
| 60 | NUBPL | chr14: 31654070-31654134(+) | CATGAATTTTTTGGTTTCCCATTGCATATAA |
| 61 | NAA25 | chr12: 112045827-112045878(−) | TATTTTTAGTTACTTTTTATTTTTGAGACCT |
| 62 | PCSK9 | chr1: 55045413-55045449(+) | ACTCGCTGAAGTGGGGGCAGGTTAAGAAGCC |
| 63 | PCSK9 | chr1: 55055176-55055228(+) | GCAGCCAAGACTCTGTTCAAGTTTGTGTGGG |
| 64 | PSMG2 | chr18: 12705934-12706047(+) | TAAATAGTCTCAAAGGTGGAGGAGGCCCCAG |
| 65 | PTBP3 | chr9: 112300837-112300928(−) | CACTGTGCCCGGGTTTTTGTACCCCATATAA |
| 66 | SCN1A | chr2: 166007230-166007293(−) | TTACCCCTTTTGCTACCTTTAATCCTTGCAC |
| 67 | SCN1A | chr2: 165992896-165993078(−) | CAAGATCATGGGGAGATGAAAGTAGCATCAA |
| 68 | SCN9A | chr2: 166307149-166307277(−) | CTGATATTGATGTGAAAAATTGATATTTTGG |
| 69 | SCN9A | chr2: 166321439-166321515(−) | CCCACCTTAGCTACTCTCAAGCAGCTGAGAC |
| 70 | SDCCAG8 | chr1: 243497867-243497959(+) | CACTGTTGGGTTGTTTTTCTTTTGAAGTGTT |
| 71 | SNCA | chr4: 89771397-89771585(−) | AGGCATGCTGGACAAATGGATTCACATGTGC |
| 72 | SNCA | chr4: 89726926-89726976(−) | TCATTAAATGGTGCATCCGGATCAGAACCTA |
| 73 | SNCA | chr4: 89836461-89836529(−) | CCAACTTTTCTCTCACATAAAATCTGTCTGC |
| 74 | SNCA | chr4: 89836337-89836394(−) | GGGTTAACAAGTGCTGGCGCGGGGTCCGCTA |
| 75 | SORT1 | chr1: 109397207-109397340(−) | TCTCTGGGTCAGTACTTTCCGGGTGGGAGAG |
| 76 | SP100 | chr2: 230436381-230436405(+) | ATCATGGGGCAATTTCACCCATTCTGTTCTC |
| 77 | TMEM243 | chr7: 87206679-87206742(−) | TAAACTGTGCACTGTTCTATGTAATATGATT |
| 78 | YIPF6 | chrX: 68506157-68506258(+) | GATCCCCATATACACCTTCAACAATTATCTA |

Underlining indicates tested or preferred binding sequence of the SSO.
Binding is to the corresponding pre-mRNA.
hg38 Genome coordinates follow GFF/GTF format; one-based start and end coordinates.

Based on these results we established a set of criteria, which must be met in order for a pseudoexon to be included by an SSO targeting the Sweet Spot region. Using these criteria, we have selected new targets for pseudoexon inclusion in disease causing genes where pseudoexon inclusion has a high potential for therapeutic use in human diseases.

### Criteria:

### For the Pseudoexon:

Pseudoexon length<160 nt;
Pseudoexon length>30 nt;
The last 3 nt of the pseudoexon are different from TAG;
Donor splice site has a MaxEnt score≥4.33;
Donor splice site has a MaxEnt score≤10.06; and
Acceptor splice site has a MaxEnt score≥3.63;

### For the Gene Sequences +9 to +39 Downstream to the 5′ Splice Site of Said Pseudoexon (3):

Total pyrimidines≤20;
Total thymidine bases≤12;
Total thymidine bases≥4;
Total guanine bases≤12;
Maximum length of thymidine polymer≤4;
Maximum length of pyrimidine polymer≤10;
Minimum length of purine polymer≥3; and
Maximum number of guanine polymers of at least 3 nt length≤2;

### Conclusion

Pseudoexons in target genes of interest can be activated as a mechanism for downregulation of a disease-causing protein. By filtering pseudoexons based on the criteria we have established, the selection of new target candidates will enable the discovery of novel therapeutic agents.

### Example 3—SMAD2

### Aim of Study

Chronic liver disease is characterized by inflammation and fibrosis of the liver. Through in silico analysis and in vivo experiments, we aimed at investigating the presence of pseudoexons in the TGF-β/Smad signaling pathway, which is important for tissue fibrosis (Inagaki et al, 2007).

### Materials and Methods

We used our novel double-junction approach to identify fragments with fully spliced pseudoexons in publicly available RNA-seq data from the GEUVADIS consortium. HeLa cells, LX-2 and HepG2 cells were seeded in 12-well plates and forward transfected at 60% confluence with 40 nM SSO using Lipofectamine RNAiMAX (invitrogen). SSO (targeting SEQ ID NO: 21; specific target sequence is underlined in SEQ ID NO: 21 in table 1) was 25 nt long phosphorothioate RNA oligonucleotides with 2′-O-methyl modification on each sugar moiety (Produced by LGC Biosearch Technologies). (See also table from example 2). The SSO is complementary to position +11 to +35 inside the Sweet Spot region for the SMAD2 gene (relative to the 5′ splice site of the pseudoexon) (SEQ ID NO: 21). Thus, the SSOs binds inside the Sweet Spot. In order to determine the optimal SSOs targeting the Sweet Spot region, we tested several SSOs employing 25 nt long SSOs targeting the Sweet Spot region from +9 to +14 position downstream of the 5′ss of the PE (SEQ ID NO: 202-207 listed in table 8 in example 11).

A non-binding SSO (5′-GCUCAAUAUGCUACUGCCAUGCUUG-3′) (SEQ ID NO: 126) with similar modifications was used as a negative control. For experiments with TGFβ stimulation, the cells were stimulated with 10 ng/ml TGFβ (R&D systems) for 16 hours before RNA and protein harvest. RNA was harvested after 48 hours using Trizol (Invitrogen) and chloroform to isolate the RNA, followed by precipitation with isopropanol. Complementary DNA (cDNA) was synthesized from 500 ng RNA using the High capacity cDNA kit (Applied Biosystems). Primers were designed to span at least one exon-exon junction of the neighboring exons flanking the pseudoexons of interest. PCR was carried out using TEMPase Hot Start DNA polymerase (ampliqon) and 1 μl cDNA per reaction. 0.5 pmol/μl of each primer was used. The PCR products were separated on a 2% Seakem LE (Lonza) TBE agarose gel, for 1 hour at 80V. Proteins were extracted by lysing the cells with okaidic acid to preserve phosphorylated proteins, benzonase treated, and the denatured proteins were separated on a 4-12% NuPage SDS-Page gel and analyzed by western blotting using antibodies against SMAD2, phosphoSMAD2 and actin for control. For the study of myofibroblast formation, LX-2 cells were grown in 96 well plates transfected with 20 nM SSO and incubated in the Incucyte instrument with images takes every 4 hours. The images were analyzed in ImageJ by making a mask for spherical (differentiated) cells.

### Results

By examining fragments with fully spliced pseudoexons, we identified a pseudoexon located within intron 5 of the SMAD2 gene, encoding the signal transducer protein Mothers against decapentaplegic homolog 2, which is involved in the TGF-β/Smad pathway. Inclusion of the pseudoexon into a mature mRNA results in the insertion of 46 nt into the coding region, causing a frame-shift. A stop-codon (UAA) is also located within the pseudoexon, potentially activating the NMD pathway, causing degradation of the transcript.

Transfection of SSOs (SEQ ID NO: 202-207 listed in table 8, example 11) showed that all mediate pseudoexon inclusion, and that SSO targeting from +11 and +12 are optimal in mediating pseudoexon inclusion into the SMAD2 transcript (results not shown).

Transfection of HeLa cells with an SSO complementary to position +11 to +35 downstream of the 5′ splice site resulted in up to 90% inclusion of the pseudoexon (FIG. 8A). Transfection of the same SSO in LX-2 Hepatic stellate cells resulted in even higher pseudoexon inclusion with lower concentration of SSO (FIG. 8B). Protein levels from HepG2 cells show a high decrease in SMAD2 protein, as well as phosphorylated SMAD2 under TGFβ stimulation (FIG. 8C). TGFβ stimulation of LX-2 cells resulted in differentiation of the cells into myofibroblast, which was reduced by transfection with the +11 SSO (FIG. 8D).

### Conclusion

The normal function of the SMAD2 gene may be decreased by up to 90% using a specific SSO to increase inclusion of a pseudoexon thereby disrupting the function of the normal gene product, either through degradation of the transcript or expression of a truncated and non-functional protein. This has relevance in hepatic fibrosis and other disorders associated with increased TGF-β activity, of which SMAD2 is a positive regulator (Sysa et al, 2009). Additionally, SMAD2 downregulation may reduce growth of gliomas (Papachristodoulou et al, 2019)
Inagaki Y, et al. Gut. 2007 February; 56(2):284-92. doi: 10.1136/gut.2005.088690 Sysa P, et al. 2009 September; 28(9):425-34. doi: 10.1089/dna.2009.0884Papachristodoulou A, et al. Clin Cancer Res. 2019 Dec. 1; 25(23):7189-7201. doi: 10.1158/1078-0432.CCR-17-3024

### Example 4—ORC1

### Aim of Study

The origin of recognition complex (ORC) genes are involved in DNA replication and are expressed highly in hepatocellular carcinoma tumors (Wang et al, 2020). Low expression of ORC1 consistently indicates better prognosis compared to high ORC1 expression (Wang et al, 2020), while knockdown of ORC1 sensitises cancer cells, making them vulnerable to other anticancer treatments (Zimmerman et al, 2013). Through in silico analysis and in vivo experiments, we aimed at investigating the presence of pseudoexons in the ORC1 gene, which may be used to down-regulate the expression this gene.

### Materials and Methods

We used our novel double-junction approach to identify fragments with fully spliced pseudoexons in publicly available RNA-seq data from the GEUVADIS consortium. HeLa cells were seeded in 12-well plates and forward transfected at 60% confluence with 40 nM SSO using Lipofectamine RNAiMAX (invitrogen).

SSO (targeting SEQ ID NO: 18; specific target sequence is underlined in SEQ ID NO: 18 in table 1) was 25 nt long phosphorothioate RNA oligonucleotides with 2′-O-methyl modification on each sugar moiety (Produced by LGC Biosearch Technologies). (See also table from example 2). The SSO is complementary to position +11 to +35 inside the Sweet Spot region for the ORC1 gene (relative to the 5′ splice site of the pseudoexon). Thus, the SSOs binds inside the Sweet Spot.

A non-binding SSO (5′GCUCAAUAUGCUACUGCCAUGCUUG3′) (SEQ ID NO: 126) with similar modifications was used as a negative control. RNA was harvested after 48 hours using Trizol (Invitrogen) and chloroform to isolate the RNA, followed by precipitation with isopropanol. Complementary DNA (cDNA) was synthesized from 500 ng RNA using the High capacity cDNA kit (Applied Biosystems). Primers were designed to span at least one exon-exon junction of the neighboring exons flanking the pseudoexons of interest. PCR was carried out using TEMPase Hot Start DNA polymerase (ampliqon) and 1 μl cDNA per reaction. 0.5 pmol/μl of each primer was used. The PCR products were separated on a 2% Seakem LE (Lonza) TBE agarose gel, for 1 hour at 80V.

### Results

Using our double-junction approach to examine fragments with fully spliced pseudoexons, we identified a pseudoexon within intron 5 of the ORC1 gene, resulting in insertion of 62 nt into the pre-mRNA causing a frame-shift. Transfection of HeLa cells with an SSO complementary to position +11 to +35 downstream of the 5′ splice site resulted in up to 45% more inclusion of the pseudoexon (data not shown).

### Conclusion

The normal expression of the ORC1 gene product may be decreased by up to 45% using a specific SSO to increase inclusion of a pseudoexon disrupting function of the normal gene product. SSO-mediated down-regulation of ORC1 expression might therefore work in enhancing the anti-cancer effect of other drugs.
Wang X K, et al. J Cancer. 2020 Jan. 20; 11(7):1869-1882. doi: 10.7150/jca.39163 Zimmerman, K. M., et al. Mol Cancer Res, 2013. 11(4): p. 370-80.

### Example 5—LINGO2

### Aim of Study

LINGO2 (Leucine-rich repeat and immunoglobulin-like domain-containing nogo receptor-interacting protein 2) is highly expressed in many tissues including intestinal tissues, brain and neurons (such as cortical neurons and dorsal root ganglion (DRG) neurons) (Guillemain et al. 2020). LINGO2 expression is increased in gastric cancer and this is associated with a poor prognosis and down regulation of LINGO2 decreases proliferation of gastric cancer cells (Jo et al. 2019). LINGO2 also negatively regulates motor neuron survival and motor neuron axonal length. This suggests that knock down or alteration of LINGO2 expression could be a new therapy against neuronal disorders and cancer.

### Materials and Methods

We used the analysis pipeline (FIG. 2) to analyze publicly available RNA-seq data. HeLa cells were grown in RPMI and transfected with 20 nM SSO using Lipofectamine. After 24 hours, cells were harvested and RNA purified. RT-PCR was performed and the resulting product visualized on 2% agarose gel. For Incucyte experiments U251 cells were grown in 96-well plates transfected with SSOs at 5, 10, 20 or 40 nM and incubated in the incucyte instrument with images taken every 4^{th} hour.

### Results

Based on the high LINGO2 expression in neurons and brain, we speculated if SSO based activation of the PE could be used for treating glioblastoma. By employing an SSO targeting the Sweet Spot region according to SEQ ID NO: 15 (specific target sequence is underlined in SEQ ID NO: 15 in table 1) we demonstrated a high level of inclusion of the chr9:28370208-28370262 (−) LINGO2 pseudoexon in U251 glioblastoma cells (FIG. 3A). This pseudoexon introduces 55 bp between exon 3 and 4 in the mRNA of the pseudoexon. When investigating U251 glioblastoma cells with this SSO, we observed a dose dependent reduction of growth and proliferation of U251 glioblastoma cells resulting from SSO treatment (FIG. 3B).

### Conclusion

Accordingly, methods for treating cancer, such as gastric cancer or glioblastoma or for promoting survival of motor neurons and axonal growth of motor neurons by contacting human cells, such as cancer or neuronal cells with an SSO that causes inclusion of the pseudoexon into LINGO2 mRNA are provided herein.

### Example 6—TAF2

### Aim of Study

The TAF2 gene expresses the Tata-box binding protein associated factor 2, a subunit of the transcription factor II D complex involved in binding to promotor sequences to initiate transcription (Martinez et al. 1998). TAF2 exhibits copy number increases or mRNA overexpression in 73% of high-grade serous ovarian cancers (HGSC) (Ribeiro et al. 2014) and is important in cancer.

### Materials and Methods

We used the analysis pipeline (FIG. 2) to analyze publicly available RNA-seq data. NCI-H358 lung cancer cells cells were grown in RPMI and transfected with nM SSO using Lipofectamine. After 24 hours, cells were harvested and RNA purified. RT-PCR was performed and the resulting product visualized on 2% agarose gel. WST-1 assay of was performed 48 hours post transfection. For Incucyte experiments NCI-H358 lung cancer cells were grown in 96-well plates transfected with SSOs at 10 nM and incubated in the incucyte instrument with images taken every 4^{th} hour.

### Results

Inclusion of the chr8:119779073-119779146(−) TAF2 pseudoexon will introduce 73 bp between exon 17 and 18 in the mRNA, resulting in a shifted reading frame with a premature stop codon in exon 18. TAF2 mRNA transcripts with inclusion of this pseudoexon are targets for nonsense-mediated mRNA decay (NMD), and increased pseudoexon inclusion induced by SSO treatment will therefore result in a reduction of expression of TAF2 mRNA. Moreover, if translated the pseudoexon included transcript will result in production of a severely truncated protein without normal TAF2 function.

By employing an SSO targeting the TAF2 Sweet Spot region according to SEQ ID NO: 23 (specific target sequence is underlined in SEQ ID NO: 23 in table 1) we demonstrated a high level of inclusion of chr8:119779073-119779146(−) TAF2 pseudoexon in NCI-H358 lung cancer cells (FIG. 4A). When investigating NCI-H358 lung cancer cells with this SSO, we observed reduction of growth and proliferation of by incucyte assay (FIG. 4B) and by WST-1 assay (FIG. 4C).

### Conclusion

Employing the SSO targeting the TAF2 pseudoexon results in increased pseudoexon inclusion and reduced proliferation of cancer cells. SSO targeting TAF2 pseudoexon might therefore be candidates to be used in future anti-cancer therapy.

### Example 7—HTT

### Aim of Study

HTT encodes the huntingtin protein and is associated with the autosomal dominant neurodegenerative disorder, Huntington's disease, which is caused by unstable expansion of CAG trinucleotide repeats in the HTT gene that results in translation of a cytotoxic mutant protein with an abnormal polyglutamine tract. Downregulation of HTT has been studied as a potential strategy in treatment of Huntington's disease by reducing levels of mutant huntingtin. Inclusion of the chr4:3102605-3102748(+) HHT pseudoexon will introduce 143 bp between exon 3 and 4 in the mRNA, including an in-frame premature termination codon. HTT mRNA transcripts with inclusion of this pseudoexon is a target for nonsense-mediated decay (NMD), and increased pseudoexon inclusion induced by SSO treatment will result in a reduction of expression of HTT mRNA. Huntington disease is an autosomal dominant disease caused by a dominant negative tri-nucleotide repeat expansion in HTT mRNA. Inclusion of the PE will reduce levels of the dominant negative mRNA and may therefore be used as treatment.

### Materials and Methods

We used the analysis pipeline (FIG. 2) to analyze publicly available RNA-seq data. HeLa cells were grown in RPMI and transfected with 20 nM SSO using Lipofectamine. After 24 hours, cells were harvested and RNA purified. RT-PCR was performed and the resulting product visualized on 2% agarose gel.

### Results

By employing an SSO targeting the Sweet Spot region according to SEQ ID NO: 14 (specific target sequence is underlined in SEQ ID NO: 14 in table 1) we demonstrated a high level of inclusion of the chr4:3102605-3102748(+) HHT pseudoexon in HeLa cells which causes degradation of HTT mRNA by the NMD system (Data not shown).

### Conclusion

Employing the SSO targeting the HTT pseudoexon results in increased pseudoexon inclusion, which is suited to downregulate dominant negative mRNA that causes Huntington Disease.

### Example 8—Further Relevant Targets

The following additional Sweet Spot sequences were identified using the criteria according to the invention (see e.g. example 2). Thus, these targets will with very high plausibility be functional targets for SSOs, allowing for incorporation of the pseudoexon in the mature mRNA.

TABLE 3
Relevant target genes

| SEQ | | | |
|---|---|---|---|
| ID | | | |
| NO: | Gene | hg38 pseudoexon coordinates | Sweet Spot sequence |
| 79 | ROCK1 | chr18: 21017021-21017098(−) | CTGTGAAGGCCGTGAGGTAAGAGACCTTGAC |
| 80 | ROCK1 | chr18: 21022445-21022564(−) | GGAATGGGGGAAATGGAGAATAACCAGTATA |
| 81 | OGA | chr10: 101795365-101795480(−) | AAGGGCGGTAGACATGAACTGAAGTCACGTT |
| 82 | OGA | chr10: 101795374-101795480(−) | GGTAGATTTAAGGGCGGTAGACATGAACTGA |
| 83 | TMEM97 | chr17: 28320422-28320470(+) | CGTGTAGCAAGTTGCAGTCTGGGGACTTGGT |
| 84 | TXNRD1 | chr12: 104300119-104300276(+) | TGAACCCTGGGTAGCAACTCTTGAGCGAAGA |
| 85 | SLC7A11 | chr4: 138191360-138191415(−) | TAGATTAGACATCAGTGGTATTGAAATTTAA |
| 86 | STAT5B | chr17: 42229494-42229533(−) | TGCTCAGAGACTGGATCCTTTTAAGAGTGGG |
| 87 | MAPKAPK5 | chr12: 111870683-111870782(+) | CTTATAGAGTGAAGGGTCCCTAGGCCAAGAC |
| 88 | ZYG11A | chr1: 52880598-52880653(+) | TTGGAGGAGGGAGAAGCCCACCTTTTAAGGA |
| 89 | ZYG11A | chr1: 52880601-52880653(+) | TTGGAGGAGGGAGAAGCCCACCTTTTAAGGA |
| 90 | MCCC2 | chr5: 71642612-71642727(+) | CAGTGCCATTTAGGATGTACTGCATAAGTTT |
| 91 | MCCC2 | chr5: 71642664-71642727(+) | CAGTGCCATTTAGGATGTACTGCATAAGTTT |
| 92 | SMYD2 | chr1: 214334511-214334649(+) | TGGAGAGTTAGGGTTAGGATGTTAGAATTGC |
| 93 | DIAPH3 | chr13: 59923001-59923041(−) | TAGCATTAACAGTATAGGTAGGGAATCTGGT |
| 94 | DIAPH3 | chr13: 59923001-59923111(−) | TAGCATTAACAGTATAGGTAGGGAATCTGGT |
| 95 | COPS3 | chr17: 17260072-17260112(−) | CAAGGCTACATTGGGAGACAGTGAAAGGCAG |
| 96 | SNX5 | chr20: 17948072-17948142(−) | CCAGAAGCAGGATTTGCAGGGTAGGGTTATG |
| 97 | YBX1 | chr1: 42699646-42699724(+) | CACTGTGCCCAACCTTGGAGTGAGATGAATA |
| 98 | CHD1L | chr1: 147276877-147276929(+) | GGAGTAATAAATGTCTGTCAAGGGCAGCATC |
| 99 | PTPN11 | chr12: 112456115-112456268(+) | TCAATGGATGTGCTAGCCGCTCCATTTGGCT |
| 100 | UBAP2L | chr1: 154241700-154241797(+) | AATATCTGATTGTCGGATTTATCCCAGGAAG |
| 101 | E2F3 | chr6: 20456984-20457037(+) | GGCTTCAGAGATGAAGAGAGTATTTCTCCTC |
| 102 | SLC2A13 | chr12: 39831850-39831885(−) | ATCAGGAAGCTTATGGTAGAAGGCTAAGGGG |
| 103 | SLC2A13 | chr12: 39939133-39939264(−) | GCTATGTCAGTAGGCAACTGGAGCACAGTCC |
| 104 | ASIC1 | chr12: 50079657-50079740(+) | TTGGCAGAGTTTAGCATCCAGGCAGGGTGAA |
| 105 | LRIG2 | chr1: 113116745-113116798(+) | TCAGTAAAGTTTAAAGGGTAGCTAGGAGCCA |
| 106 | RNF115 | chr1: 145784178-145784251(−) | CAGGCCAGGATAAGTTGTCGAAGTCACAATG |
| 107 | HGS | chr17: 81684813-81684891(+) | GTGCTTCAGGGATGAATCCAGAGGTTAACTA |
| 108 | TLK1 | chr2: 171026752-171026849(−) | CTGTGTGCCCACTTAAACTGTTTTGATAATG |
| 109 | WWTR1 | chr3: 149571657-149571724(−) | GAACTCAAGTAGACCCTAGTCAGCTTCAAGT |
| 110 | HMGCS1 | chr5: 43297529-43297673(−) | TGTCATCCCCACTTTACAGAGAAGTGGGTTA |
| 111 | SND1 | chr7: 127725671-127725726(+) | GCCTTTTCCAAACCAAAGAACAAAGCGGGAG |
| 112 | HSPG2 | chr1: 21898326-21898469(−) | TTTAGGCCAAAGCAGGAATAAGATGTGGACA |
| 113 | SLC2A1 | chr1: 42942082-42942153(−) | AGTGACTCCCAAAGATCAGGGTCCTGTTTGG |
| 114 | KNTC1 | chr12: 122595478-122595600(+) | AGGACTATAGGCTGCGCATAGCTCGAAGGAG |
| 115 | FDFT1 | chr8: 11826877-11826978(+) | AAACTCCAGGTGTCTGTAATAAGGGACAGGG |
| 116 | THOC2 | chrX: 123683845-123683911(−) | CCTGAGGTTGGGAGTTTGAGAGCAGCCTGAA |
| 117 | THOC2 | chrX: 123706588-123706704(−) | GTAGGGAGAAAAAAAAAAGCCTGTGCTGTAA |
| 118 | SMC1A | chrX: 53402287-53402379(−) | GATGAACACAACACAGTCTTTGCTGAAGGAG |
| 119 | PICALM | chr11: 85978968-85979061(−) | GGAGTTTAGAGTAGTGGATTTTATGGACTCG |
| 120 | PICALM | chr11: 85984424-85984547(−) | GAGGGAGAAAAACTTTTATCTTGGGGCTACT |
| 121 | DYRK1A | chr21: 37383084-37383160(+) | AATCTGTATTCTCTAGTCAAGGAGGGATGGG |
| 122 | PHLPP1 | chr18: 62885960-62886035(+) | ACTAGTAGATCACACCCCTAGATAGTTTAGC |
| 123 | AURKA | chr20: 56390967-56391068(−) | AATTGGAGCAAATGCCTGTAGCTTCTGTCAG |
| 124 | BACH1 | chr21: 29372435-29372563(+) | TTACAGTCCGTTGTAAATGAGTGACCTAGCG |
| 125 | MAP4 | chr3: 47870692-47870737(−) | GCCGGGGCTCTGTAGTGGGTTTTGTGCCCCA |

Underlinings indicate tested or preferred binding sequence of the SSO.
Binding is to the corresponding pre-mRNA.
hg38 Genome coordinates follow GFF/GTF format; one-based start and end coordinates.

### Further Explanation of Target Genes:

### ROCK1:

ROCK1 encodes a Rho associated serine/threonine kinase. The signaling pathway of ROCK1 has been associated with the pathogenesis of metabolic diseases and several neurodegenerative disorders, like Huntington's disease, Parkinson's disease, and Alzheimer's disease, and is a promising target for treatment of neurodegenerative disorders by suppression of its function Koch et al. 2018). Inhibition of ROCK1 is a potent target for treatment of chronic ophthalmological diseases (Moshifar et al. 2018). Hepatic ROCK1 is a suggested target for treatment of nonalcoholic fatty liver disease and hepatocellular carcinoma (Huang et al. 2018; Wu et al 2021).

Inclusion of the chr18:21022445-21022564(−) ROCK1 pseudoexon will introduce 120 bp between exon 11 and 12 in the mRNA, including an in-frame premature termination codon. mRNA transcript with inclusion of this pseudoexon is predicted as a target of nonsense-mediated decay, and increased pseudoexon inclusion will result in a reduction of gene expression.

Inclusion of the chr18:21017021-21017098(−) ROCK1 pseudoexon will introduce 78 bp between exon 12 and 13 in the mRNA. In translation of the protein, this will introduce 26 amino acids to the amino acid sequence. This will potentially reduce gene expression by disruption of protein function or alter normal protein function.

Sweet Spots for ROCK1 SSO targeting are shown in Table 3 and Table 6. Koch J C, Tatenhorst L, Roser A E, Saal K A, Tönges L, Lingor P. ROCK inhibition in models of neurodegeneration and its potential for clinical translation. Pharmacol Ther. 2018 September; 189:1-21. Moshirfar M, Parker L, Birdsong O C, Ronquillo Y C, Hofstedt D, Shah T J, Gomez A T, Hoopes PCS. Use of Rho kinase Inhibitors in Ophthalmology: A Review of the Literature. Med Hypothesis Discov Innov Ophthalmol. 2018 Fall; 7(3):101-111. Huang H, Lee S H, Sousa-Lima I, Kim S S, Hwang W M, Dagon Y, Yang W M, Cho S, Kang M C, Seo J A, Shibata M, Cho H, Belew G D, Bhin J, Desai B N, Ryu M J, Shong M, Li P, Meng H, Chung B H, Hwang D, Kim M S, Park K S, Macedo M P, White M, Jones J, Kim Y B. Rho-kinase/AMPK axis regulates hepatic lipogenesis during overnutrition. J Clin Invest. 2018 Dec. 3; 128(12):5335-5350. Wu H et al. (2021) Biochemical PharmacoloqyVolume 184, February 2021, 114353. (https://doi.orq/10.1016/j.bcp.2020.114353)

### OGA

O-GlcNAc glycosylation of proteins is an important post-translational regulatory modification. The process is dynamic, and the protein O-GlcNAcase, encoded by the gene OGA, is responsible for removing the group again.

Inhibitors of OGA block cognitive decline and reduce number of amyloid plaques in animal models of Alzheimer's Disease (AD) (Yuzwa, Shan et al. 2014) and reduce amount of pathological Tau in the brain (Graham, Gray et al. 2014, Hastings, Wang et al. 2017). Furthermore, inhibition of OGA reduces cellular internalization of α-synuclein preformed fibrils and could be a strategy for Parkinson's Disease (PD) therapy (Tavassoly, Yue et al. 2021). Insertion of the chr10:101795374-101795480(−) and chr10:101795365-101795480(−) pseudoexon located in OGA intron 10 introduces a premature stop codon targeting the resulting transcript for degradation by the NMD pathway. By functionally mimicking OGA inhibition, SSO-mediated downregulation of OGA could be a promising approach for several neuropathies, including AD and PD.

Sweet Spots for OGA SSO targeting are shown in Table 3 and Table 6.
Graham, D. L., et al. 2 Neuropharmacology 79: 307-313.
Hastings, N. B., et al. 2017. Mol Neurodegener 12(1): 39.
Tavassoly, O., et al. 2021. FEBS J 288(2): 452-470.
Yuzwa, S. A., et al. 2014. Mol Neurodegener 9: 42.

### TMEM97

Transmembrane Protein 97 (TMEM97), also known as Sigma-2 receptor, plays an important role in cholesterol homeostasis. TMEM97 has been shown to be overexpressed in several cancers, and suppression of its expression inhibits glioma cancer cell growth and metastasis (Qiu, Sun et al. 2015). TMEM97 is also involved in the pathology of neurodegenerative diseases such as Alzheimer's Disease and its inhibition may be a potential therapy (Riad, Lengyel-Zhand et al. 2020). Inhibition of TMEM97 has also been proposed as a potential therapy for Niemann-Pick type C disease (Ebrahimi-Fakhari, Wahlster et al. 2016). SSO-mediated inclusion of the chr17:28320422-28320470(+) pseudoexon in TMEM97 intron 1 introduces a premature stop codon targeting the resulting transcript for degradation by the NMD pathway, and therefore downregulates TMEM97 gene expression.

Sweet Spot for TMEM97 SSO targeting are shown in Table 3 and Table 6.
Ebrahimi-Fakhari, D., et al. 2016. Hum Mol Genet 25(16): 3588-3599.
Qiu, G., et al. 2015. Tumour Biol 36(10): 8231-8238.
Riad, A., et al. 2020. Mol Neurobiol 57(9): 3803-3813.

TXNRD1

Thioredoxin Reductase 1 is encoded by TXNRD1 and is associated with unfavorable prognosis in patients with hepatocellular carcinoma (HCC) (Fu et al. 2017). In HCC tissues and cells, TXNRD1 is overexpressed and correlates positively with increasing clinical stage and shorter survival time (Fu et al. 2107). It has also been found to be mutated in several cancers, including HCC Jia et al. 2020). It is therefore a promising therapeutic target for target down-regulation.

Transcripts including the 158nt long pseudoexon located within intron 4 of the reference transcript are subject to degradation via the NMD system due to the introduction of a frame-shift and a resulting pre-mature termination codon. It may also lead to the production of a severely truncated protein lacking the active-site amino acids necessary for reductase activity. Both scenarios result in a complete loss of function of the gene product when the transcript includes the pseudoexon.

Sweet Spot for TXNRD1 targeting are shown in Table 3 and Table 6.
Fu B, et al. Biomed Res Int. 2017; 2017:4698167. doi: 10.1155/2017/4698167.
Jia Y, et al. Mol Clin Oncol. 2020 December; 13(6):83. doi: 10.3892/mco.2020.2153.

### SLC7A11

The solute-carrier SLC7A11 is a member of the cystine/glutamate transporter system Xc- and encodes xCT, which is overexpressed in many cancers, and is a marker of poor prognosis (reviewed in Lin et al. 2020). In glioblastoma this leads to increased glutamate secretion and neuronal death (Savaskan et al. 2008). Inhibition of xCT reduces neuronal death and edema, and prolongs survival in rats with gliomas (Savaskan et al. 2008). SLC7A11 upregulation also has an important cytoprotective effect in KRAS mutant cells by increasing intracellular antioxidant glutathione levels (Lim et al. 2019), and knock down of SLC7A11 strongly impairs growth of tumor xenografts Lim et al. 2019). SLC7A11 is a candidate therapeutic target for both KRAS-driven tumors that are typically highly therapy-resistant, and many other cancers including gliomas. While several xCT inhibitors exist, they are less specific than an SSO mediated downregulation of SLC7A11, and may lead to significantly more side effects when used in a clinical setting compared to an SSO based therapy.

Inclusion of the 56 nt pseudoexon located within intron 6 leads to introduction of a frame-shift and a resulting premature termination-codon, resulting in an NMD sensitive transcript, which may be down-regulated or express a truncated and non-functional protein.

Sweet Spot for SLC7A11 SSO targeting are shown in Table 3 and Table 6.

Therefore, treatment with the SSO to knock down SLC7A11 is a promising therapy for several cancers, offering higher specificity than current protein inhibitors with fewer side effects.
Lin W, et al. Am J Cancer Res. 2020 Oct. 1; 10(10):3106-3126.
Savaskan N E, et al. Nat Med. 2008 June; 14(6):629-32. doi: 10.1038/nm1772.
Lim J K M, et al. Mol Cell Oncol. 2019 Nov. 10; 7(1):1654814. doi: 10.1080/23723556.2019.1654814.
Lim J K M, et al. Proc Natl Acad Sci USA. 2019 May 7; 116(19):9433-9442. doi: 10.1073/pnas.1821323116.

### Cancers

The PEs in the known oncogenes STAT5B (de Araujo, Erdogan et al. 2019), MCCC2 (Chen, Zhang et al. 2021), UBAP2L (Li, Wang et al. 2018), SMYD2 (Li, Zhou et al. 2018), YBX1 (Xu, Li et al. 2017), PTPN11 (Chan, Kalaitzidis et al. 2008), DIAPH3 (Rong, Gao et al. 2020), COPS3 (Zhang, Yan et al. 2018), SNX5 (Zhou, Huang et al. 2020) and ZYG11A (Wang, Sun et al. 2016) all result in inclusion of an out of frame PE. Inclusion of these PEs will therefore lead to NMD mediated degradation of the oncogenic mRNA or production of a non-functional oncoprotein and are therefore suitable for treatment by Sweet Spot S SOs that activate inclusion SSO as cancer treatment.
Chan, G., et al. 2008. Cancer Metastasis Rev 27(2): 179-192.
Chen, et al. 2021. Cancer Cell Int 21(1): 22.
de Araujo, E. D., et al. 2019. Nat Commun 10(1): 2517.
Li, L. X., et al. 2018. Cell Death Dis 9(3): 326.
Li, Q., et al. 2018. Med Sci Monit 24: 7109-7118.
Namour, F., et al. 2012. Drugs R D 12(3): 141-163.
Rong, Y., et al. 2020. J Cell Mol Med. doi: 10.1111/jcmm.16196
Wang, X., et al. 2016. Oncotarget 7(7): 8029-8042.
Xu, L., et al. 2018. J Exp Clin Cancer Res 37(1): 135.
Zhou, Q., et al. 2020. Oncogene 39(10): 2140-2155.

### Example 9—HIF1A

### Aim of Study

Hypoxia inducible factor (HIF) is a transcription factor that is activated when there is a decrease in oxygen levels, or as a response to other environmental changes. HIF-1α contributes to tumor progression in cancer by promoting signalling for angiogenesis—the formation of new blood vessels forming from already exciting ones, invasiveness of the cells, metastasis and recruitment of immunosuppressive cells to the tumor environment (Tatrai et al 2014). Previous studies have shown that knock down of HIF-1α was able to reduce tumor mass and migration of cancer cells, and activation of the oncogene is highly correlated with the risk of metastases, making HIF-1α a possible target for anti-cancer therapy (Dai et al. 2011).

### Materials and Methods

We used the analysis pipeline (FIG. 2) to analyze publicly available RNA-seq data. Panc-1 cells were grown in RPMI and transfected with 40 nM SSO using Lipofectamine. After 24 hours, cells were harvested and RNA purified. RT-PCR was performed and the resulting product visualized on 2% agarose gel. In order to determine the optimal SSOs targeting the Sweet Spot region we tested several SSOs employing 25 nt long SSOs targeting the Sweet Spot region from +9 to +13 position downstream of the 5′ss of the PE. This showed that an SSO targeting from +10 (SEQ ID NO: 128) was superior in mediating pseudoexon inclusion into the HIF1A transcript. For hypoxia experiments transfected Panc-1 or U251 cells were first incubated at normoxic condition for 48 hours, then moved to a hypoxia chamber for 24 hours. To measure cell proliferation, viability and cytotoxicity with the optimal+10 SSO, WST-1 assay was carried out on cells under normoxia and hypoxia conditions. For protein extraction cell-lysates were rapidly frozen at −80C, to limit the time at normoxic conditions. Protein lysates were benzonase treated, and denatured proteins were separated on a 4-12% Nupage SDS-gel, and analysed by western blotting using antibodies against HIF1a and β-actin for control.

### SSOs:

All SSO were 25 nt long phosphorothioate RNA oligonucleotides with 2′-O-methyl modification on each sugar moiety (Produeced by LGC Biosearch Technologies). SSOs were used targeting position different positions inside the Sweet Spot region for that gene (relative to the 5′ splice site of the pseudoexon). Thus, the SSOs binds inside the Sweet Spot.

TABLE 4
SSO sequence 5′ -> 3′

| SEQ | | | |
|---|---|---|---|
| ID | | SSO | |
| NO | Gene | name | SSO sequence 5′ -> 3′ |
| 127 | HIF1A | SSO +9 | guccuucauaaacuaagcuugaauu |
| 128 | HIF1A | SSO +10 | aguccuucauaaacuaagcuugaau |
| 129 | HIF1A | SSO +11 | caguccuucauaaacuaagcuugaa |
| 130 | HIF1A | SSO +12 | ucaguccuucauaaacuaagcuuga |
| 131 | HIF1A | SSO +13 | uucaguccuucauaaacuaagcuug |

### Results

Using the analysis pipeline (FIG. 2) to analyze publicly available RNA-seq data we identified a 34 nt pseudoexon (chr14:61724198-61724230(+)) between exon 4 and 5 in HIF1A. The pseudoexon causes a frame-shift and introduces a stop codon (UAA) in exon 5. In order to determine the optimal SSOs targeting the Sweet Spot region we tested several SSOs employing 25 nt long SSOs targeting the Sweet Spot region from +9 to +13 position downstream of the 5′ss of the PE. This showed that an SSO targeting from +10 was superior in mediating pseudoexon inclusion into the HIF1A transcript. Transfecting U251 glioblastoma cells with the +10 SSOs resulted in up to 60% inclusion of the pseudoexon (FIG. 6A). Under normoxic conditions HIF-1α is degraded by hydroxylation, so to investigate the effect of the SSOs on a protein-level, Panc-1 cells were subjected to hypoxia, and protein extraction indicated that the +10 SSO efficiently decreased the level of HIF1A protein (FIG. 6B). WST-1 assay of U251 cells grown under hypoxic conditions had a lower viability when treated with the +10 SSO, than when treated with ctrl SSO (FIG. 6C).

### Conclusion

It can be observed that it is possible to optimize the binding site within the binding region of HIF1A. The SSO performing the best was “HIF1A+10” (SEQ ID NO: 128)

### Example 10—TRPM7

### Aim of Study

TRPM7 belongs to the protein super family Transient Receptor Potential (TRP), which conducts the traffic of different ions across membranes. The TRP proteins work as sensors and transducers which, when activated, leads to a transmembrane flow of ions that regulate associated pathways and various physiological responses (Liu et al 2014). TRPM7 can be cleaved by caspase, splitting the kinase domain from the pore in the membrane. Studies have shown that TRPM7 is highly expressed in brain tissue and deregulation of this channel is involved in neurodegenerative diseases such as amyotrophic lateral sclerosis (ALS), parkinsonism dementia and Alzheimer's disease. It was found that TRPM7 plays a critical role in neuronal death in cases of ischemia by mediating a Ca2+ influx causing calcium overload resulting in oxidative stress, nitric oxide production and cell death (Leng et al 2015; Sun et al. 2009). Knock down of TRPM7 inhibit delayed neuronal cell death, which is characteristic in Alzheimer's, Huntington's, Parkinson's disease and stroke patients. This suggests that knock down of TRPM7 could be a new therapy against neuronal disorders. TRPM7 also plays a significant role in several types of cancer including glioblastoma multiforme (GBM), retinoblastoma, nasopharyngeal carcinoma, leukemia, gastric, prostate, pancreatic, breast, head and neck cancers, and it is overexpressed in pancreatic and lung cancer cells. Finally, TRPM7 plays a role in diabetes, kidney disease, and inflammatory diseases.

### Materials and Methods

We used the analysis pipeline (FIG. 2) to analyze publicly available RNA-seq data. HeLa cells and U251 cells were grown in RPMI and transfected with 20 nM or nM SSO using Lipofectamine. After 24 hours, cells were harvested and RNA purified. RT-PCR was performed and the resulting product visualized on 2% agarose gel. For Incucyte experiments U251 cells were grown in 96-well plates transfected with SSOs at 20 and 40 nM and incubated in the incucyte instrument with images taken every 4th hour. A small SSO walk was performed employing 25 nt long SSOs targeting the Sweet Spot region from +9 to +13 position downstream of the 5′ss of the PE.

### SSOs:

All SSO were 25 nt long phosphorothioate RNA oligonucleotides with 2′-O-methyl modification on each sugar moiety (Produeced by LGC Biosearch Technologies). SSOs were used targeting position different positions inside the Sweet Spot region for that gene (relative to the 5′ splice site of the pseudoexon). Thus, the SSOs bind inside the Sweet Spot (see Table 5).

TABLE 5

| SEQ | | | |
|---|---|---|---|
| ID | | SSO | |
| NO | Gene | name | SSO sequence 5′ -> 3′ |
| 132 | TRPM7 | SSO +9 | uucugacuucugcauauacuaaaga |
| 133 | TRPM7 | SSO +10 | uuucugacuucugcauauacuaaag |
| 134 | TRPM7 | SSO +11 | cuuucugacuucugcauauacuaaa |
| 135 | TRPM7 | SSO +12 | acuuucugacuucugcauauacuaa |
| 136 | TRPM7 | SSO +13 | gacuuucugacuucugcauauacua |

### Results

Using the analysis pipeline (FIG. 2) we identified a 60 nt pseudoexon (chr15:50588192-50588250(−)) located between exon 27 and 28 in TRPM7. The pseudoexon results in introduction of 20 amino acids in the region of the cleavage site between the channel-domain and the kinase domain of the TRMP7 protein. In order to determine the optimal SSOs targeting the Sweet Spot region we tested several 25 nt long SSOs targeting the Sweet Spot region from +9 to +13 position downstream of the 5′ss of the PE (FIGS. 5A and B). This showed that an SSO targeting from +13 was superior in mediating pseudoexon inclusion into the TRPM7 transcript. Transfection of HeLa cells or U251 glioblastoma cells with the optimal+13 targeting SSO resulted in high inclusion of the pseudoexon, which decreased growth and proliferation of cancer cells (FIG. 5C).

### Conclusion

The normal expression of the TRPM7 protein is most efficiently decreased by using the SSO that binds from the +13 position and mediates a high level of pseudoexon inclusion.

### REFERENCES

Tátrai E, et al. Oncotarget 2017, 8:44498-44510.
Dai Y, et al. International Journal of Radiation Oncology*Biology*Physics 2011, 81:521-528.
Liu M, et al. Cell Signal 2014, 26:2773-2781.
Leng T D, et al. CNS Neurosci Ther 2015, 21:252-261.
Sun H S, et al. Nat Neurosci 2009, 12:1300-1307.

### Example 11—Further Relevant Targets Experimentally Validated

The following Sweet Spot sequences were identified in disease associated genes using the criteria according to the invention (see e.g. example 2) and demonstrated by functional testing to be targets for SSOs, allowing for incorporation of the pseudoexon in the mature mRNA.

### Materials and Methods

We used public RNA-sequencing data (Geuvadis, E-MTAB-2836, E-MTAB-513, GSE52946, and GSE124439) and mapped them with STAR after trimming for adapter contamination and poor quality bases with bbduk. HeLa cells were seeded in 12-well plates and forward transfected at 60% confluence with 20 or 40 nM 2′-O-methyl SSOs with full phosphorothioate backbone using Lipofectamine RNAiMAX (invitrogen). A non-binding ctrl SSO (5′GCUCAAUAUGCUACUGCCAUGCUUG3′) (SEQ ID NO: 126) was used as control. RNA was harvested after 48 hours using Trizol (Invitrogen) and chloroform to isolate the RNA, followed by precipitation with isopropanol. Complementary DNA (cDNA) was synthesized from 500 ng RNA using the High capacity cDNA kit (Applied Biosystems). Primers were designed to span at least one exon-exon junction of the neighboring exons flanking the pseudoexons of interest. PCR was carried out using TEMPase Hot Start DNA polymerase (ampliqon) and 1 μl cDNA per reaction. 0.5 pmol/μl of each primer was used. The PCR products were separated on a 2% Seakem LE (Lonza) TBE agarose gel, for 1 hour at 80V.

The Sweet Spot region is located+9 to +39 of the 5′ splice site of the pseudoexon.

All SSOs were 25 nt long phosphorothioate RNA oligonucleotides with 2′-O-methyl modification on each sugar moiety (Produced by LGC Biosearch Technologies). SSOs were used targeting position+11 to +35 inside the Sweet Spot region for that gene (relative to the 5′ splice site of the pseudoexon). Thus, the SSOs bind inside the Sweet Spot.

### Results

Using our double-junction approach, we identified further 46 fully spliced pseudoexons and tested SSOs targeting the Sweet Spot region from +11 to +35 downstream of the selected pseudoexons identified in RNA sequencing data (table 6). All 46 of these selected SSOs were able to increase pseudoexon inclusion into the mRNA transcript of the targeted gene (Table 6 below).

Table 6: 46 pseudoexons matching all criteria, all activated by an SSO located within the Sweet Spot region. Sweet Spot region is annotated by its genomic sequence (DNA).

TABLE 6
Targets functionally validated as responsive to SSO targeting
the Sweet Spot sequence.

| SSO | | hg38 | |
|---|---|---|---|
| seq | | pseudoexon | |
| ID | Gene | coordinates | Sweet Spot seq (+9 to +39) |
| 79* | ROCK1 | chr18: 21017021- | CTGTGAAGGCCGTGAGGTAAGAGACCTT |
| | | 21017098(−) | GAC |
| 80* | ROCK1 | chr18: 21022445- | GGAATGGGGGAAATGGAGAATAACCAGT |
| | | 21022564(−) | ATA |
| 81* | OGA | chr10: 101795365- | AAGGGCGGTAGACATGAACTGAAGTCAC |
| | | 101795480(−) | GTT |
| 82* | OGA | chr10: 101795374- | GGTAGATTTAAGGGCGGTAGACATGAAC |
| | | 101795480(−) | TGA |
| 83* | TMEM97 | chr17: 28320422- | CGTGTAGCAAGTTGCAGTCTGGGGACTT |
| | | 28320470(+) | GGT |
| 85* | SLC7A11 | chr4: 138191360- | TAGATTAGACATCAGTGGTATTGAAATTT |
| | | 138191415(−) | AA |
| 87* | MAPKAPK | chr12: 111870683- | CTTATAGAGTGAAGGGTCCCTAGGCCAA |
| | 5 | 111870782(+) | GAC |
| 90* | MCCC2 | chr5: 71642612- | CAGTGCCATTTAGGATGTACTGCATAAGT |
| | | 71642727(+) | TT |
| 91* | MCCC2 | chr5: 71642664- | CAGTGCCATTTAGGATGTACTGCATAAGT |
| | | 71642727(+) | TT |
| 93* | DIAPH3 | chr13: 59923001- | TAGCATTAACAGTATAGGTAGGGAATCTG |
| | | 59923041(−) | GT |
| 94* | DIAPH3 | chr13: 59923001- | TAGCATTAACAGTATAGGTAGGGAATCTG |
| | | 59923111(−) | GT |
| 99* | PTPN11 | chr12: 112456115 | TCAATGGATGTGCTAGCCGCTCCATTTGG |
| | | -112456268(+) | CT |
| 100* | UBAP2L | chr1: 154241700- | AATATCTGATTGTCGGATTTATCCCAGGA |
| | | 154241797(+) | AG |
| 105* | LRIG2 | chr1: 113116745- | TCAGTAAAGTTTAAAGGGTAGCTAGGAGC |
| | | 113116798(+) | CA |
| 106* | RNF115 | chr1: 145784178- | CAGGCCAGGATAAGTTGTCGAAGTCACA |
| | | 145784251(−) | ATG |
| 107* | HGS | chr17: 81684813- | GTGCTTCAGGGATGAATCCAGAGGTTAAC |
| | | 81684891(+) | TA |
| 110* | HMGCS1 | chr5: 43297529- | TGTCATCCCCACTTTACAGAGAAGTGGGT |
| | | 43297673(−) | TA |
| 111* | SND1 | chr7: 127725671- | GCCTTTTCCAAACCAAAGAACAAAGCGGG |
| | | 127725726(+) | AG |
| 114* | KNTC1 | chr12: 122595478 | AGGACTATAGGCTGCGCATAGCTCGAAG |
| | | -122595600(+) | GAG |
| 117* | THOC2 | chrX: 123706588- | GTAGGGAGAAAAAAAAAAGCCTGTGCTG |
| | | 123706704(−) | TAA |
| 118* | SMC1A | chrX: 53402287- | GATGAACACAACACAGTCTTTGCTGAAGG |
| | | 53402379(−) | AG |
| 122* | PHLPP1 | chr18: 62885960- | ACTAGTAGATCACACCCCTAGATAGTTTA |
| | | 62886035(+) | GC |
| 123* | AURKA | chr20: 56390967- | AATTGGAGCAAATGCCTGTAGCTTCTGTC |
| | | 56391068(−) | AG |
| 137 | ATXN7 | chr3: 63921276- | TTTAACCAGTGGTCCCCAGGCTTTATATTT |
| | | 63921329(+) | C |
| 138 | DDR2 | chr1: 162666267- | TTGTGGAATGAATGAATGAGCAAATGAAG |
| | | 162666368(+) | GA |
| 139 | HIF1A | chr14: 61724148- | AATTCAAGCTTAGTTTATGAAGGACTGAA |
| | | 61724230(+) | CA |
| 140 | HTT | chr4: 3223670- | GAAGCTGGAAACGTGACAGGAACTGACG |
| | | 3223729(+) | TGG |
| 141 | LRRK2 | chr12: 40362438- | AAAATACATTGTCCTCATCCTTATGAAATT |
| | | 40362491(+) | A |
| 142 | LRRK2 | chr12: 40362410- | AAAATACATTGTCCTCATCCTTATGAAATT |
| | | 40362491(+) | A |
| 143 | RAP1GDS | chr4: 98419239- | TACTGCACCAGGCCTTAGGCATCTTTAGT |
| | 1 | 98419331(+) | TG |
| 144 | TTBK2 | chr15: 42809105- | TGGGTGTGTTCTAATGACTAGTATCAATG |
| | | 42809208(−) | TG |
| 145 | TTK | chr6: 80034193- | CCATAAAACTAACAAATCACAACTCTGAC |
| | | 80034275(+) | AC |
| 146 | BUD31 | chr7: 99419162- | CTATATGGCATGGTGGCAGGTCCTTCGTG |
| | | 99419275(+) | GG |
| 147 | CD44 | chr11: 35188146- | TTGTCCCTGACCTGTGCCTTGCAATAGGA |
| | | 35188238(+) | AG |
| 148 | CDKL5 | chrX: 18623879- | TTTTATGTATGGTAGCCCTGAAAACTGCT |
| | | 18624001(+) | CC |
| 149 | ITGBL1 | chr13: 101705140- | GTGGTATAACCCAGGGAAAATCTCTCTTA |
| | | 101705240(+) | TT |
| 150 | LRP6 | chr12: 12149294- | AAGAAATACTCCTTAAGACTTGAGAGAGC |
| | | 12149360(−) | CT |
| 151 | MELK | chr9: 36654164- | AGAAAGCTATTAGATACGTATACCTCTAT |
| | | 36654224(+) | GC |
| 152 | PDE4D | chr5: 59036523- | TGCCACACCTTCAGTCTGAACTGGAAATG |
| | | 59036587(−) | TG |
| 153 | PMPCB | chr7: 103305205- | AGAAGGTTTCCTTGAGGTCATGTTTGAAG |
| | | 103305273(+) | CC |
| 154 | RBPJ | chr4: 26388182- | CACGGTGCCCAGCCTAAAACAGTTATTAT |
| | | 26388277(+) | AT |
| 155 | ROCK2 | chr2: 11198121- | CTAGATACTCTTCACTGTTGTTGCACATA |
| | | 11198215(−) | GA |
| 156 | TASP1 | chr20: 13415123- | GCATCTTAAAATATTTAAAATTTAAAAACT |
| | | 13415262(−) | A |
| 157 | ATP5F1C | chr10: 7805451- | ATTGAGACCATCCTGGCTAACTCGGTGAA |
| | | 7805532(+) | AC |
| 158 | LRRK2 | chr12: 40362438- | AAAATACATTGTCCTCATCTTTATGAAATT |
| | | 40362491(+) | A |
| 159 | LRRK2 | chr12: 40362410- | AAAATACATTGTCCTCATCTTTATGAAATT |
| | | 40362491(+) | A |

*Also included in table 3.
Underlining indicates tested binding sequence of the SSO.
Binding is to the corresponding pre-mRNA.
hg38 Genome coordinates follow GFF/GTF format; one-based start and end coordinates.

In Table 6, the Sweet Spots for SEQ ID 141 and 142 correspond to the major allele (C) of rs17444202. The Sweet Spots for SEQ ID 158 and 159 correspond to the minor allele (T) of rs17444202.

Seq ID NO's: 160-180 in Table 7 are further pseudoexons in genes where at least one other pseudoexon has already been activated by a SSO targeting the Sweet Spot (genes listed in table 1 and table 6). Seq ID NO's: 181-201 in Table 7 are additional Sweet Spot sequences identified using the criteria according to the invention (see e.g. example 2). Thus, these targets will with very high plausibility be functional targets for SSOs, allowing for incorporation of the pseudoexon in the mature mRNA.

TABLE 7
relevant target pseudoexons.

| SSO | | | |
|---|---|---|---|
| seq | | hg38 | |
| ID | Gene | coordinates | Sweet Spot seq |
| 160 | RNF115 | chr1: 145794074- | TTCGAGACCAGCCTGGCCATCGTGGCGAA |
| | | 145794196(−) | AC |
| 161 | RNF115 | chr1: 145784113- | TTCCAGGGCTTACCTCTAGTCAACACAGTA |
| | | 145784251(−) | G |
| 162 | RNF115 | chr1: 145780773- | AAGAGGGCAGGGTGCTCTGAGCTGATTTA |
| | | 145780881(−) | TA |
| 163 | RNF115 | chr1: 145759389- | GGAGGAAAATATTTAAGAGTTCATTCAGTT |
| | | 145759535(−) | T |
| 164 | LRP6 | chr12: 12184211- | TTTGGCTTATCACAGTTGTTCATGTTATGG |
| | | 12184244(−) | T |
| 165 | LRP6 | chr12: 12212260- | AGACCATTATTAAATATTTGAGTGCTGACT |
| | | 12212305(−) | G |
| 166 | LRP6 | chr12: 12197784- | CACCATGCCTGGCCATAAATTTAATTCTTA |
| | | 12197878(−) | T |
| 167 | UBAP2L | chr1: 154249958- | TCACTTAATGTCATTGATAGGTTCTTGCGA |
| | | 154250030(+) | C |
| 168 | UBAP2L | chr1: 154249997- | TCACTTAATGTCATTGATAGGTTCTTGCGA |
| | | 154250030(+) | C |
| 169 | UBAP2L | chr1: 154222819- | CAAGATTCTTCATCTCCAGTGTGATAGCTG |
| | | 154222986(+) | G |
| 170 | UBAP2L | chr1: 154241738- | AATATCTGATTGTCGGATTTATCCCAGGAA |
| | | 154241797(+) | G |
| 171 | UBAP2L | chr1: 154262173- | GACCATACTTAATAGAGTGGGAATTGGAC |
| | | 154262202(+) | TG |
| 172 | ZNF558 | chr19: 8831564- | AGTCATGGCCCACAGGCTGGGCCCTTATT |
| | | 8831582(−) | AC |
| 173 | ZNF558 | chr19: 8831564- | AGTCATGGCCCACAGGCTGGGCCCTTATT |
| | | 8831647(−) | AC |
| 174 | ZNF558 | chr19: 8831564- | AGTCATGGCCCACAGGCTGGGCCCTTATT |
| | | 8831642(−) | AC |
| 175 | KNTC1 | chr12: 122585408- | ATGAGGCCACTTCTCTGTTCCAGTCACTTG |
| | | 122585420(+) | A |
| 176 | KNTC1 | chr12: 122564657- | CCCAGTGCTCATTTCTTTAACACAAATATT |
| | | 122564756(+) | T |
| 177 | KNTC1 | chr12: 122621236 | ACCAGTAGATAGAGATCTACATAAATGAAC |
| | | -122621288(+) | A |
| 178 | KNTC1 | chr12: 122620698 | GAAAGATAGATTAAGGTCCAGCTGAGGGC |
| | | -122620759(+) | TG |
| 179 | KNTC1 | chr12: 122554273 | CACCATACCCAGCAAGAATAATTCTGTGTA |
| | | -122554359(+) | T |
| 180 | KNTC1 | chr12: 122528435 | AACTAAATTAGTCCCTTGGTATTGGTATCC |
| | | -122528583(+) | A |
| 181 | SMC1A | chrX: 53404716- | TTGGAGACCAGCCTGGCCAAACCTGTCTC |
| | | 53404804(−) | TA |
| 182 | SMC1A | chrX: 53407427- | GAAGAGATGTGAAGCTTCCATGCCCAATC |
| | | 53407474(−) | TG |
| 183 | SMC1A | chrX: 53407371- | ACACAGGGGCTTCATTATGTAGTCATGATT |
| | | 53407474(−) | G |
| 184 | FLT1 | chr13: 28337518- | CTCTGATTGATTAACGTGGGAGCAAGGAG |
| | | 28337637(−) | GC |
| 185 | FLT1 | chr13: 28414636- | CTCTCGATGAAATCAGATAATAAACTGACA |
| | | 28414760(−) | A |
| 186 | FLT1 | chr13: 28435084- | TGAGTTAATGTTATGAATCTTGGAGGACCT |
| | | 28435140(−) | G |
| 187 | FLT1 | chr13: 28435084- | TGAGTTAATGTTATGAATCTTGGAGGACCT |
| | | 28435205(−) | G |
| 188 | EFEMP1 | chr2: 55870349- | CACTAATCATTGATGGTTAATTAATTATAC |
| | | 55870498(−) | A |
| 189 | CLCN1 | chr7: 143319065- | TAAAGGAAAGAGGTTTAATGGGCCCACAG |
| | | 143319127(+) | TT |
| 190 | CD5L | chr1: 157836389- | CTAATTATCTAGCTGTGGGAAGAAGTAGA |
| | | 157836492(−) | TG |
| 191 | A2M | chr12: 9072149- | AAGTAGAGTTTCTTGACAGGGTACCATTAA |
| | | 9072182(−) | G |
| 192 | MAOB | chrX: 43778486- | TTCCCTCAATAACAACTAAGTAAATATTAC |
| | | 43778558(−) | C |
| 193 | MAOB | chrX: 43837770- | ACTTGCAATATGCTGTGGTTTAGGTGATGC |
| | | 43837923(−) | T |
| 194 | HSP90AA1 | chr14: 102117585 | CACAGGAACTTATTCCTCACTGGTAGAAAA |
| | | -102117727(−) | C |
| 195 | ALK | chr2: 29253737- | TTTCTGCAGCTGGGGGGTGTTCAGCTCCT |
| | | 29253814(−) | AG |
| 196 | JPH3 | chr16: 87695507- | AGCCTGGGTCTGCAGAACAGGTTTCAAGG |
| | | 87695657(+) | AA |
| 197 | HNF1B | chr17: 37711993- | TCTGTAGGGCAGCACCCACCCCTGCCGTC |
| | | 37712084(−) | CT |
| 198 | P2RX7 | chr12: 121149278- | TAAAGGAAAGAGGTTTAATGGGCCCACAG |
| | | 121149369(+) | TT |
| 199 | P2RX7 | chr12: 121175867- | CAGCACCTTCGATCTGACCAGTTTAGCAAC |
| | | 121175980(+) | C |
| 200 | DBI | chr2: 119369029- | TACAGTGAGTTCGTAAACTCTGTCCTTCCA |
| | | 119369176(+) | G |
| 201 | DBI | chr2: 119371982- | GATACTGTCTCCTGTAATTAGTAGAATCTC |
| | | 119372085(+) | A |

Underlining indicates tested binding sequence of the SSO.
Binding is to the corresponding pre-mRNA.
hg38 Genome coordinates follow GFF/GTF format; one-based start and end coordinates.

TABLE 8
SSOs targeting SMAD2 and LRRK2

| SEQ ID | | | |
|---|---|---|---|
| NO | Gene | SSO name | SSO sequence 5′ -> 3′ |
| 202 | SMAD2 | SMAD2SSO+9 | UCUCGCUUAGCUUCAACAACUAAGAC |
| 203 | SMAD2 | SMAD2SSO+10 | AUCUCGCUUAGCUUCAACAACUAAGA |
| 204 | SMAD2 | SMAD2SSO+11 | CAUCUCGCUUAGCUUCAACAACUAAG |
| 205 | SMAD2 | SMAD2SSO+12 | GCAUCUCGCUUAGCUUCAACAACUAA |
| 206 | SMAD2 | SMAD2SSO+13 | UGCAUCUCGCUUAGCUUCAACAACUA |
| 207 | SMAD2 | SMAD2SSO+14 | CUGCAUCUCGCUUAGCUUCAACAACU |
| 208 | LRRK2 | LRRK2SSO+11 | UUCAUAAAGACGAGGACAAUGUAUU |
| 209 | LRRK2 | LRRK11CSSOC | UUCAUAAGGACGAGGACAAUGUAUU |
| 210 | LRRK2 | LRRK11CSSOT | UUCAUAAAGACGAGGACAAUGUAUU |
| 211 | LRRK2 | LRRK9CSSOC | CAUAAGGACGAGGACAAUGUAUUUU |
| 212 | LRRK2 | LRRK9CSSOT | CAUAAAGACGAGGACAAUGUAUUUU |
| 213 | LRRK2 | LRRK13CSSOC | AUUUCAUAAGGACGAGGACAAUGUA |
| 214 | LRRK2 | LRRK13CSSOT | AUUUCAUAAAGACGAGGACAAUGUA |
| 215 | LRRK2 | LRRK11SSOC | UUCAUAAGGAUGAGGACAAUGUAUU |
| 216 | LRRK2 | LRRK11SSOT | UUCAUAAAGAUGAGGACAAUGUAUU |

### Example 12—RNF115

RNF115 (Ring Finger Protein 115), previously named Breast Cancer Associated 2 (BCA2), is a RING-finger E3 ubiquitin that mediates polyubiquitination of substrates. RNF115 causes ubiquitination and proteasomal degradation of the tumor suppressor p21 in breast cancer (Wang et al. 2013). In lung cancer RNF115 also functions as an oncogene by regulating Wnt/β-catenin pathway via ubiquitination of adenomatous polyposis coli (APC) leading to increased proliferation (Wu et al. 2021).

RNF115 is associated with breast cancer. It is overexpressed in more than 50% of invasive breast cancers, and it's up-regulation correlates with estrogen receptor positive (ER+) status and poor prognosis.

Therefore, inhibiting RNF115 activity will inhibit the β-catenin and function to inhibit cancers, such as lung cancer and breast cancer.

Through in silico analysis and in vivo experiments, we aimed at investigating the presence of pseudoexons in the RNF115 gene, which may be used to down-regulate the expression this gene.

### Materials and Methods

We used our novel double-junction approach to identify fragments with fully spliced pseudoexons in publicly available RNA-seq data from the GEUVADIS consortium, E-MTAB-2836, E-MTAB-513, GSE52946, and GSE124439. HeLa cells, NCI-H23 lung cancer cells and NCI-H23 lung cancer cells were seeded in 12-well plates and forward transfected at 60% confluence with 5 nM, 10 nM or 20 nM SSO using Lipofectamine RNAiMAX (invitrogen).

SSO (targeting SEQ ID NO: 106; specific target sequence is underlined in SEQ ID NO: 106 in table 3 and table 6) was a 25 nt long phosphorothioate RNA oligonucleotides with 2′-O-methyl modification on each sugar moiety (Produced by LGC Biosearch Technologies). The SSO is complementary to position+11 to +35 inside the Sweet Spot region for the RNF115 gene (relative to the 5′ splice site of the pseudoexon). Thus, the SSOs binds inside the Sweet Spot.

A non-binding SSO (5′GCUCAAUAUGCUACUGCCAUGCUUG3′) (SEQ ID NO: 126) with similar modifications was used as a negative control. RNA was harvested after 48 hours using Trizol (Invitrogen) and chloroform to isolate the RNA, followed by precipitation with isopropanol. Complementary DNA (cDNA) was synthesized from 500 ng RNA using the High capacity cDNA kit (Applied Biosystems). Primers were designed to span at least one exon-exon junction of the neighboring exons flanking the pseudoexons of interest. PCR was carried out using TEMPase Hot Start DNA polymerase (ampliqon) and 1 μl cDNA per reaction. 0.5 pmol/μl of each primer was used. The PCR products were separated on a 2% Seakem LE (Lonza) TBE agarose gel, for 1 hour at 80V. Protein was harvested after 72 hours for western blotting and denatured proteins were separated on a 4-12% NuPAGE SDS-gel. Western blot analysis was performed with antibodies directed towards RNF115 (ab187642, Abcam) β-catenin (#9587, Cell Signaling Technology), β-actin for control (ab8229, Abcam).

To measure cell proliferation, viability and cytotoxicity with the optimal +11 SSO, NCI-H23 cells were reverse transfected with SSOs in a concentration gradient, and WST-1 assay (Roche) was carried out on cells 48 hours after transfection.

### Results

Using our double-junction approach to examine fragments with fully spliced pseudoexons, we identified a pseudoexon within intron 3 of the RNF115 gene. Inclusion of the chr1:145784178-145784251 RNF115 pseudoexon introduces 74 bp between exon 3 and 4 in the mRNA. This will lead to a reading frame-shift and a resulting pre-mature termination codon leading to degradation via the NMD system. It will also lead to the production of a severely truncated protein (of only 84 amino acids) lacking the functional domains. This truncated protein could function as a decoy by binding to substrates and inhibit polyubiquitination and function to inhibit cancers (Table 3 and Table 6).

Transfection of NCI-H23 cancer cells with an SSO complementary to position+11 to +35 downstream of the 5′ splice site (SEQ ID NO: 106) resulted in up to 90% more inclusion of the pseudoexon (FIG. 7A).

Growth of NCI-H23 cancer cells is inhibited by treatment by SSO-mediated down-regulation as shown by WST-1 assay (FIG. 7B). Furthermore, RNF115 protein levels are severely decreased following treatment of NCI-H23 lung cancer cells as shown by western blotting (FIG. 7C). The decreased levels of functional RNF115 caused decreased levels of β-catenin as shown by western blotting (FIG. 7C).

### Conclusion

The normal expression of the RNF115 gene product is decreased by at least 90% using a specific SSO to increase inclusion of a pseudoexon, which disrupts the function of the normal gene product. This reduces β-actin expression and growth of lung cancer cells showing SSO based activation of the RNF115 pseudoexon works to inhibit cancer.

Similarly, inclusion of the chr1: 145794075-145794196 RNF115 pseudoexon introduces 122 bp between exon 1 and 2 in the mRNA. This will lead to a reading frame-shift and a resulting pre-mature termination codon leading to degradation via the NMD system. It will also lead to the production of a severely truncated protein lacking the functional domains. This truncated protein could function as a decoy by binding to substrates and inhibit polyubiquitination and function to inhibit cancers (Table 7).

Similarly, inclusion of the chr1: 145784114-145784251 RNF115 pseudoexon introduces 138 bp between exon 3 and 4 in the mRNA. This will lead to a premature stop codon only twelve codons downstream of glutamic acid 73 leading to degradation via the NMD system. It will also lead to the production of a severely truncated protein (of only 84 amino acids) lacking the functional domains. This truncated protein could function as a decoy by binding to substrates and inhibit polyubiquitination and function to inhibit cancers (Table 7).

Similarly, inclusion of the chr1: 145780774-145780881 RNF115 pseudoexon introduces 108 bp between exon 3 and 4 in the mRNA. This will lead to a premature stop codon only twenty-four codons downstream of glutamic acid 73 leading to degradation via the NMD system. It will also lead to the production of a severely truncated protein (of only 96 amino acids) lacking the functional domains. This truncated protein could function as a decoy by binding to substrates and inhibit polyubiquitination and function to inhibit cancers (Table 7).

Similarly, inclusion of the chr1: 145759390-145759535 RNF115 pseudoexon introduces 146 bp between exon 4 and 5 in the mRNA. This will lead to a reading frame-shift after glycine 143 and a resulting pre-mature termination codon 94 codons downstream leading to degradation via the NMD system. It will also lead to the production of a severely truncated protein (of 226 amino acids) lacking the functional domains. This truncated protein could function as a decoy by binding to substrates and inhibit polyubiquitination and function to inhibit cancers (Table 7).
Wang Z, Nie Z, Chen W, Zhou Z, Kong Q, Seth A K, et al. (2013) RNF115/BCA2 E3 ubiquitin ligase promotes breast cancer cell proliferation through targeting p21Waf1/Cip1 for ubiquitin-mediated degradation. Neoplasia. 15(9):1028-35.
Wu X T, Wang Y H, Cai X Y, Dong Y, Cui Q, Zhou Y N, Yang X W, Lu W F, Zhang M. (2021) RNF115 promotes lung adenocarcinoma through Wnt/β-catenin pathway activation by mediating APC ubiquitination. Cancer Metab. 9(1):7.

### Example 13—LRRK2

### Aim of Study

Parkinson's disease is a progressive neurodegenerative disorder characterized by loss of dopaminergic neurons that affects movement control. Mutations in the leucine-rich repeat kinase 2 (LRRK2) gene account for common risk factors associated with Parkinson's disease (Alessi & Sammler, 2018). Dominantly inherited and sporadic pathogenic mutations in LRRK2 causes hyperactivation of the LRRK2 kinase, and downregulation of LRRK2 gene expression is a potential treatment strategy.

### Materials and Methods

We used the analysis pipeline (FIG. 2) to identify fragments with fully spliced pseudoexons in publicly available RNA-seq data. HeLa and U251 cells were reverse transfected with 20 nM SSO (targeting SEQ ID NO: 141 and SEQ ID NO: 142; specific target sequence is underlined in SEQ ID NO: 141 and SEQ ID NO: 142 in table 6) using Lipofectamine RNAiMAX (invitrogen). A non-targeting SSO (5′-GCUCAAUAUGCUACUGCCAUGCUUG-3′) (SEQ ID NO: 126) was used as a negative control. Cells were harvested after 48 hours using Trizol (Invitrogen) and RNA was extracted using chloroform and isopropanol. Complementary DNA (cDNA) was synthesized using the High-Capacity cDNA Reverse Transcription Kit (Applied Biosystems) and PCR was carried out using the TEMPase Hot Start DNA Polymerase (Ampliqon) with primers in LRRK2 exon 47 and 48. The PCR products were separated on a 1.5% SeaKem LE (Lonza) TBE agarose gel.

### Results

Using our analysis pipeline (FIG. 2), we identified two LRRK2 pseudoexons that can be activated by an SSO targeting the Sweet Spot. The two pseudoexons; chr12:40362438-40362491(+) and chr12:40362410-40362491(+), are located within intron 47 and have the same 5′ splice site. Transfection of HeLa and U251 cells with an SSO complementary to position+11 to +35 downstream of the 5′ splice site (SEQ ID NO: 215) resulted in increased inclusion of both pseudoexons between exon 47 and 48 in the LRRK2 mRNA transcript (FIG. 9). The chr12:40362438-40362491(+) LRRK2 pseudoexon is 54 nt long and pseudoexon inclusion will introduce 18 amino acids to the WD40 domain of the translated LRRK2 protein. The chr12:40362410-40362491(+) LRRK2 pseudoexon is 82 nt long and pseudoexon inclusion will cause a frame-shift and insertion of a premature termination codon, and target the transcript for degradation by nonsense-mediated mRNA decay.

It was surprisingly discovered that the identified Sweet Spot sequence harbors a SNP, rs17444202. We therefore used SSOs targeting either SEQ ID NO: 141 and 142 corresponding to the major allele (C) of rs17444202 or SSOs targeting SEQ ID 158 and 159 corresponding to the minor allele (T) of rs17444202 (see table 6 in example 11). Further, we used SSOs with additional mismatches to achieve preferential targeting of either the major or minor allele. All SSOs induced inclusion of the chr12:40362438-40362491(+) LRRK2 pseudoexon and the chr12:40362410-40362491(+) LRRK2 pseudoexon (results not shown). The sequences of the employed SSOs are listed in table 8 in example 11, with SEQ ID NO's: 208-216.

### Conclusion

The normal and hyper-activated function of LRRK2 will be decreased by using a specific SSO targeting the Sweet Spot to induce pseudoexon inclusion and thereby reduce the expression and activity of the normal LRRK2 gene product. Pseudoexon inclusion that introduces amino acids to the translated sequence will potentially reduce gene expression by disruption of protein function or alter normal protein function. Pseudoexon inclusion that causes frame-shift with insertion of a premature termination codon will reduce gene expression by degradation of the transcript or translation of a truncated and non-functional protein.

Without being bound by theory, by the identification of a SNP in the Sweet Spot region it is possible to make allele-specific targeting (or at least allele-preferred targeting), by screening the subjects SNP status before selecting SSOs and initiating a treatment.
Reference: Alessi, D. R. & Sammler E (2018) Science, 360(6384):36-37. DOI: 10.1126/science.aar5683

### Example 14—LRP6

LRP6 (LDL Receptor Related Protein 6) is a member of the low density lipoprotein (LDL) receptor gene family. LRP6 functions as a receptor and co-receptor for Wnt in the Wnt/beta-catenin signaling cascade and plays a role in the regulation of cell differentiation, proliferation, and migration. It is also involved in glucose and lipid metabolism signaling. Inhibition of LRP6 can be a therapeutic option for cancers, such as breast-, liver- and colorectal-cancer, as well as metabolic and neurodegenerative disease (Reviewed by Jeong and Jho 2021).

Inclusion of the chr12:12149294-12149360(−) LRP6 pseudoexon introduces 67 bp between exon 13 and 14 in the mRNA. This will lead to a reading frame-shift and a resulting pre-mature termination codon leading to degradation via the NMD system. It will also lead to the production of a severely truncated protein (of only 1006 amino acids) lacking the transmembrane and cytosolic domains. This truncated protein could function as a decoy receptor for Wnt proteins and thereby inhibit Wnt signaling and function to inhibit cancers and other diseases. (Table 6—Functionally validated)

Similarly, inclusion of chr12:12184211-12184244(−) LRP6 pseudoexon will introduce 34 bp between exon 4 and 5 in the mRNA. This will lead to a reading frame-shift and a resulting pre-mature termination codon leading to degradation via the NMD system. It will also lead to the production of a severely truncated protein lacking the transmembrane and cytosolic domains. This truncated protein could function as a decoy receptor for Wnt proteins and thereby inhibit Wnt signaling and function to inhibit cancers and other diseases (Table 7).

Similarly, inclusion of chr12:12212260-12212305(−) LRP6 pseudoexon will introduce 46 bp between exon 1 and 2 in the mRNA. This will lead to a reading frame-shift and a resulting pre-mature termination codon leading to degradation via the NMD system. It will also lead to the production of a severely truncated protein lacking the transmembrane and cytosolic domains. This truncated protein could function as a decoy receptor for Wnt proteins and thereby inhibit Wnt signaling and function to inhibit cancers and other diseases (Table 7).

Similarly, inclusion of chr12:12197784-12197878(−) LRP6 pseudoexon will introduce 95 bp between exon 3 and 4 in the mRNA. This will lead to a reading frame-shift and a resulting pre-mature termination codon leading to degradation via the NMD system. It will also lead to the production of a severely truncated protein lacking the transmembrane and cytosolic domains. This truncated protein could function as a decoy receptor for Wnt proteins and thereby inhibit Wnt signaling and function to inhibit cancers and other diseases (Table 7).

### Example 15—LRRK2—SNP

### Aim of Study

Parkinson's disease is a progressive neurodegenerative disorder characterized by loss of dopaminergic neurons that affects movement control. Mutations in the leucine-rich repeat kinase 2 (LRRK2) gene account for common risk factors associated with Parkinson's disease (Alessi & Sammler, 2018). Dominantly inherited and sporadic pathogenic mutations in LRRK2 causes hyperactivation of the LRRK2 kinase, and downregulation of LRRK2 gene expression is a potential treatment strategy. Allele-specific targeting is particularly promising for downregulation of the hyperactivated disease allele with minimal or no effect on the wild type.

### Materials and Methods

We used RNA-seq analysis to identify fragments with fully included pseudoexons in proprietary and publicly available RNA-seq data, and we used the dbSNP database (https://www.ncbi.nlm.nih.gov/SNP) to identify SNPs that strengthens the 3′ or 5′ splice sites (increases the MaxEnt scores). Such SNPs can make otherwise non-responding pseudoexons functional targets for SSO treatment and allow for inclusion or increased inclusion of the given pseudoexons in mature mRNA.

A549 cells were reverse transfected and LX-2 cells were forward transfected with nM SSO (5′-CAGACUACCAGACAUCUGACUAGAA-3′) (SEQ ID NO: 333) (targeting SEQ ID NO: 217; specific target sequence is underlined in SEQ ID NO: 217 in Table 9) using Lipofectamine RNAiMAX (Invitrogen). A non-targeting SSO (5′-GCUCAAUAUGCUACUGCCAUGCUUG-3′) (SEQ ID NO: 126) was used as a negative control. A549 cells were harvested 48 hours after transfection and LX-2 cells were harvested 24 hours after transfection using Trizol (Invitrogen). RNA was extracted using chloroform and isopropanol, complementary DNA (cDNA) was synthesized using the High-Capacity cDNA Reverse Transcription Kit (Applied Biosystems) and PCR was carried out using the TEMPase Hot Start DNA Polymerase (Ampliqon) with primers in LRRK2 exon 37 and 38-39. The PCR products were separated on a 1.5% SeaKem LE (Lonza) TBE agarose gel.

### Results

We identified a pseudoexon in intron 37 of the LRRK2 gene; chr12:40322690-40322887(+). The strength of the pseudoexon 5′ splice site is increased by a common SNP; rs10878372 A/G (frequency: A=0.77/G=0.23, 1000Genomes European population), that changes the MaxEnt score of −4.26 for the “A allele” to 4.84 for the “G allele”. This pseudoexon can be activated by an SSO targeting the Sweet Spot region only in gene alleles with the identified SNP (G allele). This enables G allele-specific activation of the pseudoexon in heterozygotes (=35% of the 1000Genomes European population) and activation of the pseudoexon from both alleles in individuals homozygous for the G allele, whereas the SSO will not activate the pseudoexon in individuals homozygous for the A allele.

Transfection of A549 and LX-2 cells with an SSO complementary to position +11 to +35 downstream of the 5′ splice site (SEQ ID NO: 217) resulted in increased inclusion of the LRRK2 pseudoexon in LX-2 cells that are heterozygous for the rs10878372 A/G SNP and did not affect inclusion of the pseudoexon in A549 cells that are homozygous for the A allele (compare FIGS. 10A and 10B). Inclusion of the LRRK2 pseudoexon will introduce 198 nt between exon 37 and 38 in the mature mRNA, including several in-frame premature translation termination codons.

### Conclusion

The activity and function of LRRK2 will be decreased by using an SSO targeting the Sweet Spot region to induce pseudoexon inclusion and thereby reduce the expression of LRRK2 gene alleles that harbor the identified SNP (G) for allele-specific targeting. Pseudoexon inclusion that introduces in-frame premature termination codons will reduce gene expression by degradation of the transcript or translation of a truncated and non-functional protein.

By the identification of a SNP that strengthens the 3′ or 5′ splice site of a pseudoexon, making the pseudoexon a functional target for SSO treatment to induce pseudoexon inclusion, it is possible to perform allele-specific targeting (or at least allele-preferred targeting), by screening for subjects that carry the specific SNP and dominant pathogenic mutation on the same allele before initiating treatment.
Reference: Alessi, D. R. & Sammler E (2018) Science, 360(6384):36-37. DOI: 10.1126/science.aar5683

### Example 16

### Aim of Study

In many autosomal dominant diseases, allele-specific down-regulation of expression from only the disease-causing (mutant) allele is preferred, because the mutant allele produces a protein with dominant negative effect that interferes with the normal protein produced from the wild type allele. Likewise, in diseases caused by gene duplication, where disease pathology is due to gene dosage sensitivity, allele specific down-regulation of only one allele is preferred. Activation of splicing of a pseudoexon that causes introduction of premature stop codons into the transcript from the mutant or overexpressed allele will reduce gene expression by degradation of the mutant transcript or translation of a truncated and non-functional protein.

SNPs that are located in the 23-mer 3′ splice site (−20 to +3 nt of the intron-exon border) or in the 9-mer 5′ splice site (−3 to +6 nt of the exon-intron border) affects the strength of the given splice site (changes the MaxEnt score). This can be exploited for allele-specific pseudoexon activation, since such high MaxEnt score SNP variants can make otherwise non-responding pseudoexons functional targets for SSO treatment and thereby allow for allele-specific inclusion or increased inclusion of the given pseudoexon in mature mRNA transcript. The principle of this invention is illustrated in FIG. 11.

It is therefore possible to perform allele-specific targeting (or at least allele-preferred targeting), by screening for subjects that carry the specific high MaxEnt score SNP variant before initiating treatment with SSOs that bind to the sweet spot.

### Materials and Methods

We used RNA-seq analysis to identify fragments with fully included pseudoexons in proprietary and publicly available RNA-seq data, and we used the dbSNP database (https://www.ncbi.nlm.nih.gov/SNP) to identify SNPs that strengthens the 3′ or 5′ splice sites (increases the MaxEnt scores). As illustrated in example 15, such high MaxEnt score SNP variants make otherwise non-responding pseudoexons functional targets for SSO treatment and allow for inclusion or increased inclusion of the given pseudoexons in mature mRNA.

### Results

We have identified the following additional examples of target pseudoexons where allele-specific activation by SSOs that bind in the sweet spot region is determined by SNP variants in splice site sequences (Table 9).

TABLE 9
Target pseudoexons where allele-specific
activation is determined by
SNP variants in splice site sequences.

| SSO | | | | |
|---|---|---|---|---|
| seq | | hg38 | | |
| ID | Gene | coordinates | SNP ID | Sweet Spot seq |
| 217 | LRRK2- | chr12: | rs10878372 | CTTTCTAGTCAGATGTCTGGTAGTCTGCAGC |
| | SNP | 40322690-40322887(+) | | |
| 218 | LMNB1 | chr5: | rs30485331 | CTGCGCTCAGCATGATATCCTGACTTACAGT |
| | | 126804263-126804357(+) | | |
| 219 | ATXN2- | chr12: | rs10849953 | GTTTCTGTTTCTCCACATCCTTGCTAACACT |
| | SNP | 111505733-111505834(−) | | |
| 220 | C3 | chr19: | rs11666563 | TCATACTCAAGTAGTTGCAAAAATACTTAAA |
| | | 6699217-6699330(−) | | |
| 221 | INPP5D | chr2: | rs28751960 | AACGAGCTTTGGGACTATCGTACCCCAAGAG |
| | | 233161943-233162017(+) | | |
| 222 | ELMO1 | chr7: | rs10226484 | TTTGTATTTTTAGCAGAGATGGGATTTCACC |
| | | 36884178-36884307(−) | | |
| 223 | SCN9A- | chr2: | rs6728078 | TAAAAAGTTGAGAAACAATAGATTCTGGTGA |
| | SNP | 166290605-166290765(−) | | |
| 224 | RBFOX1 | chr16: | rs9938245 | TGTGCTTTTTCTTATTTATATTTGCATTACA |
| | | 6876662-6876743(+) | | |
| 225 | SCN1A- | chr2: | rs151071889 | ATGTTAAGATGCTGGGTGTATGCTCATTTGT |
| | SNP | 166025123-166025259(−) | | |
| 226 | TRPM7 | chr15: | rs10775143 | TTGTGGTTTTTTTTAGTAGAGATGGGGTTTC |
| | | 50654856-50654997(−) | | |
| 227 | LINGO1 | chr15: | rs9920127 | CCAGCTTGCAAGCCTGCGGTTCAGTCTAGAA |
| | | 77628837-77628904(−) | | |
| 228 | RBFOX1- | chr16: | rs758497 | CTTGCACAGGAATCTATTCCAGTATCAGCTT |
| | SNP | 7707752-7707876(+) | | |
| 229 | GRIN2A | chr16: | rs7202848 | TGGCTCTAGTTCATCTGAGTCCAATTCTGAT |
| | | 9792916-9793121(−) | | |
| 230 | INPP5D- | chr2: | rs36145835 | CCAAGGTAAAGGCTGTGCCTCGGCCCTTGAA |
| | SNP | 233157160-233157272(+) | | |
| 231 | NIPA1 | chr15: | rs8026280 | ATATGTATATTTTAAAAGCAGTTCTGTTCTT |
| | | 22814387-22814505(+) | | |
| 232 | NIPA1- | chr15: | rs8026280 | ATATGTATATTTTAAAAGCAGTTCTGTTCTT |
| | 2 | 22814390-22814505(+) | | |
| 233 | SMARCA2 | chr9: | rs3793485 | TTTGAAGCCTCTGGTGCTTATCTTTCTTTTT |
| | | 2058740-2058881(+) | | |
| 234 | SMARCA2- | chr9: | rs7026165 | CTCCATTGTTTGACCCTAGTTCCAGCCGTGA |
| | 1 | 2187241-2187291(+) | | |
| 235 | CACNA1H- | chr16: | rs76364595 | CCCAGCGGGGCAGTGTGTGCAAATGCTGGGG |
| | SNP | 1216278-1216392(+) | | |
| 236 | GABRA4 | chr4: | rs2280073 | CATGCTTTTAACCTTTATAGAAAACCCAATC |
| | | 46965556-46965691(−) | | |
| 237 | RYR1 | chr19: | rs74493019 | CACCACGCCTGGCTAATTTTTGTATTTTTAG |
| | | 38495790-38495905(+) | | |
| 238 | RYR1- | chr19: | rs2915957 | ATGAGCACCAGCAAGCAATGTTTCCGTTATT |
| | 1 | 38506647-38506678(+) | | |
| 239 | GNE | chr9: | rs7850891 | TACAGAATAACAAATATTTCAAAATCCAAAA |
| | | 36235079-36235352(−) | | |
| 240 | TNNT2 | chr1: | rs149812965 | TCAGCATGTGGCATGTAGATATTAGGAGAAG |
| | | 201371183-201371324(−) | | |
| 241 | RUNX3 | chr1: | rs74060468 | AGGAGCCCTGCACGCTGTGCTCACGCCCACC |
| | | 24905894-24906104(−) | | |
| 242 | PDE3A | chr12: | rs10734706 | TACCATGTGCTAGTGTATGCATGTGCGTCTA |
| | | 20642899-20643080(+) | | |
| 243 | KIF21A | chr12: | rs58579369 | ATAATTGTATTTTTGATGGTTTAAATCAAGT |
| | | 39352972-39353016(−) | | |
| 244 | ABCA1 | chr9: | rs10991383 | CTTATCTTCTTTAGGATGGTAATGATCAGTT |
| | | 104827354-104827513(−) | | |
| 245 | RBFOX1- | chr16: | rs73488669 | TGAAGTCCATCTGCCGTTTCACGTATTTAGT |
| | SNP1 | 7596461-7596528(+) | | |
| 246 | INPP5D- | chr2: | rs62192895 | TATTCACTGTGCAGCTTCAGCCTGCAAATGG |
| | SNP1 | 233195220-233195283(+) | | |
| 247 | INPP5D- | chr2: | rs138305144 | TATTCACTGTGCAGCTTCAGCCTGCAAATGG |
| | SNP2 | 233195220-233195283(+) | | |
| 248 | SETX | chr9: | rs61421014 | CACCATACCTGGCTAATTTTTGTATTTTTAG |
| | | 132333254-132333350(−) | | |
| 249 | RYR2 | chr1: | rs74328949 | GGCTTTTTCATTGCTGCTTTAGGGACTGTAC |
| | | 237440251-237440408(+) | | |
| 250 | EPAS1 | chr2: | rs17035091 | GGCCTGAAAATAATTCGTGGTTTATGGAAAA |
| | | 46383277-46383409(+) | | |
| 251 | ADCY5 | chr3: | rs59340979 | TCCTCACACACAGTTAACAGGCCACTGATGT |
| | | 123350266-123350425(−) | | |
| 252 | INPP5- | chr2: | rs10179304 | TGTGTTGCGCTGTGACGTGATGACAGCTACA |
| | SNP3 | 233174264-233174308(+) | | |
| 253 | SPTLC1 | chr9: | rs56248784 | ATCACTTGAGCCCAGGAGTACAAGACCAGCC |
| | | 92033796-92033843(−) | | |
| 254 | GLUD1 | chr10: | rs6586053 | CTAAAGGAATGGAAAGTGAGCAAATAGGAGC |
| | | 87070016-87070105(−) | | |
| 255 | CHN1 | chr2: | rs6713884 | TCTATCATCTATGCCCATCCTACAGCTACAT |
| | | 174816132-174816212(−) | | |
| 256 | HTT- | chr4: | rs363141 | ACGGCGCCGCACAGTGGAGGCATCTGCTGCA |
| | SNP1 | 3160124-3160157(+) | | |
| 257 | HTT- | chr4: | rs59588638 | CCACCGTTGTGAAATCCGTGCCACCCGAGAG |
| | SNP2 | 3231898-3232034(+) | | |
| 258 | CACNA | chr3: | rs1380612 | CCTGGCATTGGCGTGAGGACACTGTTTCTTC |
| | 1D | 53746649-53746685(+) | | |
| 259 | SMARCA2- | chr9: | rs71329403 | CTCCATTGTTTGACCCTAGTTCCAGCCGTGA |
| | SNP1 | 2187241-2187291(+) | | |
| 260 | RYR1- | chr19: | rs2254484 | GTGACCCTCCGGCCCCTCTAGGACCCCTTCT |
| | SNP1 | 38526752-38526791(+) | | |
| 261 | CHRNA3 | chr15: | rs57005476 | CCGCTACCATGCCTAATTTTTTGTATTTTTA |
| | | 78599892-78600013(−) | | |
| 262 | LRRK2 | chr12: | rs11176195 | AAAATACATTGTCCTCATCCTTATGAAATTA |
| | | 40362410-40362491(+) | | |
| 263 | SNCA- | chr4: | rs2572320 | ATTAAACTTCTTTCTTTATAAAAAAAAAAAA |
| | SNP1 | 89791054-89791125(−) | | |
| 264 | VWF | chr12: | rs12827149 | GCAGTGTCAACTGCATTTCAAAGAACAGCTC |
| | | 6030332-6030439(−) | | |
| 265 | RET | chr10: | rs3026769 | TCTGATATGACCTTAGCTCTTTTCTCAAAGA |
| | | 43120411-43120519(+) | | |
| 266 | EIF2AK2 | chr2: | rs4648202 | ATAGTTTTATAAAGAAAAAAGTTGGCTGGGT |
| | | 37135964-37136142(−) | | |
| 267 | PDE3A- | chr12: | rs71446707 | TGTGGCTTTTACTCTGAGTAAGATGGGAAAT |
| | SNP1 | 20655553-20655674(+) | | |
| 268 | MYC | chr8: | rs5894898 | TGGCATCAATGTCCTATCCTGGGAAGTTGCA |
| | | 127739685-127739832(+) | | |
| 269 | LRRK2- | chr12: | rs10784536 | ATCTGATGCAATTTTCCTGCTTAAAACCCTT |
| | SNP2 | 40360475-40360605(+) | | |
| 270 | COL6A1- | chr21: | rs2277813 | CCCTGCCAGGGAGGGGCTGGGTGGGGAGGTG |
| | SNP1 | 45989272-45989324(+) | | |
| 271 | COL6A1- | chr21: | rs2277813 | CCCTGCCAGGGAGGGGCTGGGTGGGGAGGTG |
| | SNP2 | 45989310-45989324(+) | | |
| 272 | GNE- | chr9: | rs7850891 | TACAGAATAACAAATATTTCAAAATCCAAAA |
| | SNP1 | 36235079-36235096(−) | | |
| 273 | BPGM | chr7: | rs4728331 | CACCACACCTGGCTAATTTTTGTAATTTTTT |
| | | 134665899-134666020(+) | | |
| 274 | PTPRD | chr9: | rs10739160 | TGTTATGTTATATATATTGTTCTGCAATATA |
| | | 8351243-8351310(−) | | |
| 275 | EVI5 | chr1: | rs6665579 | CAGTGTATTTAGCTTCCCAGGTGTTTGTATC |
| | | 92711525-92711647(−) | | |
| 276 | HKDC1 | chr10: | rs58436137 | ATTAGAGCATTCCTTTGAGAGAGGTAGGGAG |
| | | 69238560-69238683(+) | | |
| 277 | KITLG | chr12: | rs11104938 | GTCAAACCAGACTGAATTTATAGAAATTATC |
| | | 88538470-88538514(−) | | |
| 278 | GNA11 | chr19: | rs1682801 | AGAGCCCCAGGTTGGAGGCCTGGGCCGTGAC |
| | | 3120650-3120739(+) | | |
| 279 | RAB5A | chr3: | rs4241539 | CTTTTATGACTTTGCTCCAGAAGTCACATAG |
| | | 19962022-19962119(+) | | |
| 280 | RPS6KB1 | chr17: | rs180520 | TTTTTTGGGGGTGGGTAAGGCACACGATTCA |
| | | 59937578-59937788(+) | | |
| 281 | PABPC1 | chr8: | rs3133580 | GTCATATTTTCTTGACTTTGGCCTCAGGAGA |
| | | 100714185-100714254(−) | | |
| 282 | PFKFB3 | chr10: | rs2148289 | GATTCTCCTGCCTCAGCCTCCCGAATAGCTG |
| | | 6218474-6218523(+) | | |
| 283 | HK1 | chr10: | rs10823359 | AAACAAAAGCAATTGTGCAAATACTGTGTTC |
| | | 69395846-69395909(+) | | |
| 284 | ADCY5- | chr3: | rs9856983 | CTTTCTTATGGATCCTAGTGTTTGCATGAGC |
| | SNP1 | 123386995-123387105(−) | | |
| 285 | GRIN2B | chr12: | rs7952915 | AGTTTTCAGCTTTCAACATTTAATTTAAGGT |
| | | 13879927-13880010(−) | | |
| 286 | MDM2 | chr12: | rs1201644 | ATAAGTGTTTTACAACCTACTTTCCATGAGA |
| | | 68820885-68820910(+) | | |
| 287 | RNF115- | chr1: | rs35545432 | AGATCGCACTACTGCACTCCAGCCTGGGTGA |
| | SNP1 | 145782938-145783156(−) | | |
| 288 | FLT1- | chr13: | rs880704 | AGATCTGTTGTAATCCCTGGGAATTTATTAC |
| | SNP11 | 28317744-28317857(−) | | |
| 289 | ATXN10 | chr22: | rs73889618 | TAGTGAACACTGCACCAATAACAGCATGTAT |
| | | 45797163-45797226(+) | | |
| 290 | DIAPH3- | chr13: | rs2210990 | TTATCTTTGTAAGCTCATTGCTTTATCTTCA |
| | SNP1 | 60161668-60161732(−) | | |
| 291 | TRPM7- | chr15: | rs3131596 | AATGGTAGAGTGGGCCAGGTGGCTTACGCCT |
| | SNP1 | 50585378-50585394(−) | | |
| 292 | ERBB4 | chr2: | rs3863853 | ATGTCTATCTTTTAATATTGTTTAAGATAAT |
| | | 211633762-211633784(−) | | |
| 293 | SREBF1 | chr17: | rs73297859 | TGTCAGAGATGGATCTGGGTTCTGTGGACAA |
| | | 17826979-17827055(−) | | |
| 294 | INSIG2 | chr2: | rs17047757 | TTTCAAAGTTTTATTGTTTGAAGATTCAGAC |
| | | 118103757-118103854(+) | | |

SNP ID. refers to SNP no. in dbSNP (https://www.ncbi.nlm.nih.gov/snp/).
Underlining indicates tested binding sequence of the SSO.
Binding is to the corresponding pre-mRNA.
hg38 Genome coordinates follow GFF/GTF format; one-based start and end coordinates.
Gene name has in certain cases been extended with a sequence code.

TABLE 10
SSOs able to perform allele specific activation of pseudoexons.

| SEQ | | | |
|---|---|---|---|
| ID | Gene | SSO name | SSO sequence 5′ -> 3′ |
| 295 | ABCA1 | ABCA1 SSO +11 | GAUCAUUACCAUCCUAAAGAAGAUA |
| 296 | ADCY5 | ADCY5 SSO1 | CAGUGGCCUGUUAACUGUGUGUGAG |
| | | +11 | |
| 297 | ADCY5 | ADCY5 SSO2 | AUGCAAACACUAGGAUCCAUAAGAA |
| | | +11 | |
| 298 | ATXN10 | ATXN10 SSO | AUGCUGUUAUUGGUGCAGUGUUCAC |
| | | +11 | |
| 299 | ATXN2 | ATXN2 SSO +11 | UUAGCAAGGAUGUGGAGAAACAGAA |
| 300 | BPGM | BPGM SSO +11 | AAUUACAAAAAUUAGCCAGGUGUGG |
| 301 | C3 | C3 SSO +11 | AGUAUUUUUGCAACUACUUGAGUAU |
| 302 | CACNA1D | CACNA1D SSO | AAACAGUGUCCUCACGCCAAUGCCA |
| | | +11 | |
| 303 | CACNA1H | CACNA1H SSO | AGCAUUUGCACACACUGCCCCGCUG |
| | | +11 | |
| 304 | CHN1 | CHN1 SSO +11 | AGCUGUAGGAUGGGCAUAGAUGAUA |
| 305 | CHRNA3 | CHRNA3 SSO | AAUACAAAAAAUUAGGCAUGGUAGC |
| | | +11 | |
| 306 | COL6A1 | COL6A1 SSO | UCCCCACCCAGCCCCUCCCUGGCAG |
| | | +11 | |
| 307 | DIAPH3 | DIAPH3 SSO | GAUAAAGCAAUGAGCUUACAAAGAU |
| | | +11 | |
| 308 | EIF2AK2 | EIF2AK2 SSO | AGCCAACUUUUUUCUUUAUAAAACU |
| | | +11 | |
| 309 | ELMO1 | ELMO1 SSO +11 | AAAUCCCAUCUCUGCUAAAAAUACA |
| 310 | EPAS1 | EPAS1 SSO +11 | CCAUAAACCACGAAUUAUUUUCAGG |
| 311 | ERBB4 | ERBB4 SSO +11 | UCUUAAACAAUAUUAAAAGAUAGAC |
| 312 | EVI5 | EVI5 SSO +11 | CAAACACCUGGGAAGCUAAAUACAC |
| 313 | FLT1 | FLT1 SSO +11 | UAAAUUCCCAGGGAUUACAACAGAU |
| 314 | GABRA4 | GABRA4 SSO | GGGUUUUCUAUAAAGGUUAAAAGCA |
| | | +11 | |
| 315 | GLUD1 | GLUD1 SSO +11 | CUAUUUGCUCACUUUCCAUUCCUUU |
| 316 | GNA11 | GNA11 SSO +11 | CGGCCCAGGCCUCCAACCUGGGGCU |
| 317 | GNE | GNE SSO +11 | GGAUUUUGAAAUAUUUGUUAUUCUG |
| 318 | GRIN2A | GRIN2A SSO | GAAUUGGACUCAGAUGAACUAGAGC |
| | | +11 | |
| 319 | GRIN2B | GRIN2B SSO | UAAAUUAAAUGUUGAAAGCUGAAAA |
| | | +11 | |
| 320 | HK1 | HK1 SSO +11 | ACAGUAUUUGCACAAUUGCUUUUGU |
| 321 | HKDC1 | HKDC1 SSO +11 | CUACCUCUCUCAAAGGAAUGCUCUA |
| 322 | HTT | HTT SSO1 +11 | GCAGAUGCCUCCACUGUGCGGCGCC |
| 323 | HTT | HTT SSO2 +11 | CGGGUGGCACGGAUUUCACAACGGU |
| 324 | INPP5D | INPP5D SSO1 | UGGGGUACGAUAGUCCCAAAGCUCG |
| | | +11 | |
| 325 | INPP5D | INPP5D SSO2 | AGGGCCGAGGCACAGCCUUUACCUU |
| | | +11 | |
| 326 | INPP5D | INPP5D SSO3 | UUGCAGGCUGAAGCUGCACAGUGAA |
| | | +11 | |
| 327 | INPP5D | INPP5D SSO4 | GCUGUCAUCACGUCACAGCGCAACA |
| | | +11 | |
| 328 | INSIG2 | INSIG2 SSO +11 | GAAUCUUCAAACAAUAAAACUUUGA |
| 329 | KIF21A | KIF21A SSO +11 | GAUUUAAACCAUCAAAAAUACAAUU |
| 330 | KITLG | KITLG SSO +11 | AUUUCUAUAAAUUCAGUCUGGUUUG |
| 331 | LINGO1 | LINGO1 SSO | AGACUGAACCGCAGGCUUGCAAGCU |
| | | +11 | |
| 332 | LMNB1 | LMNB1 SSO +11 | UAAGUCAGGAUAUCAUGCUGAGCGC |
| 333 | LRRK2 | LRRK2 SSO1 | CAGACUACCAGACAUCUGACUAGAA |
| | | +11 | |
| 334 | LRRK2 | LRRK2 SSO2 | UUCAUAAGGAUGAGGACAAUGUAUU |
| | | +11 | |
| 335 | LRRK2 | LRRK2 SSO3 | GUUUUAAGCAGGAAAAUUGCAUCAG |
| | | +11 | |
| 336 | MDM2 | MDM2 SSO +11 | AUGGAAAGUAGGUUGUAAAACACUU |
| 337 | MYC | MYC SSO +11 | ACUUCCCAGGAUAGGACAUUGAUGC |
| 338 | NIPA1 | NIPA1 SSO +11 | ACAGAACUGCUUUUAAAAUAUACAU |
| 339 | PABPC1 | PABPC1 SSO | CUGAGGCCAAAGUCAAGAAAAUAUG |
| | | +11 | |
| 340 | PDE3A | PDE3A SSO1 | CGCACAUGCAUACACUAGCACAUGG |
| | | +11 | |
| 341 | PDE3A | PDE3A SSO2 | CCCAUCUUACUCAGAGUAAAAGCCA |
| | | +11 | |
| 342 | PFKFB3 | PFKFB3 SSO +11 | UAUUCGGGAGGCUGAGGCAGGAGAA |
| 343 | PTPRD | PTPRD SSO +11 | UUGCAGAACAAUAUAUAUAACAUAA |
| 344 | RAB5A | RAB5A SSO +11 | GUGACUUCUGGAGCAAAGUCAUAAA |
| 345 | RBFOX1 | RBFOX1 SSO1 | AUGCAAAUAUAAAUAAGAAAAAGCA |
| | | +11 | |
| 346 | RBFOX1 | RBFOX1 SSO2 | UGAUACUGGAAUAGAUUCCUGUGCA |
| | | +11 | |
| 347 | RBFOX1 | RBFOX1 SSO3 | AAUACGUGAAACGGCAGAUGGACUU |
| | | +11 | |
| 348 | RET | RET SSO +11 | UGAGAAAAGAGCUAAGGUCAUAUCA |
| 349 | RNF115 | RNF115 SSO | CCAGGCUGGAGUGCAGUAGUGCGAU |
| | | +11 | |
| 350 | RPS6KB1 | RPS6KB1 SSO | UCGUGUGCCUUACCCACCCCCAAAA |
| | | +11 | |
| 351 | RUNX3 | RUNX3 SSO +11 | GGCGUGAGCACAGCGUGCAGGGCUC |
| 352 | RYR1 | RYR1 SSO1 +11 | AAAUACAAAAAUUAGCCAGGCGUGG |
| 353 | RYR1 | RYR1 SSO2 +11 | ACGGAAACAUUGCUUGCUGGUGCUC |
| 354 | RYR1 | RYR1 SSO3 +11 | GGGGUCCUAGAGGGGCCGGAGGGUC |
| 355 | RYR2 | RYR2 SSO +11 | AGUCCCUAAAGCAGCAAUGAAAAAG |
| 356 | SCN1A | SCN1A SSO +11 | AUGAGCAUACACCCAGCAUCUUAAC |
| 357 | SCN9A | SCN9A SSO +11 | CAGAAUCUAUUGUUUCUCAACUUUU |
| 358 | SETX | SETX SSO +11 | AAAUACAAAAAUUAGCCAGGUAUGG |
| 359 | SMARCA2 | SMARCA2 SSO1 | AGAAAGAUAAGCACCAGAGGCUUCA |
| | | +11 | |
| 360 | SMARCA2 | SMARCA2 SSO2 | GGCUGGAACUAGGGUCAAACAAUGG |
| | | +11 | |
| 361 | SNCA | SNCA SSO +11 | UUUUUUUUAUAAAGAAAGAAGUUUA |
| 362 | SPTLC1 | SPTLC1 SSO +11 | GGUCUUGUACUCCUGGGCUCAAGUG |
| 363 | SREBF1 | SREBF1 SSO | CCACAGAACCCAGAUCCAUCUCUGA |
| | | +11 | |
| 364 | TNNT2 | TNNT2 SSO +11 | UCCUAAUAUCUACAUGCCACAUGCU |
| 365 | TRPM7 | TRPM7 SSO1 | CCCCAUCUCUACUAAAAAAAACCAC |
| | | +11 | |
| 366 | TRPM7 | TRPM7 SSO2 | GUAAGCCACCUGGCCCACUCUACCA |
| | | +11 | |
| 367 | VWF | VWT SSO +11 | UGUUCUUUGAAAUGCAGUUGACACU |

### Selected Examples

### LMN1B:

Lamin B1 is encoded by the LMNB1 gene. Overexpression of lamin B1 causes progressive central nervous system demyelination, leading to autosomal dominant adult-onset demyelinating leukodystrophy (ADLD) (Giorgio et al. 2015; Giorgio et al. 2019). ADLD is an inherited, progressive and fatal disorder affecting myelin in the Central Nervous System (CNS). Allele-specific down regulation of one LMNB1 allele would be a suitable and promising therapeutic option for ADLD (Giorgio et al. 2019).

Further, expression of LMNB1 is increased in cancer and associated with cancer stage and patient prognosis. Down regulation of LMNB1 decreases proliferation and migration of cancer cells, and tumor growth. LMNB1 down regulation is therefore also a promising therapeutic strategy in cancers (Li et al. 2022). We identified a pseudoexon in intron 1 of the LMNB1 gene; chr5:126804263-126804357(+). The strength of the pseudoexon 5′ splice site is determined by a common SNP; rs30485331−−/insTT (frequency: −−=0.80/insTT=0.2, 1000Genomes European population). The insTT variant changes the MaxEnt score of 7.10 for the −− allele to −20.37 for the insTT allele (low MaxEnt score SNP variant). This pseudoexon can be activated by an SSO targeting the Sweet Spot region only in gene alleles without the identified SNP (insTT allele). This enables allele-specific activation of the pseudoexon in heterozygotes (=32% of the 1000Genomes European population) and activation of the pseudoexon from both alleles in individuals homozygous for the −− allele (high MaxEnt score SNP variant), whereas the SSO will not activate the pseudoexon in individuals homozygous for the insTT allele (low MaxEnt score SNP variant).

Inclusion of the chr5:126804263-126804357(+) LMNB1 pseudoexon will introduce 95 bp between exon 1 and exon 2 in the mRNA. This will introduce in-frame premature termination codons in the mRNA transcript. An LMNB1 mRNA transcript with inclusion of this pseudoexon is therefore predicted to be a target of nonsense-mediated decay, and increased pseudoexon inclusion will result in reduction of LMNB1 gene expression.

Sweet Spot for LMNB1 SSO targeting is shown in Table 9.

### REFERENCES

Giorgio E, Robyr D, Spielmann M, Ferrero E, Di Gregorio E, Imperiale D, Vaula G, Stamoulis G, Santoni F, Atzori C, Gasparini L, Ferrera D, Canale C, Guipponi M, Pennacchio L A, Antonarakis S E, Brussino A, Brusco A. A large genomic deletion leads to enhancer adoption by the lamin B1 gene: a second path to autosomal dominant adult-onset demyelinating leukodystrophy (ADLD). Hum Mol Genet. 2015 Jun. 1; 24(11):3143-54. doi: 10.1093/hmg/ddv065. Epub 2015 Feb. 20. PMID: 25701871; PMCID: PMC4424952.
Giorgio E, Lorenzati M, Rivetti di Val Cervo P, Brussino A, Cernigoj M, Della Sala E, Bartoletti Stella A, Ferrero M, Caiazzo M, Capellari S, Cortelli P, Conti L, Cattaneo E, Buffo A, Brusco A. Allele-specific silencing as treatment for gene duplication disorders: proof-of-principle in autosomal dominant leukodystrophy. Brain. 2019 Jul. 1; 142(7):1905-1920. doi: 10.1093/brain/awz139. PMID: 31143934.
Li J, Sun Z, Cui Y, Qin L, Wu F, Li Y, Du N, Li X. Knockdown of LMNB1 Inhibits the Proliferation of Lung Adenocarcinoma Cells by Inducing DNA Damage and Cell Senescence. Front Oncol. 2022 May 31; 12:913740. doi: 10.3389/fonc.2022.913740. PMID: 35712471; PMCID: PMC9194513.

### ATXN2:

Ataxin-2 is encoded by the ATXN2 gene. Spinocerebellar ataxia 2 (SCA2) is an autosomal dominant lethal disease caused by expansion to more than 32 CAG repeats in one ATXN2 allele (Laffita-Mesa et al. 2021). Alleles with intermediate (≥29CAG/CAA repeats) expansions in ATXN2 increase the risk for many other neurological diseases. Lowering ATXN2 expression in Amyotrophic lateral sclerosis (ALS) mice prolongs their survival, suggesting that lowering of ATXN2 disease allele expression could be therapeutically relevant for both ALS and SCA2 (Laffita-Mesa et al. 2021).

Allele-specific down-regulation of one ATXN2 disease-associated allele would therefore be a suitable therapeutic option for SCA2, ALS and other neurodegenerative diseases.

We identified a pseudoexon in intron 14 of the ATXN2 gene; chr12:111505733-111505834(−). The strength of the pseudoexon 5′ splice site is determined by a common SNP; rs10849953 A/G (frequency: A=0.58/G=0.42, 1000Genomes Japanese population). The G variant changes the MaxEnt score of −1.34 for the A allele to 6.84 for the G allele (high MaxEnt score SNP variant). Therefore, this pseudoexon can be activated by an SSO targeting the Sweet Spot region only in gene alleles with the identified SNP (G allele). This enables allele-specific activation of the pseudoexon in heterozygotes (=49% of the 1000Genomes Japanese population) and activation of the pseudoexon from both alleles in individuals homozygous for the G allele, whereas the SSO will not activate the pseudoexon in individuals homozygous for the A allele (low MaxEnt score SNP variant).

Inclusion of the chr12:111505733-111505834(−)

ATXN2 pseudoexon will introduce 102 bp between exon 14 and exon 15 in the mRNA. This will introduce an in-frame premature termination codon in the mRNA transcript. An ATXN2 mRNA transcript with inclusion of this pseudoexon is therefore predicted to be a target of nonsense-mediated decay, and increased pseudoexon inclusion will result in reduction of ATXN2 gene expression. Sweet Spot for ATXN2 SSO targeting is shown in Table 9.
Reference: Laffita-Mesa J M, Paucar M, Svenningsson P. Ataxin-2 gene: a powerful modulator of neurological disorders. Curr Opin Neurol. 2021 Aug. 1; 34(4):578-588. doi: 10.1097/WCO.0000000000000959. PMID: 34010218; PMCID: PMC8279897.

1.-42. (canceled)

43. A composition comprising a splice switching oligonucleotide (SSO), said composition comprising
an SSO complementary or substantially complementary to region within a nucleic acid selected from the group consisting of
a nucleic acid according to any of SEQ ID NO's: 217-294;
a nucleic acid comprising 1 or 2 or 3 substitutions when compared to any of SEQ ID NO's: 217-294; or
a nucleic acid sequence having at least 90% sequence identity to any of SEQ ID NO's: 217-294;
or
an SSO selected from the group consisting of:
a nucleic acid according to any of SEQ ID NO's: 295-367; or
a nucleic acid comprising 1 or 2 or 3 substitutions when compared to any of SEQ ID NO's: 295-367; or
a nucleic acid sequence having at least 90% sequence identity to any of SEQ ID NO's: 295-367;
wherein said SSO being complementary or substantially complementary to a target pre-mRNA, said target pre-mRNA comprising:
a function-disabling pseudoexon comprising:
at the 5′-end a 3′ splice site; and
at the 3′-end a 5′ splice site;
wherein said SSO is complementary or substantially complementary to the target pre-RNA at a region +9 to +39 downstream to the 5′ splice site of said pseudoexon.

44. The composition for use according to claim 43, wherein the SSO is complementary or substantially complementary to region within a nucleic acid selected from the group consisting of
a nucleic acid according to any of SEQ ID NO's: 217-294;
a nucleic acid comprising 1 or 2 or 3 substitutions when compared to any of SEQ ID NO's: 217-294; or
a nucleic acid sequence having at least 90% sequence identity to any of SEQ ID NO's: 217-294;

45. The composition for use according to claim 43, wherein the SSO is complementary or substantially complementary to region within a nucleic acid selected from the group consisting of
a nucleic acid according to any of SEQ ID NO: 217;
a nucleic acid comprising 1 or 2 or 3 substitutions when compared to any of SEQ ID NO's: 217; or
a nucleic acid sequence having at least 90% sequence identity to any of SEQ ID NO's: 217.

46. The composition according to claim 43, wherein said SSO comprises a sequence, which is substantially complementary to the polynucleotide in the pre-mRNA, and comprises at the most 3 mismatches.

47. The composition according to claim 43, wherein said SSO comprises one or more artificial nucleotides.

48. The composition according to claim 43, wherein said SSO has a length in the range 9-100 nucleotides.

49. The composition according to claim 43, wherein said SSO has a length in the range 9-31 nucleotides.

50. The composition for use according to claim 43, wherein the oligonucleotide does not mediate RNAse H mediated degradation of the mRNA.

51. A method for treating or alleviating a disease in a subject, the method comprising administrating to a subject in need thereof, a composition comprising a splice switching oligonucleotide (SSO), said composition comprising
an SSO complementary or substantially complementary to region within a nucleic acid selected from the group consisting of
a nucleic acid according to any of SEQ ID NO's: 217-294;
a nucleic acid comprising 1 or 2 or 3 substitutions when compared to any of SEQ ID NO's: 217-294; or
a nucleic acid sequence having at least 90% sequence identity to any of SEQ ID NO's: 217-294;
or
an SSO selected from the group consisting of:
a nucleic acid according to any of SEQ ID NO's: 295-367; or
a nucleic acid comprising 1 or 2 or 3 substitutions when compared to any of SEQ ID NO's: 295-367; or
a nucleic acid sequence having at least 90% sequence identity to any of SEQ ID NO's: 295-367;
wherein said SSO being complementary or substantially complementary to a target pre-mRNA, said target pre-mRNA comprising:
a function-disabling pseudoexon comprising:
at the 5′-end a 3′ splice site; and
at the 3′-end a 5′ splice site;
wherein said SSO is complementary or substantially complementary to the target pre-RNA at a region +9 to +39 downstream to the 5′ splice site of said pseudoexon.

52. The method according to claim 51, wherein the disease is selected from the group consisting of cancer, inflammatory diseases, Neurodegenerative or neurological diseases, Metabolic conditions, Chronic liver disease and Inherited retinal dystrophies (IRDs).

53. The method according to claim 51, wherein the composition is administered to a subject who is heterozygous for a sequence variation (SNP) in the pre-mRNA targeted by the SSO, whereby the SSO promotes inclusion of a function-disabling pseudoexon to a greater extent in a disease-causing allele compared to the other allele;
or
wherein the composition is administered to a subject who is homozygous for the SNP variation (SNP) in the pre-mRNA targeted by the SSO.

54. The method according to claim 51, wherein the subject is heterozygous for a sequence variation (SNP) in the 5′ splice site and/or the 3′ splice site of the function-disabling pseudoexon.

55. The method according to claim 51, wherein the subject is heterozygous for a sequence variation (SNP) in the 5′ splice site and/or the 3′ splice site of the function-disabling pseudoexon, and
wherein the SNP is a point mutation, an insertion of 1-20 nucleotides at the SNP position or a deletion of 1-20 nucleotides at the SNP position.

56. The method according to claim 51, wherein the subject is heterozygous for a sequence variation (SNP) in the 5′ splice site and/or the 3′ splice site of the function-disabling pseudoexon, and
wherein the heterozygosity in the disease-causing gene, increases inclusion of the pseudoexon into the mature mRNA to a larger extent from one allele than in the corresponding gene on the other allele, when brought in contact with the SSO.

57. The method according to claim 51, wherein the subject harbors a SNP in the disease-causing allele selected from the group of SNP IDs according to Table 9.

58. The method according to claim 51, wherein the pre-mRNA encodes for LRRK2.

59. The method according to claim 51, wherein the pre-mRNA encodes for LRRK2 and the subject is heterozygous for a disease causing mutation in LRRK2.


SEQUENCE LISTING (369 sequences)

SEQ ID NO: 1 - DNA, 31 bp, Homo sapiens
  source 1..31: mol_type = genomic DNA
aagtttataggttaaatatttattaaagcca

SEQ ID NO: 2 - DNA, 31 bp, Homo sapiens
  source 1..31: mol_type = genomic DNA
attgcacagccttcttcaaggaggagtcccc

SEQ ID NO: 3 - DNA, 31 bp, Homo sapiens
  source 1..31: mol_type = genomic DNA
tgaactgtgtagtgcctcagaaaatatcaag

SEQ ID NO: 4 - DNA, 31 bp, Homo sapiens
  source 1..31: mol_type = genomic DNA
aatccagcctacagccaccaaactgggtggg

SEQ ID NO: 5 - DNA, 31 bp, Homo sapiens
  source 1..31: mol_type = genomic DNA
atggcacatgtggagaatcttccatggtgtg

SEQ ID NO: 6 - DNA, 31 bp, Homo sapiens
  source 1..31: mol_type = genomic DNA
cagctctggggtgcctgcccccatgggggaa

SEQ ID NO: 7 - DNA, 31 bp, Homo sapiens
  source 1..31: mol_type = genomic DNA
caggaagggagtgtggaattacagccttcta

SEQ ID NO: 8 - DNA, 31 bp, Homo sapiens
  source 1..31: mol_type = genomic DNA
gtgcacctgccagggttgaatctgtagggct

SEQ ID NO: 9 - DNA, 31 bp, Homo sapiens
  source 1..31: mol_type = genomic DNA
caggcagcggcagtgtagcttttccactgtc

SEQ ID NO: 10 - DNA, 31 bp, Homo sapiens
  source 1..31: mol_type = genomic DNA
acatgaaagcacatgagctgcttaatgttgg

SEQ ID NO: 11 - DNA, 31 bp, Homo sapiens
  source 1..31: mol_type = genomic DNA
cggaactgggatttggacctaccttcagaac

SEQ ID NO: 12 - DNA, 31 bp, Homo sapiens
  source 1..31: mol_type = genomic DNA
aattcaagcttagtttatgaaggactgaaca

SEQ ID NO: 13 - DNA, 31 bp, Homo sapiens
  source 1..31: mol_type = genomic DNA
gagctcagctcctaaggatgtgcaggggcag

SEQ ID NO: 14 - DNA, 31 bp, Homo sapiens
  source 1..31: mol_type = genomic DNA
ggggcaccaaagtcttccctgtcccatcccc

SEQ ID NO: 15 - DNA, 31 bp, Homo sapiens
  source 1..31: mol_type = genomic DNA
ggatagatagagaggatccagaggctaaacg

SEQ ID NO: 16 - DNA, 31 bp, Homo sapiens
  source 1..31: mol_type = genomic DNA
caggatattggaagtcaggtcccatcttcgt

SEQ ID NO: 17 - DNA, 31 bp, Homo sapiens
  source 1..31: mol_type = genomic DNA
ttgactattggcctattttcctactgggcaa

SEQ ID NO: 18 - DNA, 31 bp, Homo sapiens
  source 1..31: mol_type = genomic DNA
caaaagccatctttatgagcactcaggtgct

SEQ ID NO: 19 - DNA, 31 bp, Homo sapiens
  source 1..31: mol_type = genomic DNA
gtgaactgacatttgcttaagccccgtgtca

SEQ ID NO: 20 - DNA, 31 bp, Homo sapiens
  source 1..31: mol_type = genomic DNA
ctggtgtactaagaatacagccacattcccc

SEQ ID NO: 21 - DNA, 31 bp, Homo sapiens
  source 1..31: mol_type = genomic DNA
gtcttagttgttgaagctaagcgagatgcag

SEQ ID NO: 22 - DNA, 31 bp, Homo sapiens
  source 1..31: mol_type = genomic DNA
acagtgtagggagttaaaaagtgccatgaat

SEQ ID NO: 23 - DNA, 31 bp, Homo sapiens
  source 1..31: mol_type = genomic DNA
acggaaaagcgcctttccctcagttgttcct

SEQ ID NO: 24 - DNA, 31 bp, Homo sapiens
  source 1..31: mol_type = genomic DNA
tctttagtatatgcagaagtcagaaagtcag

SEQ ID NO: 25 - DNA, 31 bp, Homo sapiens
  source 1..31: mol_type = genomic DNA
agcacctactctcagtccactacattgagtc

SEQ ID NO: 26 - DNA, 31 bp, Homo sapiens
  source 1..31: mol_type = genomic DNA
tgctccctgcaagtactaggaatgcaggctg

SEQ ID NO: 27 - DNA, 31 bp, Homo sapiens
  source 1..31: mol_type = genomic DNA
taagtttaccatcttatatggacgtggtttg

SEQ ID NO: 28 - DNA, 31 bp, Homo sapiens
  source 1..31: mol_type = genomic DNA
tggatttttggtagagacggggttttgccat

SEQ ID NO: 29 - DNA, 31 bp, Homo sapiens
  source 1..31: mol_type = genomic DNA
acagaccactgaaccaagaaccaagagagaa

SEQ ID NO: 30 - DNA, 31 bp, Homo sapiens
  source 1..31: mol_type = genomic DNA
ccttttcacattggcgtctctcacttagcga

SEQ ID NO: 31 - DNA, 31 bp, Homo sapiens
  source 1..31: mol_type = genomic DNA
catctatatgttcaacttacattttattttc

SEQ ID NO: 32 - DNA, 31 bp, Homo sapiens
  source 1..31: mol_type = genomic DNA
cactgtgcctggccggttttgtcttctaagt

SEQ ID NO: 33 - DNA, 31 bp, Homo sapiens
  source 1..31: mol_type = genomic DNA
tggcttcctaccccatttatttatacttcac

SEQ ID NO: 34 - DNA, 31 bp, Homo sapiens
  source 1..31: mol_type = genomic DNA
ttcagatcttttgagctagaacaaaaaaaca

SEQ ID NO: 35 - DNA, 31 bp, Homo sapiens
  source 1..31: mol_type = genomic DNA
tgttaaatctgatgttaatgattatttcatc

SEQ ID NO: 36 - DNA, 31 bp, Homo sapiens
  source 1..31: mol_type = genomic DNA
attatagagtttaatcttattttgagggcct

SEQ ID NO: 37 - DNA, 31 bp, Homo sapiens
  source 1..31: mol_type = genomic DNA
tcctcagcctgtgttttagctttctaaatgt

SEQ ID NO: 38 - DNA, 31 bp, Homo sapiens
  source 1..31: mol_type = genomic DNA
cagcatgtggggagaggcgcgtcactgggag

SEQ ID NO: 39 - DNA, 31 bp, Homo sapiens
  source 1..31: mol_type = genomic DNA
gcaggggcctcggcccaggggctccgacctc

SEQ ID NO: 40 - DNA, 31 bp, Homo sapiens
  source 1..31: mol_type = genomic DNA
tttcataggtaggaatatttaggatatttgg

SEQ ID NO: 41 - DNA, 31 bp, Homo sapiens
  source 1..31: mol_type = genomic DNA
cactgtgtgtagccttcttctttctctatgg

SEQ ID NO: 42 - DNA, 31 bp, Homo sapiens
  source 1..31: mol_type = genomic DNA
agttagctaatcattttctctgtttatattt

SEQ ID NO: 43 - DNA, 31 bp, Homo sapiens
  source 1..31: mol_type = genomic DNA
gtaagactgacatgccttctaccctccagaa

SEQ ID NO: 44 - DNA, 31 bp, Homo sapiens
  source 1..31: mol_type = genomic DNA
accctctagtataatatatgccacattaaaa

SEQ ID NO: 45 - DNA, 31 bp, Homo sapiens
  source 1..31: mol_type = genomic DNA
caccatgcccaactaacttttctattttttg

SEQ ID NO: 46 - DNA, 31 bp, Homo sapiens
  source 1..31: mol_type = genomic DNA
tactgctaagtggcatgttttgttttatgct

SEQ ID NO: 47 - DNA, 31 bp, Homo sapiens
  source 1..31: mol_type = genomic DNA
gactggcctggggtgtgggaatctagggcct

SEQ ID NO: 48 - DNA, 31 bp, Homo sapiens
  source 1..31: mol_type = genomic DNA
cccccattgagagctgtgtcttcaaactctt

SEQ ID NO: 49 - DNA, 31 bp, Homo sapiens
  source 1..31: mol_type = genomic DNA
gctttgtggcagagaggggactggcactttg

SEQ ID NO: 50 - DNA, 31 bp, Homo sapiens
  source 1..31: mol_type = genomic DNA
tattgatcagaacccttgtttcagataacat

SEQ ID NO: 51 - DNA, 31 bp, Homo sapiens
  source 1..31: mol_type = genomic DNA
catttggtattacaccaggttcctttaggca

SEQ ID NO: 52 - DNA, 31 bp, Homo sapiens
  source 1..31: mol_type = genomic DNA
tcttctggctgggacatgggatatatcctgt

SEQ ID NO: 53 - DNA, 31 bp, Homo sapiens
  source 1..31: mol_type = genomic DNA
acatatgtctatgttcagtgccaatagttaa

SEQ ID NO: 54 - DNA, 31 bp, Homo sapiens
  source 1..31: mol_type = genomic DNA
atgtagatgcctttggttttgttttgttttc

SEQ ID NO: 55 - DNA, 31 bp, Homo sapiens
  source 1..31: mol_type = genomic DNA
ccgcctgccgcacagcgtcgtttgcaaaacc

SEQ ID NO: 56 - DNA, 31 bp, Homo sapiens
  source 1..31: mol_type = genomic DNA
agatgtgaacaggtccctcttctttgggctt

SEQ ID NO: 57 - DNA, 31 bp, Homo sapiens
  source 1..31: mol_type = genomic DNA
cctgttaggaaccgggaggcacagcaggagg

SEQ ID NO: 58 - DNA, 31 bp, Homo sapiens
  source 1..31: mol_type = genomic DNA
atttttagttatttgagaaatctccacgttt

SEQ ID NO: 59 - DNA, 31 bp, Homo sapiens
  source 1..31: mol_type = genomic DNA
atatttatgtttttaagaaattaccaagctg

SEQ ID NO: 60 - DNA, 31 bp, Homo sapiens
  source 1..31: mol_type = genomic DNA
catgaattttttggtttcccattgcatataa

SEQ ID NO: 61 - DNA, 31 bp, Homo sapiens
  source 1..31: mol_type = genomic DNA
tatttttagttactttttatttttgagacct

SEQ ID NO: 62 - DNA, 31 bp, Homo sapiens
  source 1..31: mol_type = genomic DNA
actcgctgaagtgggggcaggttaagaagcc

SEQ ID NO: 63 - DNA, 31 bp, Homo sapiens
  source 1..31: mol_type = genomic DNA
gcagccaagactctgttcaagtttgtgtggg

SEQ ID NO: 64 - DNA, 31 bp, Homo sapiens
  source 1..31: mol_type = genomic DNA
taaatagtctcaaaggtggaggaggccccag

SEQ ID NO: 65 - DNA, 31 bp, Homo sapiens
  source 1..31: mol_type = genomic DNA
cactgtgcccgggtttttgtaccccatataa

SEQ ID NO: 66 - DNA, 31 bp, Homo sapiens
  source 1..31: mol_type = genomic DNA
ttaccccttttgctacctttaatccttgcac

SEQ ID NO: 67 - DNA, 31 bp, Homo sapiens
  source 1..31: mol_type = genomic DNA
caagatcatggggagatgaaagtagcatcaa

SEQ ID NO: 68 - DNA, 31 bp, Homo sapiens
  source 1..31: mol_type = genomic DNA
ctgatattgatgtgaaaaattgatattttgg

SEQ ID NO: 69 - DNA, 31 bp, Homo sapiens
  source 1..31: mol_type = genomic DNA
cccaccttagctactctcaagcagctgagac

SEQ ID NO: 70 - DNA, 31 bp, Homo sapiens
  source 1..31: mol_type = genomic DNA
cactgttgggttgtttttcttttgaagtgtt

SEQ ID NO: 71 - DNA, 31 bp, Homo sapiens
  source 1..31: mol_type = genomic DNA
aggcatgctggacaaatggattcacatgtgc

SEQ ID NO: 72 - DNA, 31 bp, Homo sapiens
  source 1..31: mol_type = genomic DNA
tcattaaatggtgcatccggatcagaaccta

SEQ ID NO: 73 - DNA, 31 bp, Homo sapiens
  source 1..31: mol_type = genomic DNA
ccaacttttctctcacataaaatctgtctgc

SEQ ID NO: 74 - DNA, 31 bp, Homo sapiens
  source 1..31: mol_type = genomic DNA
gggttaacaagtgctggcgcggggtccgcta

SEQ ID NO: 75 - DNA, 31 bp, Homo sapiens
  source 1..31: mol_type = genomic DNA
tctctgggtcagtactttccgggtgggagag

SEQ ID NO: 76 - DNA, 31 bp, Homo sapiens
  source 1..31: mol_type = genomic DNA
atcatggggcaatttcacccattctgttctc

SEQ ID NO: 77 - DNA, 31 bp, Homo sapiens
  source 1..31: mol_type = genomic DNA
taaactgtgcactgttctatgtaatatgatt

SEQ ID NO: 78 - DNA, 31 bp, Homo sapiens
  source 1..31: mol_type = genomic DNA
gatccccatatacaccttcaacaattatcta

SEQ ID NO: 79 - DNA, 31 bp, Homo sapiens
  source 1..31: mol_type = genomic DNA
ctgtgaaggccgtgaggtaagagaccttgac

SEQ ID NO: 80 - DNA, 31 bp, Homo sapiens
  source 1..31: mol_type = genomic DNA
ggaatgggggaaatggagaataaccagtata

SEQ ID NO: 81 - DNA, 31 bp, Homo sapiens
  source 1..31: mol_type = genomic DNA
aagggcggtagacatgaactgaagtcacgtt

SEQ ID NO: 82 - DNA, 31 bp, Homo sapiens
  source 1..31: mol_type = genomic DNA
ggtagatttaagggcggtagacatgaactga

SEQ ID NO: 83 - DNA, 31 bp, Homo sapiens
  source 1..31: mol_type = genomic DNA
cgtgtagcaagttgcagtctggggacttggt

SEQ ID NO: 84 - DNA, 31 bp, Homo sapiens
  source 1..31: mol_type = genomic DNA
tgaaccctgggtagcaactcttgagcgaaga

SEQ ID NO: 85 - DNA, 31 bp, Homo sapiens
  source 1..31: mol_type = genomic DNA
tagattagacatcagtggtattgaaatttaa

SEQ ID NO: 86 - DNA, 31 bp, Homo sapiens
  source 1..31: mol_type = genomic DNA
tgctcagagactggatccttttaagagtggg

SEQ ID NO: 87 - DNA, 31 bp, Homo sapiens
  source 1..31: mol_type = genomic DNA
cttatagagtgaagggtccctaggccaagac

SEQ ID NO: 88 - DNA, 31 bp, Homo sapiens
  source 1..31: mol_type = genomic DNA
ttggaggagggagaagcccaccttttaagga

SEQ ID NO: 89 - DNA, 31 bp, Homo sapiens; identical to SEQ ID NO: 88
  source 1..31: mol_type = genomic DNA

SEQ ID NO: 90 - DNA, 31 bp, Homo sapiens
  source 1..31: mol_type = genomic DNA
cagtgccatttaggatgtactgcataagttt

SEQ ID NO: 91 - DNA, 31 bp, Homo sapiens; identical to SEQ ID NO: 90
  source 1..31: mol_type = genomic DNA

SEQ ID NO: 92 - DNA, 31 bp, Homo sapiens
  source 1..31: mol_type = genomic DNA
tggagagttagggttaggatgttagaattgc

SEQ ID NO: 93 - DNA, 31 bp, Homo sapiens
  source 1..31: mol_type = genomic DNA
tagcattaacagtataggtagggaatctggt

SEQ ID NO: 94 - DNA, 31 bp, Homo sapiens; identical to SEQ ID NO: 93
  source 1..31: mol_type = genomic DNA

SEQ ID NO: 95 - DNA, 31 bp, Homo sapiens
  source 1..31: mol_type = genomic DNA
caaggctacattgggagacagtgaaaggcag

SEQ ID NO: 96 - DNA, 31 bp, Homo sapiens
  source 1..31: mol_type = genomic DNA
ccagaagcaggatttgcagggtagggttatg

SEQ ID NO: 97 - DNA, 31 bp, Homo sapiens
  source 1..31: mol_type = genomic DNA
cactgtgcccaaccttggagtgagatgaata

SEQ ID NO: 98 - DNA, 31 bp, Homo sapiens
  source 1..31: mol_type = genomic DNA
ggagtaataaatgtctgtcaagggcagcatc

SEQ ID NO: 99 - DNA, 31 bp, Homo sapiens
  source 1..31: mol_type = genomic DNA
tcaatggatgtgctagccgctccatttggct

SEQ ID NO: 100 - DNA, 31 bp, Homo sapiens
  source 1..31: mol_type = genomic DNA
aatatctgattgtcggatttatcccaggaag

SEQ ID NO: 101 - DNA, 31 bp, Homo sapiens
  source 1..31: mol_type = genomic DNA
ggcttcagagatgaagagagtatttctcctc

SEQ ID NO: 102 - DNA, 31 bp, Homo sapiens
  source 1..31: mol_type = genomic DNA
atcaggaagcttatggtagaaggctaagggg

SEQ ID NO: 103 - DNA, 31 bp, Homo sapiens
  source 1..31: mol_type = genomic DNA
gctatgtcagtaggcaactggagcacagtcc

SEQ ID NO: 104 - DNA, 31 bp, Homo sapiens
  source 1..31: mol_type = genomic DNA
ttggcagagtttagcatccaggcagggtgaa

SEQ ID NO: 105 - DNA, 31 bp, Homo sapiens
  source 1..31: mol_type = genomic DNA
tcagtaaagtttaaagggtagctaggagcca

SEQ ID NO: 106 - DNA, 31 bp, Homo sapiens
  source 1..31: mol_type = genomic DNA
caggccaggataagttgtcgaagtcacaatg

SEQ ID NO: 107 - DNA, 31 bp, Homo sapiens
  source 1..31: mol_type = genomic DNA
gtgcttcagggatgaatccagaggttaacta

SEQ ID NO: 108 - DNA, 31 bp, Homo sapiens
  source 1..31: mol_type = genomic DNA
ctgtgtgcccacttaaactgttttgataatg

SEQ ID NO: 109 - DNA, 31 bp, Homo sapiens
  source 1..31: mol_type = genomic DNA
gaactcaagtagaccctagtcagcttcaagt

SEQ ID NO: 110 - DNA, 31 bp, Homo sapiens
  source 1..31: mol_type = genomic DNA
tgtcatccccactttacagagaagtgggtta

SEQ ID NO: 111 - DNA, 31 bp, Homo sapiens
  source 1..31: mol_type = genomic DNA
gccttttccaaaccaaagaacaaagcgggag

SEQ ID NO: 112 - DNA, 31 bp, Homo sapiens
  source 1..31: mol_type = genomic DNA
tttaggccaaagcaggaataagatgtggaca

SEQ ID NO: 113 - DNA, 31 bp, Homo sapiens
  source 1..31: mol_type = genomic DNA
agtgactcccaaagatcagggtcctgtttgg

SEQ ID NO: 114 - DNA, 31 bp, Homo sapiens
  source 1..31: mol_type = genomic DNA
aggactataggctgcgcatagctcgaaggag

SEQ ID NO: 115 - DNA, 31 bp, Homo sapiens
  source 1..31: mol_type = genomic DNA
aaactccaggtgtctgtaataagggacaggg

SEQ ID NO: 116 - DNA, 31 bp, Homo sapiens
  source 1..31: mol_type = genomic DNA
cctgaggttgggagtttgagagcagcctgaa

SEQ ID NO: 117 - DNA, 31 bp, Homo sapiens
  source 1..31: mol_type = genomic DNA
gtagggagaaaaaaaaaagcctgtgctgtaa

SEQ ID NO: 118 - DNA, 31 bp, Homo sapiens
  source 1..31: mol_type = genomic DNA
gatgaacacaacacagtctttgctgaaggag

SEQ ID NO: 119 - DNA, 31 bp, Homo sapiens
  source 1..31: mol_type = genomic DNA
ggagtttagagtagtggattttatggactcg

SEQ ID NO: 120 - DNA, 31 bp, Homo sapiens
  source 1..31: mol_type = genomic DNA
gagggagaaaaacttttatcttggggctact

SEQ ID NO: 121 - DNA, 31 bp, Homo sapiens
  source 1..31: mol_type = genomic DNA
aatctgtattctctagtcaaggagggatggg

SEQ ID NO: 122 - DNA, 31 bp, Homo sapiens
  source 1..31: mol_type = genomic DNA
actagtagatcacacccctagatagtttagc

SEQ ID NO: 123 - DNA, 31 bp, Homo sapiens
  source 1..31: mol_type = genomic DNA
aattggagcaaatgcctgtagcttctgtcag

SEQ ID NO: 124 - DNA, 31 bp, Homo sapiens
  source 1..31: mol_type = genomic DNA
ttacagtccgttgtaaatgagtgacctagcg

SEQ ID NO: 125 - DNA, 31 bp, Homo sapiens
  source 1..31: mol_type = genomic DNA
gccggggctctgtagtgggttttgtgcccca

SEQ ID NO: 126 - RNA, 25 nt, synthetic construct
  misc_feature 1..25: note = -
  source 1..25: mol_type = other RNA
gctcaatatgctactgccatgcttg

SEQ ID NO: 127 - RNA, 25 nt, synthetic construct
  misc_feature 1..25: note = -
  source 1..25: mol_type = other RNA
gtccttcataaactaagcttgaatt

SEQ ID NO: 128 - RNA, 25 nt, synthetic construct
  misc_feature 1..25: note = -
  source 1..25: mol_type = other RNA
agtccttcataaactaagcttgaat

SEQ ID NO: 129 - RNA, 25 nt, synthetic construct
  misc_feature 1..25: note = -
  source 1..25: mol_type = other RNA
cagtccttcataaactaagcttgaa

SEQ ID NO: 130 - RNA, 25 nt, synthetic construct
  misc_feature 1..25: note = -
  source 1..25: mol_type = other RNA
tcagtccttcataaactaagcttga

SEQ ID NO: 131 - RNA, 25 nt, synthetic construct
  misc_feature 1..25: note = -
  source 1..25: mol_type = other RNA
ttcagtccttcataaactaagcttg

SEQ ID NO: 132 - RNA, 25 nt, synthetic construct
  misc_feature 1..25: note = -
  source 1..25: mol_type = other RNA
ttctgacttctgcatatactaaaga

SEQ ID NO: 133 - RNA, 25 nt, synthetic construct
  misc_feature 1..25: note = -
  source 1..25: mol_type = other RNA
tttctgacttctgcatatactaaag

SEQ ID NO: 134 - RNA, 25 nt, synthetic construct
  misc_feature 1..25: note = -
  source 1..25: mol_type = other RNA
ctttctgacttctgcatatactaaa

SEQ ID NO: 135 - RNA, 25 nt, synthetic construct
  misc_feature 1..25: note = -
  source 1..25: mol_type = other RNA
actttctgacttctgcatatactaa

SEQ ID NO: 136 - RNA, 25 nt, synthetic construct
  misc_feature 1..25: note = -
  source 1..25: mol_type = other RNA
gactttctgacttctgcatatacta

SEQ ID NO: 137 - DNA, 31 bp, Homo sapiens
  source 1..31: mol_type = genomic DNA
tttaaccagtggtccccaggctttatatttc

SEQ ID NO: 138 - DNA, 31 bp, Homo sapiens
  source 1..31: mol_type = genomic DNA
ttgtggaatgaatgaatgagcaaatgaagga

SEQ ID NO: 139 - DNA, 31 bp, Homo sapiens; identical to SEQ ID NO: 12
  source 1..31: mol_type = genomic DNA

SEQ ID NO: 140 - DNA, 31 bp, Homo sapiens
  source 1..31: mol_type = genomic DNA
gaagctggaaacgtgacaggaactgacgtgg

SEQ ID NO: 141 - DNA, 31 bp, Homo sapiens
  source 1..31: mol_type = genomic DNA
aaaatacattgtcctcatccttatgaaatta

SEQ ID NO: 142 - DNA, 31 bp, Homo sapiens; identical to SEQ ID NO: 141
  source 1..31: mol_type = genomic DNA

SEQ ID NO: 143 - DNA, 31 bp, Homo sapiens
  source 1..31: mol_type = genomic DNA
tactgcaccaggccttaggcatctttagttg

SEQ ID NO: 144 - DNA, 31 bp, Homo sapiens
  source 1..31: mol_type = genomic DNA
tgggtgtgttctaatgactagtatcaatgtg

SEQ ID NO: 145 - DNA, 31 bp, Homo sapiens
  source 1..31: mol_type = genomic DNA
ccataaaactaacaaatcacaactctgacac

SEQ ID NO: 146 - DNA, 31 bp, Homo sapiens
  source 1..31: mol_type = genomic DNA
ctatatggcatggtggcaggtccttcgtggg

SEQ ID NO: 147 - DNA, 31 bp, Homo sapiens
  source 1..31: mol_type = genomic DNA
ttgtccctgacctgtgccttgcaataggaag

SEQ ID NO: 148 - DNA, 31 bp, Homo sapiens
  source 1..31: mol_type = genomic DNA
ttttatgtatggtagccctgaaaactgctcc

SEQ ID NO: 149 - DNA, 31 bp, Homo sapiens
  source 1..31: mol_type = genomic DNA
gtggtataacccagggaaaatctctcttatt

SEQ ID NO: 150 - DNA, 31 bp, Homo sapiens
  source 1..31: mol_type = genomic DNA
aagaaatactccttaagacttgagagagcct

SEQ ID NO: 151 - DNA, 31 bp, Homo sapiens
  source 1..31: mol_type = genomic DNA
agaaagctattagatacgtatacctctatgc

SEQ ID NO: 152 - DNA, 31 bp, Homo sapiens
  source 1..31: mol_type = genomic DNA
tgccacaccttcagtctgaactggaaatgtg

SEQ ID NO: 153 - DNA, 31 bp, Homo sapiens
  source 1..31: mol_type = genomic DNA
agaaggtttccttgaggtcatgtttgaagcc

SEQ ID NO: 154 - DNA, 31 bp, Homo sapiens
  source 1..31: mol_type = genomic DNA
cacggtgcccagcctaaaacagttattatat

SEQ ID NO: 155 - DNA, 31 bp, Homo sapiens
  source 1..31: mol_type = genomic DNA
ctagatactcttcactgttgttgcacataga

SEQ ID NO: 156 - DNA, 31 bp, Homo sapiens
  source 1..31: mol_type = genomic DNA
gcatcttaaaatatttaaaatttaaaaacta

SEQ ID NO: 157 - DNA, 31 bp, Homo sapiens
  source 1..31: mol_type = genomic DNA
attgagaccatcctggctaactcggtgaaac

SEQ ID NO: 158 - DNA, 31 bp, Homo sapiens
  source 1..31: mol_type = genomic DNA
aaaatacattgtcctcatctttatgaaatta

SEQ ID NO: 159 - DNA, 31 bp, Homo sapiens; identical to SEQ ID NO: 158
  source 1..31: mol_type = genomic DNA

SEQ ID NO: 160 - DNA, 31 bp, Homo sapiens
  source 1..31: mol_type = genomic DNA
ttcgagaccagcctggccatcgtggcgaaac

SEQ ID NO: 161 - DNA, 31 bp, Homo sapiens
  source 1..31: mol_type = genomic DNA
ttccagggcttacctctagtcaacacagtag

SEQ ID NO: 162 - DNA, 31 bp, Homo sapiens
  source 1..31: mol_type = genomic DNA
aagagggcagggtgctctgagctgatttata

SEQ ID NO: 163 - DNA, 31 bp, Homo sapiens
  source 1..31: mol_type = genomic DNA
ggaggaaaatatttaagagttcattcagttt

SEQ ID NO: 164 - DNA, 31 bp, Homo sapiens
  source 1..31: mol_type = genomic DNA
tttggcttatcacagttgttcatgttatggt

SEQ ID NO: 165 - DNA, 31 bp, Homo sapiens
  source 1..31: mol_type = genomic DNA
agaccattattaaatatttgagtgctgactg

SEQ ID NO: 166 - DNA, 31 bp, Homo sapiens
  source 1..31: mol_type = genomic DNA
caccatgcctggccataaatttaattcttat

SEQ ID NO: 167 - DNA, 31 bp, Homo sapiens
  source 1..31: mol_type = genomic DNA
tcacttaatgtcattgataggttcttgcgac

SEQ ID NO: 168 - DNA, 31 bp, Homo sapiens; identical to SEQ ID NO: 167
  source 1..31: mol_type = genomic DNA

SEQ ID NO: 169 - DNA, 31 bp, Homo sapiens
  source 1..31: mol_type = genomic DNA
caagattcttcatctccagtgtgatagctgg

SEQ ID NO: 170 - DNA, 31 bp, Homo sapiens; identical to SEQ ID NO: 100
  source 1..31: mol_type = genomic DNA

SEQ ID NO: 171 - DNA, 31 bp, Homo sapiens
  source 1..31: mol_type = genomic DNA
gaccatacttaatagagtgggaattggactg

SEQ ID NO: 172 - DNA, 31 bp, Homo sapiens
  source 1..31: mol_type = genomic DNA
agtcatggcccacaggctgggcccttattac

SEQ ID NO: 173 - DNA, 31 bp, Homo sapiens; identical to SEQ ID NO: 172
  source 1..31: mol_type = genomic DNA

SEQ ID NO: 174 - DNA, 31 bp, Homo sapiens; identical to SEQ ID NO: 172
  source 1..31: mol_type = genomic DNA

SEQ ID NO: 175 - DNA, 31 bp, Homo sapiens
  source 1..31: mol_type = genomic DNA
atgaggccacttctctgttccagtcacttga

SEQ ID NO: 176 - DNA, 31 bp, Homo sapiens
  source 1..31: mol_type = genomic DNA
cccagtgctcatttctttaacacaaatattt

SEQ ID NO: 177 - DNA, 31 bp, Homo sapiens
  source 1..31: mol_type = genomic DNA
accagtagatagagatctacataaatgaaca

SEQ ID NO: 178 - DNA, 31 bp, Homo sapiens
  source 1..31: mol_type = genomic DNA
gaaagatagattaaggtccagctgagggctg

SEQ ID NO: 179 - DNA, 31 bp, Homo sapiens
  source 1..31: mol_type = genomic DNA
caccatacccagcaagaataattctgtgtat

SEQ ID NO: 180 - DNA, 31 bp, Homo sapiens
  source 1..31: mol_type = genomic DNA
aactaaattagtcccttggtattggtatcca

SEQ ID NO: 181 - DNA, 31 bp, Homo sapiens
  source 1..31: mol_type = genomic DNA
ttggagaccagcctggccaaacctgtctcta

SEQ ID NO: 182 - DNA, 31 bp, Homo sapiens
  source 1..31: mol_type = genomic DNA
gaagagatgtgaagcttccatgcccaatctg

SEQ ID NO: 183 - DNA, 31 bp, Homo sapiens
  source 1..31: mol_type = genomic DNA
acacaggggcttcattatgtagtcatgattg

SEQ ID NO: 184 - DNA, 31 bp, Homo sapiens
  source 1..31: mol_type = genomic DNA
ctctgattgattaacgtgggagcaaggaggc

SEQ ID NO: 185 - DNA, 31 bp, Homo sapiens
  source 1..31: mol_type = genomic DNA
ctctcgatgaaatcagataataaactgacaa

SEQ ID NO: 186 - DNA, 31 bp, Homo sapiens
  source 1..31: mol_type = genomic DNA
tgagttaatgttatgaatcttggaggacctg

SEQ ID NO: 187 - DNA, 31 bp, Homo sapiens; identical to SEQ ID NO: 186
  source 1..31: mol_type = genomic DNA

SEQ ID NO: 188 - DNA, 31 bp, Homo sapiens
  source 1..31: mol_type = genomic DNA
cactaatcattgatggttaattaattataca

SEQ ID NO: 189 - DNA, 31 bp, Homo sapiens
  source 1..31: mol_type = genomic DNA
taaaggaaagaggtttaatgggcccacagtt

SEQ ID NO: 190 - DNA, 31 bp, Homo sapiens; identical to SEQ ID NO: 189
  source 1..31: mol_type = genomic DNA

SEQ ID NO: 191 - DNA, 31 bp, Homo sapiens
  source 1..31: mol_type = genomic DNA
aagtagagtttcttgacagggtaccattaag

SEQ ID NO: 192 - DNA, 31 bp, Homo sapiens
  source 1..31: mol_type = genomic DNA
ttccctcaataacaactaagtaaatattacc

SEQ ID NO: 193 - DNA, 31 bp, Homo sapiens
  source 1..31: mol_type = genomic DNA
acttgcaatatgctgtggtttaggtgatgct

SEQ ID NO: 194 - DNA, 31 bp, Homo sapiens
  source 1..31: mol_type = genomic DNA
cacaggaacttattcctcactggtagaaaac

SEQ ID NO: 195 - DNA, 31 bp, Homo sapiens
  source 1..31: mol_type = genomic DNA
tttctgcagctggggggtgttcagctcctag

SEQ ID NO: 196 - DNA, 31 bp, Homo sapiens
  source 1..31: mol_type = genomic DNA
agcctgggtctgcagaacaggtttcaaggaa

SEQ ID NO: 197 - DNA, 31 bp, Homo sapiens
  source 1..31: mol_type = genomic DNA
tctgtagggcagcacccacccctgccgtcct

SEQ ID NO: 198 - DNA, 31 bp, Homo sapiens; identical to SEQ ID NO: 189
  source 1..31: mol_type = genomic DNA

SEQ ID NO: 199 - DNA, 31 bp, Homo sapiens
  source 1..31: mol_type = genomic DNA
cagcaccttcgatctgaccagtttagcaacc

SEQ ID NO: 200 - DNA, 31 bp, Homo sapiens
  source 1..31: mol_type = genomic DNA
tacagtgagttcgtaaactctgtccttccag

SEQ ID NO: 201 - DNA, 31 bp, Homo sapiens
  source 1..31: mol_type = genomic DNA
gatactgtctcctgtaattagtagaatctca

SEQ ID NO: 202 - RNA, 26 nt, synthetic construct
  misc_feature 1..26: note = SSO
  source 1..26: mol_type = other RNA
tctcgcttagcttcaacaactaagac

SEQ ID NO: 203 - RNA, 26 nt, synthetic construct
  misc_feature 1..26: note = SSO
  source 1..26: mol_type = other RNA
atctcgcttagcttcaacaactaaga

SEQ ID NO: 204 - RNA, 26 nt, synthetic construct
  misc_feature 1..26: note = SSO
  source 1..26: mol_type = other RNA
catctcgcttagcttcaacaactaag

SEQ ID NO: 205 - RNA, 26 nt, synthetic construct
  misc_feature 1..26: note = SSO
  source 1..26: mol_type = other RNA
gcatctcgcttagcttcaacaactaa

SEQ ID NO: 206 - RNA, 26 nt, synthetic construct
  misc_feature 1..26: note = SSO
  source 1..26: mol_type = other RNA
tgcatctcgcttagcttcaacaacta

SEQ ID NO: 207 - RNA, 26 nt, synthetic construct
  misc_feature 1..26: note = SSO
  source 1..26: mol_type = other RNA
ctgcatctcgcttagcttcaacaact

SEQ ID NO: 208 - RNA, 25 nt, synthetic construct
  misc_feature 1..25: note = SSO
  source 1..25: mol_type = other RNA
ttcataaagacgaggacaatgtatt

SEQ ID NO: 209 - RNA, 25 nt, synthetic construct
  misc_feature 1..25: note = SSO
  source 1..25: mol_type = other RNA
ttcataaggacgaggacaatgtatt

SEQ ID NO: 210 - RNA, 25 nt, synthetic construct
  misc_feature 1..25: note = UUCAUAAAGACGAGGACAAUGUAUU
  source 1..25: mol_type = other RNA
ttcataaagacgaggacaatgtatt

SEQ ID NO: 211 - RNA, 25 nt, synthetic construct
  misc_feature 1..25: note = SSO
  source 1..25: mol_type = other RNA
cataaggacgaggacaatgtatttt

SEQ ID NO: 212 - RNA, 25 nt, synthetic construct
  misc_feature 1..25: note = SSO
  source 1..25: mol_type = other RNA
cataaagacgaggacaatgtatttt

SEQ ID NO: 213 - RNA, 25 nt, synthetic construct
  misc_feature 1..25: note = SSO
  source 1..25: mol_type = other RNA
atttcataaggacgaggacaatgta

SEQ ID NO: 214 - RNA, 25 nt, synthetic construct
  misc_feature 1..25: note = SSO
  source 1..25: mol_type = other RNA
atttcataaagacgaggacaatgta

SEQ ID NO: 215 - RNA, 25 nt, synthetic construct
  misc_feature 1..25: note = SSO
  source 1..25: mol_type = other RNA
ttcataaggatgaggacaatgtatt

SEQ ID NO: 216 - RNA, 25 nt, synthetic construct
  misc_feature 1..25: note = SSO
  source 1..25: mol_type = other RNA
ttcataaagatgaggacaatgtatt

SEQ ID NO: 217 - DNA, 31 bp, Homo sapiens
  source 1..31: mol_type = genomic DNA
ctttctagtcagatgtctggtagtctgcagc

SEQ ID NO: 218 - DNA, 31 bp, Homo sapiens
  source 1..31: mol_type = genomic DNA
ctgcgctcagcatgatatcctgacttacagt

SEQ ID NO: 219 - DNA, 31 bp, Homo sapiens
  source 1..31: mol_type = genomic DNA
gtttctgtttctccacatccttgctaacact

SEQ ID NO: 220 - DNA, 31 bp, Homo sapiens
  source 1..31: mol_type = genomic DNA
tcatactcaagtagttgcaaaaatacttaaa

SEQ ID NO: 221 - DNA, 31 bp, Homo sapiens
  source 1..31: mol_type = genomic DNA
aacgagctttgggactatcgtaccccaagag

SEQ ID NO: 222 - DNA, 31 bp, Homo sapiens
  source 1..31: mol_type = genomic DNA
tttgtatttttagcagagatgggatttcacc

SEQ ID NO: 223 - DNA, 31 bp, Homo sapiens
  source 1..31: mol_type = genomic DNA
taaaaagttgagaaacaatagattctggtga

SEQ ID NO: 224 - DNA, 31 bp, Homo sapiens
  source 1..31: mol_type = genomic DNA
tgtgctttttcttatttatatttgcattaca

SEQ ID NO: 225 - DNA, 31 bp, Homo sapiens
  source 1..31: mol_type = genomic DNA
atgttaagatgctgggtgtatgctcatttgt

SEQ ID NO: 226 - DNA, 31 bp, Homo sapiens
  source 1..31: mol_type = genomic DNA
ttgtggttttttttagtagagatggggtttc

SEQ ID NO: 227 - DNA, 31 bp, Homo sapiens
  source 1..31: mol_type = genomic DNA
ccagcttgcaagcctgcggttcagtctagaa

SEQ ID NO: 228 - DNA, 31 bp, Homo sapiens
  source 1..31: mol_type = genomic DNA
cttgcacaggaatctattccagtatcagctt

SEQ ID NO: 229 - DNA, 31 bp, Homo sapiens
  source 1..31: mol_type = genomic DNA
tggctctagttcatctgagtccaattctgat

SEQ ID NO: 230 - DNA, 31 bp, Homo sapiens
  source 1..31: mol_type = genomic DNA
ccaaggtaaaggctgtgcctcggcccttgaa

SEQ ID NO: 231 - DNA, 31 bp, Homo sapiens
  source 1..31: mol_type = genomic DNA
atatgtatattttaaaagcagttctgttctt

SEQ ID NO: 232 - DNA, 31 bp, Homo sapiens; identical to SEQ ID NO: 231
  source 1..31: mol_type = genomic DNA

SEQ ID NO: 233 - DNA, 31 bp, Homo sapiens
  source 1..31: mol_type = genomic DNA
tttgaagcctctggtgcttatctttcttttt

SEQ ID NO: 234 - DNA, 31 bp, Homo sapiens
  source 1..31: mol_type = genomic DNA
ctccattgtttgaccctagttccagccgtga

SEQ ID NO: 235 - DNA, 31 bp, Homo sapiens
  source 1..31: mol_type = genomic DNA
cccagcggggcagtgtgtgcaaatgctgggg

SEQ ID NO: 236 - DNA, 31 bp, Homo sapiens
  source 1..31: mol_type = genomic DNA
catgcttttaacctttatagaaaacccaatc

SEQ ID NO: 237 - DNA, 31 bp, Homo sapiens
  source 1..31: mol_type = genomic DNA
caccacgcctggctaatttttgtatttttag

SEQ ID NO: 238 - DNA, 31 bp, Homo sapiens
  source 1..31: mol_type = genomic DNA
atgagcaccagcaagcaatgtttccgttatt

SEQ ID NO: 239 - DNA, 31 bp, Homo sapiens
  source 1..31: mol_type = genomic DNA
tacagaataacaaatatttcaaaatccaaaa

SEQ ID NO: 240 - DNA, 31 bp, Homo sapiens
  source 1..31: mol_type = genomic DNA
tcagcatgtggcatgtagatattaggagaag

SEQ ID NO: 241 - DNA, 31 bp, Homo sapiens
  source 1..31: mol_type = genomic DNA
aggagccctgcacgctgtgctcacgcccacc

SEQ ID NO: 242 - DNA, 31 bp, Homo sapiens
  source 1..31: mol_type = genomic DNA
taccatgtgctagtgtatgcatgtgcgtcta

SEQ ID NO: 243 - DNA, 31 bp, Homo sapiens
  source 1..31: mol_type = genomic DNA
ataattgtatttttgatggtttaaatcaagt

SEQ ID NO: 244 - DNA, 31 bp, Homo sapiens
  source 1..31: mol_type = genomic DNA
cttatcttctttaggatggtaatgatcagtt

SEQ ID NO: 245 - DNA, 31 bp, Homo sapiens
  source 1..31: mol_type = genomic DNA
tgaagtccatctgccgtttcacgtatttagt

SEQ ID NO: 246 - DNA, 31 bp, Homo sapiens
  source 1..31: mol_type = genomic DNA
tattcactgtgcagcttcagcctgcaaatgg

SEQ ID NO: 247 - DNA, 31 bp, Homo sapiens; identical to SEQ ID NO: 246
  source 1..31: mol_type = genomic DNA

SEQ ID NO: 248 - DNA, 31 bp, Homo sapiens
  source 1..31: mol_type = genomic DNA
caccatacctggctaatttttgtatttttag

SEQ ID NO: 249 - DNA, 31 bp, Homo sapiens
  source 1..31: mol_type = genomic DNA
ggctttttcattgctgctttagggactgtac

SEQ ID NO: 250 - DNA, 31 bp, Homo sapiens
  source 1..31: mol_type = genomic DNA
ggcctgaaaataattcgtggtttatggaaaa

SEQ ID NO: 251 - DNA, 31 bp, Homo sapiens
  source 1..31: mol_type = genomic DNA
tcctcacacacagttaacaggccactgatgt

SEQ ID NO: 252 - DNA, 31 bp, Homo sapiens
  source 1..31: mol_type = genomic DNA
tgtgttgcgctgtgacgtgatgacagctaca

SEQ ID NO: 253 - DNA, 31 bp, Homo sapiens
  source 1..31: mol_type = genomic DNA
atcacttgagcccaggagtacaagaccagcc

SEQ ID NO: 254 - DNA, 31 bp, Homo sapiens
  source 1..31: mol_type = genomic DNA
ctaaaggaatggaaagtgagcaaataggagc

SEQ ID NO: 255 - DNA, 31 bp, Homo sapiens
  source 1..31: mol_type = genomic DNA
tctatcatctatgcccatcctacagctacat

SEQ ID NO: 256 - DNA, 31 bp, Homo sapiens
  source 1..31: mol_type = genomic DNA
acggcgccgcacagtggaggcatctgctgca

SEQ ID NO: 257 - DNA, 31 bp, Homo sapiens
  source 1..31: mol_type = genomic DNA
ccaccgttgtgaaatccgtgccacccgagag

SEQ ID NO: 258 - DNA, 31 bp, Homo sapiens
  source 1..31: mol_type = genomic DNA
cctggcattggcgtgaggacactgtttcttc

SEQ ID NO: 259 - DNA, 31 bp, Homo sapiens; identical to SEQ ID NO: 234
  source 1..31: mol_type = genomic DNA

SEQ ID NO: 260 - DNA, 31 bp, Homo sapiens
  source 1..31: mol_type = genomic DNA
gtgaccctccggcccctctaggaccccttct

SEQ ID NO: 261 - DNA, 31 bp, Homo sapiens
  source 1..31: mol_type = genomic DNA
ccgctaccatgcctaattttttgtattttta

SEQ ID NO: 262 - DNA, 31 bp, Homo sapiens; identical to SEQ ID NO: 141
  source 1..31: mol_type = genomic DNA

SEQ ID NO: 263 - DNA, 31 bp, Homo sapiens
  source 1..31: mol_type = genomic DNA
attaaacttctttctttataaaaaaaaaaaa

SEQ ID NO: 264 - DNA, 31 bp, Homo sapiens
  source 1..31: mol_type = genomic DNA
gcagtgtcaactgcatttcaaagaacagctc

SEQ ID NO: 265 - DNA, 31 bp, Homo sapiens
  source 1..31: mol_type = genomic DNA
tctgatatgaccttagctcttttctcaaaga

SEQ ID NO: 266 - DNA, 31 bp, Homo sapiens
  source 1..31: mol_type = genomic DNA
atagttttataaagaaaaaagttggctgggt

SEQ ID NO: 267 - DNA, 31 bp, Homo sapiens
  source 1..31: mol_type = genomic DNA
tgtggcttttactctgagtaagatgggaaat

SEQ ID NO: 268 - DNA, 31 bp, Homo sapiens
  source 1..31: mol_type = genomic DNA
tggcatcaatgtcctatcctgggaagttgca

SEQ ID NO: 269 - DNA, 31 bp, Homo sapiens
  source 1..31: mol_type = genomic DNA
atctgatgcaattttcctgcttaaaaccctt

SEQ ID NO: 270 - DNA, 31 bp, Homo sapiens
  source 1..31: mol_type = genomic DNA
ccctgccagggaggggctgggtggggaggtg

SEQ ID NO: 271 - DNA, 31 bp, Homo sapiens; identical to SEQ ID NO: 270
  source 1..31: mol_type = genomic DNA

SEQ ID NO: 272 - DNA, 31 bp, Homo sapiens; identical to SEQ ID NO: 239
  source 1..31: mol_type = genomic DNA

SEQ ID NO: 273 - DNA, 31 bp, Homo sapiens
  source 1..31: mol_type = genomic DNA
caccacacctggctaatttttgtaatttttt

SEQ ID NO: 274 - DNA, 31 bp, Homo sapiens
  source 1..31: mol_type = genomic DNA
tgttatgttatatatattgttctgcaatata

SEQ ID NO: 275 - DNA, 31 bp, Homo sapiens
  source 1..31: mol_type = genomic DNA
cagtgtatttagcttcccaggtgtttgtatc

SEQ ID NO: 276 - DNA, 31 bp, Homo sapiens
  source 1..31: mol_type = genomic DNA
attagagcattcctttgagagaggtagggag

SEQ ID NO: 277 - DNA, 31 bp, Homo sapiens
  source 1..31: mol_type = genomic DNA
gtcaaaccagactgaatttatagaaattatc

SEQ ID NO: 278 - DNA, 31 bp, Homo sapiens
  source 1..31: mol_type = genomic DNA
agagccccaggttggaggcctgggccgtgac

SEQ ID NO: 279 - DNA, 31 bp, Homo sapiens
  source 1..31: mol_type = genomic DNA
cttttatgactttgctccagaagtcacatag

SEQ ID NO: 280 - DNA, 31 bp, Homo sapiens
  source 1..31: mol_type = genomic DNA
ttttttgggggtgggtaaggcacacgattca

SEQ ID NO: 281 - DNA, 31 bp, Homo sapiens
  source 1..31: mol_type = genomic DNA
gtcatattttcttgactttggcctcaggaga

SEQ ID NO: 282 - DNA, 31 bp, Homo sapiens
  source 1..31: mol_type = genomic DNA
gattctcctgcctcagcctcccgaatagctg

SEQ ID NO: 283 - DNA, 31 bp, Homo sapiens
  source 1..31: mol_type = genomic DNA
aaacaaaagcaattgtgcaaatactgtgttc

SEQ ID NO: 284 - DNA, 31 bp, Homo sapiens
  source 1..31: mol_type = genomic DNA
ctttcttatggatcctagtgtttgcatgagc

SEQ ID NO: 285 - DNA, 31 bp, Homo sapiens
  source 1..31: mol_type = genomic DNA
agttttcagctttcaacatttaatttaaggt

SEQ ID NO: 286 - DNA, 31 bp, Homo sapiens
  source 1..31: mol_type = genomic DNA
ataagtgttttacaacctactttccatgaga

SEQ ID NO: 287 - DNA, 31 bp, Homo sapiens
  source 1..31: mol_type = genomic DNA
agatcgcactactgcactccagcctgggtga

SEQ ID NO: 288 - DNA, 31 bp, Homo sapiens
  source 1..31: mol_type = genomic DNA
agatctgttgtaatccctgggaatttattac

SEQ ID NO: 289 - DNA, 31 bp, Homo sapiens
  source 1..31: mol_type = genomic DNA
tagtgaacactgcaccaataacagcatgtat

SEQ ID NO: 290 - DNA, 31 bp, Homo sapiens
  source 1..31: mol_type = genomic DNA
ttatctttgtaagctcattgctttatcttca

SEQ ID NO: 291 - DNA, 31 bp, Homo sapiens
  source 1..31: mol_type = genomic DNA
aatggtagagtgggccaggtggcttacgcct

SEQ ID NO: 292 - DNA, 31 bp, Homo sapiens
  source 1..31: mol_type = genomic DNA
atgtctatcttttaatattgtttaagataat

SEQ ID NO: 293 - DNA, 31 bp, Homo sapiens
  source 1..31: mol_type = genomic DNA
tgtcagagatggatctgggttctgtggacaa

SEQ ID NO: 294 - DNA, 31 bp, Homo sapiens
  source 1..31: mol_type = genomic DNA
tttcaaagttttattgtttgaagattcagac

SEQ ID NO: 295 - RNA, 25 nt, synthetic construct
  misc_feature 1..25: note = SSO
  source 1..25: mol_type = other RNA
gatcattaccatcctaaagaagata

SEQ ID NO: 296 - RNA, 25 nt, synthetic construct
  misc_feature 1..25: note = SSO
  source 1..25: mol_type = other RNA
cagtggcctgttaactgtgtgtgag

SEQ ID NO: 297 - RNA, 25 nt, synthetic construct
  misc_feature 1..25: note = SSO
  source 1..25: mol_type = other RNA
atgcaaacactaggatccataagaa

SEQ ID NO: 298 - RNA, 25 nt, synthetic construct
  misc_feature 1..25: note = SSO
  source 1..25: mol_type = other RNA
atgctgttattggtgcagtgttcac

SEQ ID NO: 299 - RNA, 25 nt, synthetic construct
  misc_feature 1..25: note = SSO
  source 1..25: mol_type = other RNA
ttagcaaggatgtggagaaacagaa

SEQ ID NO: 300 - RNA, 25 nt, synthetic construct
  misc_feature 1..25: note = SSO
  source 1..25: mol_type = other RNA
aattacaaaaattagccaggtgtgg

SEQ ID NO: 301 - RNA, 25 nt, synthetic construct
  misc_feature 1..25: note = SSO
  source 1..25: mol_type = other RNA
agtatttttgcaactacttgagtat

SEQ ID NO: 302 - RNA, 25 nt, synthetic construct
  misc_feature 1..25: note = SSO
  source 1..25: mol_type = other RNA
aaacagtgtcctcacgccaatgcca

SEQ ID NO: 303 - RNA, 25 nt, synthetic construct
  misc_feature 1..25: note = SSO
  source 1..25: mol_type = other RNA
agcatttgcacacactgccccgctg

SEQ ID NO: 304 - RNA, 25 nt, synthetic construct
  misc_feature 1..25: note = SSO
  source 1..25: mol_type = other RNA
agctgtaggatgggcatagatgata

SEQ ID NO: 305 - RNA, 25 nt, synthetic construct
  misc_feature 1..25: note = SSO
  source 1..25: mol_type = other RNA
aatacaaaaaattaggcatggtagc

SEQ ID NO: 306 - RNA, 25 nt, synthetic construct
  misc_feature 1..25: note = SSO
  source 1..25: mol_type = other RNA
tccccacccagcccctccctggcag

SEQ ID NO: 307 - RNA, 25 nt, synthetic construct
  misc_feature 1..25: note = SSO
  source 1..25: mol_type = other RNA
gataaagcaatgagcttacaaagat

SEQ ID NO: 308 - RNA, 25 nt, synthetic construct
  misc_feature 1..25: note = SSO
  source 1..25: mol_type = other RNA
agccaacttttttctttataaaact

SEQ ID NO: 309 - RNA, 25 nt, synthetic construct
  misc_feature 1..25: note = SSO
  source 1..25: mol_type = other RNA
aaatcccatctctgctaaaaataca

SEQ ID NO: 310 - RNA, 25 nt, synthetic construct
  misc_feature 1..25: note = SSO
  source 1..25: mol_type = other RNA
ccataaaccacgaattattttcagg

SEQ ID NO: 311 - RNA, 25 nt, synthetic construct
  misc_feature 1..25: note = SSO
  source 1..25: mol_type = other RNA
tcttaaacaatattaaaagatagac

SEQ ID NO: 312 - RNA, 25 nt, synthetic construct
  misc_feature 1..25: note = SSO
  source 1..25: mol_type = other RNA
caaacacctgggaagctaaatacac

SEQ ID NO: 313 - RNA, 25 nt, synthetic construct
  misc_feature 1..25: note = SSO
  source 1..25: mol_type = other RNA
taaattcccagggattacaacagat

SEQ ID NO: 314 - RNA, 25 nt, synthetic construct
  misc_feature 1..25: note = SSO
  source 1..25: mol_type = other RNA
gggttttctataaaggttaaaagca

SEQ ID NO: 315 - RNA, 25 nt, synthetic construct
  misc_feature 1..25: note = SSO
  source 1..25: mol_type = other RNA
ctatttgctcactttccattccttt

SEQ ID NO: 316 - RNA, 25 nt, synthetic construct
  misc_feature 1..25: note = SSO
  source 1..25: mol_type = other RNA
cggcccaggcctccaacctggggct

SEQ ID NO: 317 - RNA, 25 nt, synthetic construct
  misc_feature 1..25: note = SSO
  source 1..25: mol_type = other RNA
ggattttgaaatatttgttattctg

SEQ ID NO: 318 - RNA, 25 nt, synthetic construct
  misc_feature 1..25: note = SSO
  source 1..25: mol_type = other RNA
gaattggactcagatgaactagagc

SEQ ID NO: 319 - RNA, 25 nt, synthetic construct
  misc_feature 1..25: note = SSO
  source 1..25: mol_type = other RNA
taaattaaatgttgaaagctgaaaa

SEQ ID NO: 320 - RNA, 25 nt, synthetic construct
  misc_feature 1..25: note = SSO
  source 1..25: mol_type = other RNA
acagtatttgcacaattgcttttgt

SEQ ID NO: 321 - RNA, 25 nt, synthetic construct
  misc_feature 1..25: note = SSO
  source 1..25: mol_type = other RNA
ctacctctctcaaaggaatgctcta

SEQ ID NO: 322 - RNA, 25 nt, synthetic construct
  misc_feature 1..25: note = SSO
  source 1..25: mol_type = other RNA
gcagatgcctccactgtgcggcgcc

SEQ ID NO: 323 - RNA, 25 nt, synthetic construct
  misc_feature 1..25: note = SSO
  source 1..25: mol_type = other RNA
cgggtggcacggatttcacaacggt

SEQ ID NO: 324 - RNA, 25 nt, synthetic construct
  misc_feature 1..25: note = SSO
  source 1..25: mol_type = other RNA
tggggtacgatagtcccaaagctcg

SEQ ID NO: 325 - RNA, 25 nt, synthetic construct
  misc_feature 1..25: note = SSO
  source 1..25: mol_type = other RNA
agggccgaggcacagcctttacctt

SEQ ID NO: 326 - RNA, 25 nt, synthetic construct
  misc_feature 1..25: note = SSO
  source 1..25: mol_type = other RNA
ttgcaggctgaagctgcacagtgaa

SEQ ID NO: 327 - RNA, 25 nt, synthetic construct
  misc_feature 1..25: note = SSO
  source 1..25: mol_type = other RNA
gctgtcatcacgtcacagcgcaaca

SEQ ID NO: 328 - RNA, 25 nt, synthetic construct
  misc_feature 1..25: note = SSO
  source 1..25: mol_type = other RNA
gaatcttcaaacaataaaactttga

SEQ ID NO: 329 - RNA, 25 nt, synthetic construct
  misc_feature 1..25: note = SSO
  source 1..25: mol_type = other RNA
gatttaaaccatcaaaaatacaatt

SEQ ID NO: 330 - RNA, 25 nt, synthetic construct
  misc_feature 1..25: note = SSO
  source 1..25: mol_type = other RNA
atttctataaattcagtctggtttg

SEQ ID NO: 331 - RNA, 25 nt, synthetic construct
  misc_feature 1..25: note = SSO
  source 1..25: mol_type = other RNA
agactgaaccgcaggcttgcaagct

SEQ ID NO: 332 - RNA, 25 nt, synthetic construct
  misc_feature 1..25: note = SSO
  source 1..25: mol_type = other RNA
taagtcaggatatcatgctgagcgc

SEQ ID NO: 333 - RNA, 25 nt, synthetic construct
  misc_feature 1..25: note = SSO
  source 1..25: mol_type = other RNA
cagactaccagacatctgactagaa

SEQ ID NO: 334 - RNA, 25 nt, synthetic construct
  misc_feature 1..25: note = SSO
  source 1..25: mol_type = other RNA
ttcataaggatgaggacaatgtatt

SEQ ID NO: 335 - RNA, 25 nt, synthetic construct
  misc_feature 1..25: note = SSO
  source 1..25: mol_type = other RNA
gttttaagcaggaaaattgcatcag

SEQ ID NO: 336 - RNA, 25 nt, synthetic construct
  misc_feature 1..25: note = SSO
  source 1..25: mol_type = other RNA
atggaaagtaggttgtaaaacactt

SEQ ID NO: 337 - RNA, 25 nt, synthetic construct
  misc_feature 1..25: note = SSO
  source 1..25: mol_type = other RNA
acttcccaggataggacattgatgc

SEQ ID NO: 338 - RNA, 25 nt, synthetic construct
  misc_feature 1..25: note = SSO
  source 1..25: mol_type = other RNA
acagaactgcttttaaaatatacat

SEQ ID NO: 339 - RNA, 25 nt, synthetic construct
  misc_feature 1..25: note = SSO
  source 1..25: mol_type = other RNA
ctgaggccaaagtcaagaaaatatg

SEQ ID NO: 340 - RNA, 25 nt, synthetic construct
  misc_feature 1..25: note = SSO
  source 1..25: mol_type = other RNA
cgcacatgcatacactagcacatgg

SEQ ID NO: 341 - RNA, 25 nt, synthetic construct
  misc_feature 1..25: note = SSO
  source 1..25: mol_type = other RNA
cccatcttactcagagtaaaagcca

SEQ ID NO: 342 - RNA, 25 nt, synthetic construct
  misc_feature 1..25: note = SSO
  source 1..25: mol_type = other RNA
tattcgggaggctgaggcaggagaa

SEQ ID NO: 343 - RNA, 25 nt, synthetic construct
  misc_feature 1..25: note = SSO
  source 1..25: mol_type = other RNA
ttgcagaacaatatatataacataa

SEQ ID NO: 344 - RNA, 25 nt, synthetic construct
  misc_feature 1..25: note = SSO
  source 1..25: mol_type = other RNA
gtgacttctggagcaaagtcataaa

SEQ ID NO: 345 - RNA, 25 nt, synthetic construct
  misc_feature 1..25: note = SSO
  source 1..25: mol_type = other RNA
atgcaaatataaataagaaaaagca

SEQ ID NO: 346 - RNA, 25 nt, synthetic construct
  misc_feature 1..25: note = SSO
  source 1..25: mol_type = other RNA
tgatactggaatagattcctgtgca

SEQ ID NO: 347 - RNA, 25 nt, synthetic construct
  misc_feature 1..25: note = SSO
  source 1..25: mol_type = other RNA
aatacgtgaaacggcagatggactt

SEQ ID NO: 348 - RNA, 25 nt, synthetic construct
  misc_feature 1..25: note = SSO
  source 1..25: mol_type = other RNA
tgagaaaagagctaaggtcatatca

SEQ ID NO: 349 - RNA, 25 nt, synthetic construct
  misc_feature 1..25: note = SSO
  source 1..25: mol_type = other RNA
ccaggctggagtgcagtagtgcgat

SEQ ID NO: 350 - RNA, 25 nt, synthetic construct
  misc_feature 1..25: note = SSO
  source 1..25: mol_type = other RNA
tcgtgtgccttacccacccccaaaa

SEQ ID NO: 351 - RNA, 25 nt, synthetic construct
  misc_feature 1..25: note = SSO
  source 1..25: mol_type = other RNA
ggcgtgagcacagcgtgcagggctc

SEQ ID NO: 352 - RNA, 25 nt, synthetic construct
  misc_feature 1..25: note = SSO
  source 1..25: mol_type = other RNA
aaatacaaaaattagccaggcgtgg

SEQ ID NO: 353 - RNA, 25 nt, synthetic construct
  misc_feature 1..25: note = SSO
  source 1..25: mol_type = other RNA
acggaaacattgcttgctggtgctc

SEQ ID NO: 354 - RNA, 25 nt, synthetic construct
  misc_feature 1..25: note = SSO
  source 1..25: mol_type = other RNA
ggggtcctagaggggccggagggtc

SEQ ID NO: 355 - RNA, 25 nt, synthetic construct
  misc_feature 1..25: note = SSO
  source 1..25: mol_type = other RNA
agtccctaaagcagcaatgaaaaag

SEQ ID NO: 356 - RNA, 25 nt, synthetic construct
  misc_feature 1..25: note = SSO
  source 1..25: mol_type = other RNA
atgagcatacacccagcatcttaac

SEQ ID NO: 357 - RNA, 25 nt, synthetic construct
  misc_feature 1..25: note = SSO
  source 1..25: mol_type = other RNA
cagaatctattgtttctcaactttt

SEQ ID NO: 358 - RNA, 25 nt, synthetic construct
  misc_feature 1..25: note = SSO
  source 1..25: mol_type = other RNA
aaatacaaaaattagccaggtatgg

SEQ ID NO: 359 - RNA, 25 nt, synthetic construct
  misc_feature 1..25: note = SSO
  source 1..25: mol_type = other RNA
agaaagataagcaccagaggcttca

SEQ ID NO: 360 - RNA, 25 nt, synthetic construct
  misc_feature 1..25: note = SSO
  source 1..25: mol_type = other RNA
ggctggaactagggtcaaacaatgg

SEQ ID NO: 361 - RNA, 25 nt, synthetic construct
  misc_feature 1..25: note = SSO
  source 1..25: mol_type = other RNA
ttttttttataaagaaagaagttta

SEQ ID NO: 362 - RNA, 25 nt, synthetic construct
  misc_feature 1..25: note = SSO
  source 1..25: mol_type = other RNA
ggtcttgtactcctgggctcaagtg

SEQ ID NO: 363 - RNA, 25 nt, synthetic construct
  misc_feature 1..25: note = SSO
  source 1..25: mol_type = other RNA
ccacagaacccagatccatctctga

SEQ ID NO: 364 - RNA, 25 nt, synthetic construct
  misc_feature 1..25: note = SSO
  source 1..25: mol_type = other RNA
tcctaatatctacatgccacatgct

SEQ ID NO: 365 - RNA, 25 nt, synthetic construct
  misc_feature 1..25: note = SSO
  source 1..25: mol_type = other RNA
ccccatctctactaaaaaaaaccac

SEQ ID NO: 366 - RNA, 25 nt, synthetic construct
  misc_feature 1..25: note = SSO
  source 1..25: mol_type = other RNA
gtaagccacctggcccactctacca

SEQ ID NO: 367 - RNA, 25 nt, synthetic construct
  misc_feature 1..25: note = SSO
  source 1..25: mol_type = other RNA
tgttctttgaaatgcagttgacact

SEQ ID NO: 368-369 - skipped (2 entries)